SEQUENCE LISTING (87 sequences)

SEQ ID NO: 1 - DNA, 1141 bp, Mus musculus
aaatatgagactggggatgtctgtagctcattctgctctggagcccaccaagaacgatagtcaattccagaaaccgctatgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctagtgcgttatgcctaagcatatcagtttgtggacattcctcactgtggtcagaaaatatatcctgttgtcaggtatctgacttatgttgttctctacgaagaactgacaatatgaatgttgggacactattttaattatttttaatttattgataatttaaataagtaaactttaagttaatttatgattgatatttattatttttatgaagtgtcacttgaaatgttatatgttatagttttgaaatgataacctaaaaatctatttgatataaatattctgttacctagccagatggtttcttggaatgtataagtttacctcaatgaattgctaatttaaatatgtttttaaagaaatctttgtgatgtatttttataatgtttagactgtcttcaaacaaataaattatattatatttaaaaacc

SEQ ID NO: 2 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 3 - DNA, 1083 bp, Mus musculus
aaatatgagactggggatgtctgtagctcattctgctctggagcccaccaagaacgatagtcaattccagaaaccgctatgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagagataagctggagtcacagaaggagtggctaaggaccaagaccatccaattcatcttgaaatcacttgaagaatttctaaaagtcactttgagatctactcggcaaacctagtgcgttatgcctaagcatatcagtttgtggacattcctcactgtggtcagaaaatatatcctgttgtcaggtatctgacttatgttgttctctacgaagaactgacaatatgaatgttgggacactattttaattatttttaatttattgataatttaaataagtaaactttaagttaatttatgattgatatttattatttttatgaagtgtcacttgaaatgttatatgttatagttttgaaatgataacctaaaaatctatttgatataaatattctgttacctagccagatggtttcttggaatgtataagtttacctcaatgaattgctaatttaaatatgtttttaaagaaatctttgtgatgtatttttataatgtttagactgtcttcaaacaaataaattatattatatttaaaaacc

SEQ ID NO: 4 - protein, 165 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEISWSHRRSG

SEQ ID NO: 5 - DNA, 1197 bp, Homo sapiens
gtctcaatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaa

SEQ ID NO: 6 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 7 - DNA, 1006 bp, Homo sapiens
gtctcaatattagagtctcaacccccaataaatataggactggagatgtctgaggctcattctgccctcgagcccaccgggaacgaaagagaagctctatctcccctccaggagcccagctatgaactccttctccacaaacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaa

SEQ ID NO: 8 - protein, 136 aa, Homo sapiens
MCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 9 - DNA, 5483 bp, Artificial Sequence
  5′ homologous arm
aggtcttctcgtgacttcagaaagcatatcaaactatcacaactggtacatataactgtatcttttaaaggattttaaatcttagtatttgctgaaatacttagtgtttactatttcagcatctcatctgagttccgagaagttgtggattctctccttatccacatcagccccgcctcccccacggtcagctccttgctgcctgcttgtgatagttcttactatcaagatacgcagtttctcttgcacaagcaagactatcatactaggtaacttagtcccctgggaagatacaaccagatcttaagtaaaaagagagaaacacttcaatggtaatatccttttatcaataatccatttgtcaccttgtgaaacaggccccagtaatgaatttcaagatgaagtaatgtgacatatgaaaagaagagtgataaggtcagcctttgtccatgaaaatgatctatacatcttgccatccctgaataccaattagctactaatgcccactgcgagtccaatcaggactagtctgtgttccagaccagctgaaaagcaaatatcaagtcaagctgcaaaatgtgcatggtgtcaaacaaccagcataacttaataatttagagatgccattactgcatgaacaaacaataagttaacctttgggaaatcagtgattattttcaatgtgtattatcaatactaagctatgaatacattccctgtgctttgtgaacagtgagcatctatatacttcacttgatgcttcccttctgaactccaactagtttatattcagagagttactctgtgttgtatatgcagagaaatacaagaaagaaagaaaagccaagccaggtctggtggcatgaatttgtaattctaacttttcagaaagctgagaatcactactaacaatctgaaagaacaagaaccaccaacaataattactaagaaatgtgctattttattgcactggcatgttttcacacagactctaacttcttgaatttgtttattttgcctgaggccaccaagatcctggacatgtaaaaatggaaagcatcctaaacagctgaaattagtttactgaattttctttgggtatgagcaagatgatggcatcagactttcaagataacattcaagagttatttctcaattctgaggacatatcaggcaacagcccatgatgagtagattttggggtgttgagtggagagctgtagaatcacacagtgtggacagactgttggagcagggaaagaaattggaaagtgtaatatgtggagggcttaggtaggaaaacactgtgctctgacaccctcctgttagtcatcatctcaatgtttgaatctgaaaacagttgtactactaaaatacaacccacggatttagtaatgtagaaagaaaagcgaacaaacaaggacaatttagggaaactctccattcagccaaaagatttgcaacctcattctgtctgaaatctgcattccattcagtaaggctattcagatcttcacaaatctagacaaaagaacagctacataatacctgggcctgagtctccttctaaatacaacttgcccaagactacatacctgggtcccagagtagcttccctagagctgcaatgaataaactagtcatagaggaatatttatcctgtatttacagagccatgagagaatctgatgcagccagcagctgttgcttaggcaccaaacctccaaacagatcttttgccatttccaggcactgtgatcccttgtaaagctgactagtgggaaagtcaggcctgctctctggtcactcactaccttgagttcctgaattttacctacatggaagaaatctggtttcctggatacggaatctcaataagtcatcccacaagagcccagaccttcaccaatatggaggagtcacaggccctgtcagaatagtgacagcagacagatagccctggctctttgaatacttcaaggttatccagcacagaagggacagcattgtagagatgtgacaatagtaggtctgtgggtcaaggaatctctctctcccctgctgtaccaccctaccccaccacatatcacatatacacagagcaagtcacacacacacacacatacacgggagggatgggagagaaagagagagagagagagagagagagagagagagagagagagagagagagagagagagaggagagagagagagagagagaatattcagcaatttcaatgtatcacctgtaaattttttttttggccttaacttgcttaccaggatctgaattcttctgtaacactttaaatcaatccaggtagatctgcgatgaagcaactagacctagtgtctcagatgtggagagtgtttcacacatccaaagttttttgtggaaatggttcaatatgaagattaggcctttcctaccttcatgaatccagcttgcaagactacagcagagactttattcagaggaactaagacattagcaaataactgataaaaaggaagggaggtaaaatacatcacagtcaggccaaaagaatcccctggttttgacacaacatgtgatgctttcaactagttcctactaccttaattttaagggaaaattgatactttgcatgcttagtttctgctcatggctctgaaccgagagaactgagatctttggagaagccaaagtggtttaagtgacttaccagaataaatgagctggtttctcttagctgaggtttcctaatggtgaagcacaagtagcagccactgaaaatcaaccccacagagagaggcaagcacagaaagtcagcccaagggatcttcccggaccggggagagttcaggatatcaactcgatgtcttatcaccaaccagctcggagaggccatcttatcgacacttagggatcagagcactctcattaagataagacagaaatgtgtagcaggtccttaggcagggagactgttccttgctatccatagagccactcctaaaatgcctgggagtttctagttttctagggataagatatctccatgcatagcaaaataacaagagcaaagaagagatgaaatcatagggcagatttgaattaaacttagcatagaagatctaggtttagccctgttgcaggttggaaaaatctaaaactgggtcaagaagttgttaggtactacactgagcaacactgaggctgtaaggccagaactgaaactgaactgtctggcagctatcaagaggttagggcaattagcctggctctcctttaaaattagccagtgccttccaaataaacacagcacccttctccctaacagtcgctctccaaataacaacaatacgttttctcactacaagacattttctagagcctctctcccagaagaagctgttaaattaaccattgtttcactttgcaaacatcttctgccttggtgacaaatgaaactacgttctaaaatcaactgcaagttctctagtaccagtaacttctcttttctctttgagatacgctttttagttaaattgcagacctagtcatctccaaccccagccaaagagcacatcacccaagcctgagagtgtggaaagcatttctcttctagagaggtaaatgtccttcttccacttacctgaaatgtactaataaactctggaatccatcagcacatagaaaagtgcatgtgagtagtaattcagagggatacaatcagccccatacaaaagaagagaactgtgatacacacattctatccctgatctcttgatttgcatctagcttagcatcaggagatctaggtgtagccctgtttcaggttggaaaatctaggctgggtcgagaagttgtgaatgcaacttcttgcaatgctgaagtcctcaaatttttagttagtaatgacaatgtatactagcctttcttgtttattaaaacatactaaaatatgaatgagatttggggttaagtaagtgcagctaaggaaatgtgtgcctcaagttttgctgtgatcttaaaactgcagaaaaaagtgtacgtacacacacacacacacacacacacacacacacacacgaggagtttttcaaatgggaaacccatcaagatacagagaaaagaatctgtgatggtagaaacagagtttagagttactgtgctaatattttggtttaggaagagtctttttctcccataatcaaatgccaatcaaaggcagtggtactaacaaagatctatgctggtggcatacaaagggacaacacagaatcaccctaccttccaacccttgaatatctgactagtagaagctcaagctctggggttgtcaaggccatttatgcctgagaatcatactcagaaacttgaaggatagcgttatgttgtaggctgtaacttctcacataaaaacacttaacagaaggcttcggggagagtctaggagtaaagcacagctgggtaattactaagtaagggacgtagtttgagcatggttttttttctgcacgtgtgaaggtgcagtgtcattctgtatattcagtgccaactgaggctataagagggcatcagaaccccaggaacaagagtcacagaaggtttaaaaccaccatgtagaaactgaacccacaacctcttcaaagagcagtaaatgcaatcggaagttatagagttggagcttatactttcttagtcatcttcatggcagctccgtatgatgatctattattattctcatgttaaagagtaaataaacaagaacacagatcttgagctaacttagccattgtcaaacagctagcaatggagttgggcttctaattcaaattgttactcaaaggctatggtctccacgtgtacccttggaaagctagatagacagcatccagaccattaacatacagtgtgtacctctatgtataaatatgtatttttacacacatttatgtatgaaatgtatatgtataaatatatgagctgagaaagcctcttccagatgaggctgacaagagttccactaaaactaccggggtttaggcatctcctgtgggaccattcttctgatgtcttgtttaaacatttttatcatggatgtatgctcccgacttaaaaagcaccttttttaaaaaactaaaaacagaaatctgaatgttgtagtaagtgtaacaatcttaagtttattcagtaatttaaaaaaattgttaagcggagaaaagaaactctgtactaacagaggcctgagaaagcacacggcagggaataggggaaatggcttccttcattgctggacacagactgagctccaggctgtttcagctgcctttttaaggctcaagggcactaaaagtaaaaccatcctgcttcctctccccattttcattttcacctaaaatcccctagtccctttgtgaagaccagggcttcacacggtgaaagaatggtggactcacttctttcaataggctgacctagtatgtacactaag

SEQ ID NO: 10 - DNA, 4770 bp, Artificial Sequence
  3′ homologous arm
cttacccaacatgagcaaggtcctaagttacatccaaacatcctcccccaaatcaataattaagcactttttatgacatgtaaagttaaataagaagtgaaagctgcagatggtgagtgagagatgccatgagaaagcattgcatataccacattagttaatttcaggtcttgtacattcttttctggacatgagagagtaagggatctaactaagccaccttttggaaacataaaacataatctctgatttgaattcaagtctacctccctctaggtccatttttaacttttagttgtaatttgaagacagatatagaaaaatctcaaaacattttaatatgaattatacacttagagttgatgtcacagattctgagaccatgggactacttagataagatatagctccaaaagataaaagcgccaaaataatatccagaagttctgcctccctcgtctggagtctccatgcactgcatacctcctattagtgtctgccattatatatcataccttaaaactgaaggagctttctatccaactagcatatgggtccctcaagaaagcagactctagtgttttaaccttttcgtgctatatataggtaaggagcctgaacaaaggagacccctataagtatttgctgaatgaaaagagaatagttaatcacagtataacaaaagtcagttcttggtaaatacagagcatttgggtgacattacagtgatgtgttattgtcttttaaaaaaagtagaaaagaatggaaatgaaacattttaaggatttctaaataaggggcagatacaagagtattttgggttttagcccagactatactgtagggggaaagcctgtctcaactttatcccaatttcatatatgtatgtccatatatgtgcatgtatgtgcacatgcacacatgggtgtgtatgtgtgtgtgtatggtgattatgatgatgatgaagagatgatgtctataacttaatgtggttcttcctatttctgtacaaaactgagaatttggtgccaattctctctctctctctctctctctctctctctctctctctctctctctctctctctctctctctctctttttcttcttcttcttcttcttcttcttcttcttcttcttcttcttcttcttcttcttcttcttcttcttcttcttcttcttcttcttcttcttcttcttctttttattattattttatgtacattccagccattgcccccaggatcccctcccacagttcctcatcccattcctctttccccttgcctccgagagggtgctcccccctaccagacctccctcttccctggggtctcaagtctcttgaggattaggtgcaccttctcccactgaagccagaccagacagtcctctgctacatatgtgcctgtggccctcagaccagcccgtgtatgctgcctcattggtggctcagtctctgggagttccctggggtccaggttagttgagacttctgatcttcctatggggtagcccttcccttcagcttcttcaatccttcccataattcaaccataggggtttccaacttcagtccaatgattgggtttaagtgtctatttatgtctcagtcagcttcggattgggtctgaggacagccattttacactcttgtctgtaagcacatcatagcatcagtaatagtgtcaggccttgaacaccctccagccccctgtcatgagatggatcccaatatgggctagtcattggaccaactttccttcagtttcttctccatttttgtccctgcagttcttttagacaggaacaattttgggtcagaaattttgactatgggttactaatccagtccctctacttgtggtcctgtctatctactggaggtggactctctgagttccctctccccattgttgagcatattggctaaggtcaccccccccccattgagtcctgagagtctctcacctcctgggtctctggtactttctagagggttcccctacccctcaccatccaaggctgcatatttccattcattctcctggccctttggtcttctctcctgtccaaccccaatctaatcttgttccctttcccctttcactcccctctctcacccaggtcccttcttccctctgcctctcatgattattttattccatatctaagtgggattaaagcatccccacttgggtctttctgcttgttacacttcttatggtctgtgggttgtatcctacatattctgtacttcttggctaatatctatttatcagtgagtatataccatgcatgtccttttgggtctgggttacctcacttaggatgatattttctacttccatccatttgcctgtaaaatttgtgatatcctcatttttaatagcttaataatattccattgtgtaaatgaaccacattttctgtaactattctttggttgagggactgccgtggactggacttagtcggtccctcaacccacaagaaaccagagtttcagtactcatgtgggcaaggagatggcaaaaaaatgacagacactgacacacagagagtgctgtatctgaatataatttctcaaagcgagcatcagacttatattacagaagaaaacaaataagttatgtgacacataagccaaggtacattgaagttatctgacacaaaacagaaaaattcataaagactgacaggaaccaggcagtggttgcaactgagataaaaggcagccctttctaaagtcagccattaggaagccaggtgaggatttcacaccctagttacaatttatgctattccactgagccttgtgaaagcttgtaccaagggggttcagctcttgcttatgaataatgcaatactgtagttccaccttaaaccacatccctccttcttcctaggccattgtaaattcctgcatatgagagtgaccggctgtaattctaagcttactttgtagaacttgccctgagatttttagctcttatccagtaaaatactgcaagaaagcatgcaaaaccctccacactaactcagggacaaatctggttatggggggggctggtgagatggctcagtgggtaagagcacccgactgctcttccgaaggtccgaagttcaaatcccagcaaccacatggtggctcacaaccacccgtaatgagatctgatgccctcttctggtgcgtctgaagacagctacagtgtacttacatataataaataaataaatctttaaaaaaaaaaaaaaaaaaaagatttaaaaaaaaaaaaataaaaataaaaaaaaaaaatctggttatggggcaccagagactctccaggagacaagtttctgtgaaactttttgcctcaggactgtgtccaagcttttgggcttgccacgcagacttcactggagtgggtgtggcaagagacatctgagttgtttccagatttgtttctggctattacaaataaggctgctatgagaatagtggagtacgtgtccttgtgatatggtggggcatcttttgggtatatgcccaagagtggtatagctgggtctcctagtagtactatgtccagttctctgaggaacctccagattgatttccagagtggttgtaccagtttgcaatcccaccagcaatggaggagtgttcctctttctccacatcttcaccagcatctgctgtctcctgagttttttatcttagccattctgattgatgaaaggtgaaatctcagggtagttttgatttgcatttccctgatgaataaggatgttgaacatttctttaagtgcttcttggcttctgatatccttctgttatgaattctctgtttaactctgtaccccatgttttaaagaattttttattatatatatatatatttgagacagggtttctctgtatagccctggctgtcctggaactcactttgtagaccaggctggcctcaaatctgcctgcctctgcctcccaagtgctgggattaaaggcgtgtgccaccacgcactgcttattatatattttttacagtccagtcactgccccctttccagtctgctctcccacagttcttcatcccattcctcttcccccctgtcttcaaggggatgttctctagaacccctgctaggcctccccactcccagaggcctcaagtctctaaggttgggtacctgttctcccactgaagccagaccaggcagttgtatcccatttttaaattgggttatttgttttgttttttggaggttaacttcttgagctctttatatattttagatattagcactctttcggatgtaaggttagtaaagttttttcccccaatctgtaggttgccaatttatcctattgatggtgtcttttgttttacagaagctttttggtttcatgaggtcctatttgtcagttgttgatcttagagcctgagccattggtgttccttacgatcaagaaaatttcccctgtgccaatgagttcaaagctccttcccactttctcttctattagattcaatgtatctggttttatgttgaggtccttgatccacttagacttgaactttgtgcaaagtgataaatatgggtctattttcattgttctgcatacatacatacatacatacatacatacatacataaatacatacatagttataccagcaccatttattgaagatgttttcttttttccactgcatgattttggcttctttgtcaaagattaagtgtacataggtgtgtgggtttatgtctgggtctttgatt

SEQ ID NO: 11 - DNA, 12726 bp, Artificial Sequence
  fragment of human DNA
actagcttcagagaagtttgcaatcagggcactctcttccaagcctagagacccagggaaaggggtacgggggtgtcccaaggcaaagagaatctacactttttgcccccggagaggctacttccctcccaagatgcctgggattttccacttcagcagggggaaggtaagtcacatagcaaaataatgagggcacagaacagatgacctccctatagagttttgaatgagaaacacagcagggcagatgtgccccttctctagtctaggaggagctaggtccagcccctgaacatcctccccctcagaaaagctgaggccagactaagaattcaccagaccaaggagctacaacaggacatcagagctgaggctgcaaagccaggactgagaccagaccaggcaggaaactgtcaagagctttggtcaccaggcctggctgccctccaacatcagctggctctttctaaattgacacaccacatgtccctaaaattctctcttcaagtaataccaccatcaaagcaggacatttcccagagccttagagcctggtgtctgctcagtgggactcaaccccagaagaagctgttaaatcacccactgtttcagtttacaaacttcttacgacttggcaacaagtgaaactacattctggcagcaactgcaagttccctagtacccaggacttcccgttttttcttgctgtactccctcctgttaaatcacagactcatccatctccaacccccagaatatagagaaagagcacaacactacatcttaactcctgagacgtggagaacacttctcctcctgagagcttaagtaccaaatggaagctacttttcccccttggtctcaaatgtattactagattctgaactggactccaccatcacgtaagaaagcagtcatgggcagtaattctgggagatccagataggacatgccagccccacactggtggcataggaagccaagttgctgcttcctccctgtgcactcccatttgtctggcctctcttgatctcagctggcgctcacttcacatcagctatgatgcaatccagcaactaaagtattagttaataaatgctgacagcacagccttttctggtcacgtattcatactaaaatacgggggagagttggggggagagggggatatatgggaaatctctgtaccttcctctccattttgctatgacctaaagctgccctttaaaaaatacaaggggctgggcacagtggttcacgcctgtaaacccagcactttgggaggccgaggcgcgtggatcacctgaggtcaggagttcaagacccgcctggccaacatggcaaaaccccgtttctactaaaaatacaaaaagtagctgggcgtggtcgcatgcatctgtagtcccagctactcaggaggctgaggcaagagaattgcttgaacctgggaggcggcggttgaagtgagccaagatcatgccattgccctccagcctgggcaacagagcaagactccttctcaagagaaaaaacaaaacaaaacaagaaaaaacaaagaatgagctctccacgcgaaaaatccattgagatgcaaaggaaggaagctatcattgtggaattgcacatgtcagttacattaacgtttttggagcaaggtagagctcatctctcccacaagcaaattccagcccaaagcattgatactaataaagtgccatgctgcgatgtgcagggggcagacagtgtctccaagctccctacacacatgccttcccacagtttgccctttcttgaccccagaagcatcaggccccttcaccctcgagggccactatcaggagtttgaattaatggcaatcaccatgcacagggaaggctgtggaattctgacataaaaacacttagtggagggcttggaaaaagtctagtaggagcaagacgcaagctggactaattatctaaaacaagagacctggtttggggatcttaatgttctcaaaaaagaaaattattattatttttcattttgcactttgtgccataaaacattttcaacaaaacatagaatctcatttcttttgagggaaaatgattgggagaccagctcattgctggcacagaggcctggttcattcataattccttcataggcaagacaccaggtgaaccgatatagccgagctggaagagctctccaaggcagagactctgagccaaggaatgttcaaagagctagcatgtattgtgggattactatgcgccaggaattttttacactgcatcacgttccatcttcacaacagccctagaaaggaagaactattattacccccgttttataggtgaataaacaagggcacaggtccttgatgtaacagccaggatcaaacagctgggaagacgagaaaacctttcccaggctaggataacagaggatttggttgaaaatacaggcaattaggtgctacctctgggaaaaggggccaggagaggaaggagacacttttccctgcatgccctgatgtcctatttgaacattttatcatgaacacgaacttcctatttaaaaaacactttttattgaaaagataaatctgtgtgttgtattgtgtcactcagttcaagtacttgaaatttattgaattgtattttctaaaaaatagatagttgagtaaaagcaagctcacattacatagacggatcacagtgcacggctgcggagctgggagcagtggcttcgtttcatgcaggaaagagaacttggttcaggagtgtctacgttgcttaagacaggagagcactaaaaatgaaaccatccagccatcctcccccattttcattttcacaccaaagaatcccaccgcggcagaggaccaccgtctctgtttagacaatcggtgaagaatggatgacctcactttccccaacaggcgggtcctgaaatgttatgcacgaaacaaaacttgagtaaatgcccaacagaggtcactgttttatcgatcttgaagagatctcttcttagcaaagcaaagaaaccgattgtgaaggtaacaccatgtttggtaaataagtgttttggtgttgtgcaagggtctggtttcagcctgaagccatctcagagctgtctgggtctctggagactggagggacaacctagtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggtgagtagtattctccccctttctgccctgaaccaagtgggcttcagtaatttcagggctccaggagacctggggcccatgcaggtgccccagtgaaacagtggtgaagagactcagtggcaatggggagagcactggcagcacaaggcaaacctctggcacagagagcaaagtcctcactgggaggattcccaaggggtcacttgggagagggcagggcagcagccaacctcctctaagtgggctgaagcaggtgaagaaagtggcagaagccacgcggtggcaaaaaggagtcacacactccacctggagacgccttgaagtaactgcacgaaatttgaggatggccaggcagttctacaacagccgctcacagggagagccagaacacagaagaactcagatgactggtagtattaccttcttcataatcccaggcttggggggctgcgatggagtcagaggaaactcagttcagaacatctttggtttttacaaatacaaattaactggaacgctaaattctagcctgttaatctggtcactgaaaaaaaattttttttttttcaaaaaacatagctttagcttattttttttctctttgtaaaacttcgtgcatgacttcagctttactctttgtcaagacatgccaaagtgctgagtcactaataaaagaaaaaaagaaagtaaaggaagagtggttctgcttcttagcgctagcctcaatgacgacctaagctgcacttttccccctagttgtgtcttgccatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctcattctgccctcgagcccaccgggaacgaaagagaagctctatctcccctccaggagcccagctatgaactccttctccacaagtaagtgcaggaaatccttagccctggaactgccagcggcggtcgagccctgtgtgagggaggggtgtgtggcccagggagggctggcgggcggccagcagcagaggcaggctcccagctgtgctgtcagctcacccctgcgctcgctcccctccggcacaggcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggaggtgggtaggcttggcgatggggttgaagggcccggtgcgcatgcgttccccttgcccctgcgtgtggccgggggctgcctgcattaggaggtctttgctgggttctagagcactgtagatttgaggccaacggggccgactagactgacttctgtatttatcctttgctggtgtcaggaagttcctttcctttctggaaaatgcagaatgggtctgaaatccatgcccacctttggcatgagctgagggttattgcttctcagggcttccttttccctttccaaaaaattaggtctgtgaagctcctttttgtcccccgggctttggaaggactagaaaagtgccacctgaaaggcatgttcagcttctcagagcagttgcagtactttttggttatgtaaactcaatggctaggattcctcaaagccattccagctaagattcatacctcagagcccaccaaagtggcaaatcataaataggttaaagcatctccccactttcaatgcaaggtattttggtcctgtttggtagaaagaaaagaacacaggaggggagattgggagcccacactcgaattctggttctgccaaaccagccttgtgatcttgggtaaattccctaccacctctggactccatcagtaaaattgggcgtggactaggtgatctcatagatccttcctgctggaacattctatggcttgaattatattctcctaattattgtcaaaattgctgttattaagtatctactgtgtgccaggcactttaaataaatattgtgtctaatcttcaaaacaaatttgcaaggaaggtttttggagataaggaaactgagactcaggattaagtaacacacctaaagtcacaggtgagcttggaactgaacccaagtgtgcccccactccactggaatttgcttgccaggatgccaatgagttgtagcttcatttttcttagagactttcctggctgtggttgaacaatgaaaaggccctctagtggtgtttgttttagggacacttaggtgataacaattctggtattctttcccagacatgtaacaagagtaacatgtgtgaaagcagcaaagaggcactggcagaaaacaacctgaaccttccaaagatggctgaaaaagatggatgcttccaatctggattcaatgaggtaccaacttgtcgcactcacttttcactattccttaggcaaaacttctccctcttgcatgcagtgcctgtatacatatagatccaggcagcaacaaaaagtgggtaaatgtaaagaatgttatgtaaatttcatgaggaggccaacttcaagcttttttaaaggcagtttattcttggacaggtatggccagagatggtgccactgtggtgagattttaacaactgtcaaatgtttaaaactcccacaggtttaattagttcatcctgggaaaggtactctcagggccttttccctctctggctgcccctggcagggtccaggtctgccctccctccctgcccagctcattctccacagtgagataacctgcactgtcttctgattattttataaaaggaggttccagcccagcattaacaagggcaagagtgcaggaagaacatcaagggggacaatcagagaaggatccccattgccacattctagcatctgttgggctttggataaaactaattacatggggcctctgattgtccagttatttaaaatggtgctgtccaatgtcccaaaacatgctgcctaagaggtacttgaagttctctagaggagcagagggaaaagatgtcgaactgtggcaattttaacttttcaaattgattctatctcctggcgataaccaattttcccaccatctttcctcttaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaataactgtattttaaactatatattaactgaggtggattttaacatcaatttttaatagtgcaagagatttaaaaccaaaggcgggggggcgggcagaaaaaagtgcatccaactccagccagtgatccacagaaacaaagaccaaggagcacaaaatgattttaagattttagtcattgccaagtgacattcttctcactgtggttgtttcaattctttttcctaccttttaccagagagttagttcagagaaatggtcagagactcaagggtggaaagaggtaccaaaggctttggccaccagtagctggctattcagacagcagggagtagacttgctggctagcatgtggaggagccaaagctcaataagaaggggcctagaatgaaacccttggtgctgatcctgcctctgccatttctacttaagccagggtttctcatatgttaacatgcatgggaattccctgggcatcttcttgtggtgtggagtctgacttagcaagcctcgggtgggtttgagggtcaaatttctaccaggcttatatccctggtgatgctgcagaattccaggaccacacttggaggtttaaggccttccacaagttacttatcccatatggtgggtctatggaaaggtgtttcccagtcctctttacaccaccggatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgctggtcaggcttggtggctcatgcctgtattccctaaactttgggagcctaaggtgagaactccttgaactcctaaggccaggagttcaagaccagcctggataacatagcaagaccccatctctacaaaaaaccaaaaccaaacaaacaaaaatgatagtggtgcttccctcaggatgcttgtggtctaatgggagacagaacagcaaagggatgattagaagttggttgctgtgagccaggcacagtgctgatataatcccagcgctatgggaggctgaggtgggtggatcatttgaggccaggagtttaagaccagcctggtcaacatggtaaaaccccatctctacttaaaaatacaaaaaagttagccaggcatggtggcatacacctgtaacccagctactcaggaggctgaggcacatgaatcacttgaacccaggaggcagaggttgctgtgcaccactgcactccagcctgggtgacagaacgagaccttgactcaaaaaaaaaaaaaagaagtttgttgctatggaagggtcctactcagagcaggcaccccagttaatctcattcaccccacatttcacatttgaacatcatcccatagcccagagcatccctccactgcaaaggatttattcaacatttaaacaatcctttttactttcattttccttcaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaactttgagtgtgtcacgtgaagcttaatataaacaagtttcttgtcactgccaccaccacgaccaaaaaaagctaatcaatcactatatataatacatatatatactatatataataaatatatatactatatataatacatatatacactatatataatacatatatactatatatacacatatatactatatatacacatatatattatgaatgtatatatatagtatatatagtatatatactatgtatgtatatatatagtatatatagtatatatactatgtatgtgtatatatagtatatatagtatatatagtatatatactatgtatgtatatatatagtatatatagtatatatactgtgtatgtatatatatagtatatatatactatatatgcatacatagtatatatgcatatatactatatatactatatatttatatatactatatactatatatactatatactgtatatatactatatatgtatgtatacgatatatatatatactatatatgtacacacacacatatatatatacatataagcacctactacatgccaggcatcattaaatgtgttgcatccatcacgtcatttaaccccagcacttgcacactcctttctggttgtggaagactaagtaatttatctaagtcacccagctggaaggtcaggcagggacccagatttgaaatccaagtctacctacctacaggtcccctactcttaacctgtaggtcccactgcctacccaggaactgagggatgatgtagaaaatcccaaaacatgttaatatagggaatacctataaacatgcaatcaaagtctttgggactatacaaccactgtataaagcataacaatgtacaagcttccaaacaataactagaagttctgcctccctcttctgggttcctaaagcactgcacctatctacctgtcaaagcatctaccacattgtaccacaccttaaaatcaatggtttttttcttctcagccagcatgtggatgcctcaataaagcagactcctttcatgacctaaaactaatttcaggggggaaaaaaagacgagctgggcgcagtggctcacgcctataatcccagcactttgggaggctgaggcgggaggatcacttgaggtcagaagaccagcctggccaacatggcaaaaccccgtctctactaaaaatacaaaaattagctgggcgtggtggcgcacctataatcccagctactcaggaagctgagacatgataatcgcttgagcctgggaggtagagcctggggctgcactccatcctgggcaacagagggagattctgtctcaaaaaataataataataatataaataaataaataatttttttaaaaaaagactctttcctatattaatctttgcatcctgtgcccatggccccaaacctgaccaatgaaggccccagtaaatattttttgaacaaaagaaaatatagatgatcaaagataagaaagataagaagacacctcaattcttgtaggcatatagtggtaggggaaatcactaattctgtgtgtttatgtatcttcatcttcaggaaaaatagcaagaattagaaagaaacactcaaaggacttccagagaaggcaatgaatagccagtgcatgtacacctacctccatttcctttgccactggcaataatcttcatctgccctctcccctccccgacctaaagtggtagtgagagaggcaatctggacaacttaacatggcctcgtttatccatctcaacacgacactgggaatttggtgcagatttcccctttctaacccctgcaatataaaagttctgtctccttataaagcttttccccattctacccgctacagacttcttcctccaggggaggagttcttctgggtaagggaaagtccacagaggtcaaaaaccgagaggagctgagccagaaaagagccacatactctgatttctagaaaccacgcatcacaagtgggcttcccaaaactagggtcaagtcaaagaacaaagagaaattgcgatcaactgatgctttcattcttcaaacaaaaatgggttgagtttcatagataagtcagggaaagctcctgagaaagaggtgaaagggtagacatgatccctgccctcttgtgtagcacactatggaagggaaaatagacattaaataaagactataaatgtgataaggtttccaaaaagaaatgtgggctgtgctgagaacccatactagaggcacggataatctaagtgaaccctggtttggctgctcaccactcgaaagccagacatgagagacaaggttggtgggaggaaaagccagttcatcagaaagccagtaaaccaagaagatggtgagctagtattttaaagtaccatcttgaattttaaaatttatcacagtagtttttaaagagaaacttggtatgggagatacgtgggagtggtgcgtggtgtagggtctgtgtgtcttgttctgatggctatctcaggtaaccgcctgtccggaggtctgtttgggatcatcttaacttcagctagatgatggattcattgttcatgattccttctaaccaggaggattctgcaatgggggctccttgcctcgtttgtttaaagatcagcctctgggatttttaaagcaagagtataattagataagcatacattgccagaggggagtgtctagagagggaaggaatgaagaggtgagaggaaagaaaaggaagaaaaagaaagtgggtaagaggcagagcaagatggcagaatagaaagctccaccaatggaccccctggcaaggatacaaagttaacaactatctgcacagaaaaacaaacaaacaaaaaacaccttcataagaatcagaactcaggtgagcacacatagtacctggttttaacttcatatcactgaaacaggcactgaagaaattaaaaaacagtcctgaatcacaatgctacccctcccctatccacagcagcagtggtgtggtgcggagaatgtctctgggtgctgggagaaggagaacacatcaattgtggggcactgaactcagtactgtcctgttagaggagaaaggaaaaccagactaaacttagctgatgtccactcacagagggagcatttaaaccatccctagcccaagaggaattgtcaatccccagcagtccaaacttgagtagccgcaaaactctccactgagggccaaacttgaaaggcagtctaggccataaggactgcaactcttaggcaagtactagggctcaacaaggcccggagacctagtggactgaggggacatgtgacataccgagataccagctgaggcagccaagggagtattggcttcacccctctcctaatcctaggctgcacaagtcatagcttcaaaagagactttttctttctgcttgaggagagaagacagaagagtggggaggacttggccttacctcttggataccagctcagctacagcaggatagggcactagtcagagtcataaggcccgtattccagacccaagctc

SEQ ID NO: 12 - DNA, 108 bp, Artificial Sequence
  5′ end junction of Neo cassette and human IL6
aggcccgtattccagacccaagctcgtcgacctgcagccaagctatcgaattcctgcagcccaattccgatcatattcaataacccttaatataacttcgtataatgt

SEQ ID NO: 13 - DNA, 80 bp, Artificial Sequence
  3′ end junction of Neo cassette with mouse IL6
ctatacgaagttattaggtccctcgaggggatccactagtcttacccaacatgagcaaggtcctaagttacatccaaaca

SEQ ID NO: 14 - DNA, 22 bp, Artificial Sequence
  primer
tgcatcgcattgtctgagtagg

SEQ ID NO: 15 - DNA, 23 bp, Artificial Sequence
  primer
acttaggaccttgctcatgttgg

SEQ ID NO: 16 - DNA, 20 bp, Artificial Sequence
  primer
gctcgactagagcttgcgga

SEQ ID NO: 17 - DNA, 23 bp, Artificial Sequence
  primer
cagaagcctgatatcttagtgtc

SEQ ID NO: 18 - DNA, 21 bp, Artificial Sequence
  primer
ccatggaaggagttacagaga

SEQ ID NO: 19 - DNA, 23 bp, Artificial Sequence
  primer
gtactgaggcatataaagtttgc

SEQ ID NO: 20 - DNA, 19 bp, Artificial Sequence
  primer
gggaccactatggttgaat

SEQ ID NO: 21 - DNA, 23 bp, Artificial Sequence
  primer
cagaagcctgatatcttagtgtc

SEQ ID NO: 22 - DNA, 23 bp, Artificial Sequence
  sgRNA target sequence
agtctcaatagctccgccagagg

SEQ ID NO: 23 - DNA, 23 bp, Artificial Sequence
  sgRNA target sequence
gtctataccacttcacaagtcgg

SEQ ID NO: 24 - DNA, 23 bp, Artificial Sequence
  sgRNA target sequence
gggcgcctgctgctagctgatgg

SEQ ID NO: 25 - DNA, 23 bp, Artificial Sequence
  sgRNA target sequence
tgctggccaacccacaatgctgg

SEQ ID NO: 26 - DNA, 23 bp, Artificial Sequence
  sgRNA target sequence
agtctcctgcgtggagaaaaggg

SEQ ID NO: 27 - DNA, 23 bp, Artificial Sequence
  sgRNA target sequence
tgtgctatctgctcacttgccgg

SEQ ID NO: 28 - DNA, 23 bp, Artificial Sequence
  sgRNA target sequence
gccttcacttacttgcagagagg

SEQ ID NO: 29 - DNA, 23 bp, Artificial Sequence
  sgRNA target sequence
atgcttaggcataacgcactagg

SEQ ID NO: 30 - DNA, 23 bp, Artificial Sequence
  sgRNA target sequence
gtccacaaactgatatgcttagg

SEQ ID NO: 31 - DNA, 23 bp, Artificial Sequence
  sgRNA target sequence
tgcctaagcatatcagtttgtgg

SEQ ID NO: 32 - DNA, 23 bp, Artificial Sequence
  sgRNA target sequence
aagtcactttgagatctactcgg

SEQ ID NO: 33 - DNA, 23 bp, Artificial Sequence
  sgRNA target sequence
taagtcagatacctgacaacagg

SEQ ID NO: 34 - DNA, 23 bp, Artificial Sequence
  sgRNA target sequence
tattctgttacctagccagatgg

SEQ ID NO: 35 - DNA, 23 bp, Artificial Sequence
  sgRNA target sequence
ttccaagaaaccatctggctagg

SEQ ID NO: 36 - DNA, 23 bp, Artificial Sequence
  sgRNA target sequence
gaactgacaatatgaatgttggg

SEQ ID NO: 37 - DNA, 132 bp, Artificial Sequence
  sgRNA scaffold
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 38 - DNA, 20 bp, Artificial Sequence
  sgRNA sequence
agtctcctgcgtggagaaaa

SEQ ID NO: 39 - DNA, 24 bp, Artificial Sequence
  sgRNA sequence
taggagtctcctgcgtggagaaaa

SEQ ID NO: 40 - DNA, 20 bp, Artificial Sequence
  sgRNA sequence
ttttctccacgcaggagact

SEQ ID NO: 41 - DNA, 24 bp, Artificial Sequence
  sgRNA sequence
aaacttttctccacgcaggagact

SEQ ID NO: 42 - DNA, 20 bp, Artificial Sequence
  sgRNA sequence
tattctgttacctagccaga

SEQ ID NO: 43 - DNA, 24 bp, Artificial Sequence
  sgRNA sequence
taggtattctgttacctagccaga

SEQ ID NO: 44 - DNA, 20 bp, Artificial Sequence
  sgRNA sequence
tctggctaggtaacagaata

SEQ ID NO: 45 - DNA, 24 bp, Artificial Sequence
  sgRNA sequence
aaactctggctaggtaacagaata

SEQ ID NO: 46 - DNA, 1573 bp, Artificial Sequence
  5′ homologous arm
aatctactctaatcgcctgtgtgtttacactgggttacattctttagagtgtacttatattctccttttgcattctcaatataaattaatctgctagatataaagctgttctctttattttagtgtaatttttttcttcacattgaattctaggagaaactatgctagtgatatataattcttgaactattaaacatgggagcataagaaaacaagaatcttaaggcaatctgcagagtgaagaagctgattgtgatcctgagagtgtgttttgtaaatggttttggattttatgtacagagcctactttcagcctggaatcattctgaatgctagctagatatctggagacaggtggacagaaaaccaggaactagtctgaaaaagaaactaaccaaagggaagaagtctgtttaagtttgacccagcctagaagacttgagcattggaggggttattcagagtgagacgtaccaccttcagattcaaatcctgtcatccagtagaagggagcttcaaacacaagctagctaagatacaatgaggtccttcttcgatatctttatcttccatataccatgaatcaaagaaacttcaacaacatgaggactgcaacagaccttcaagcctccttgcatgacctggaaatgttttggggtgtcctggcagcagtgggatcagcactaacagataagggcaactctcacagagactaaaggtcttaactaagaagatagccaagagaccactggggagaatgcagagaataggcttggacttggaagccaagattgcttgacaacagacagaagatatttctgtacttcacccactttacccacctggcaactcctggaaacaactgcacaaaatttggaggtgaacaaaccattagaaacaactggtcctgacaagacacaggaaaaacaagcaatatgcaacattactgtctgttgtccaggttgggtgctgggggtgggagagggagtgtgtgtctttgtatgatctgaaaaaactcaggtcagaacatctgtagatccttacagacatacaaaagaatcctagcctcttattcatgtgtgtgtgtgtgtgtgtgtgtgtgtgtgtgtgtgtgtgtgtgtgtatgtgtgtgtcgtctgtcatgcgcgcgtgcctgcgtttaaataacatcagctttagcttctctttctccttataaaacattgtgaatttcagttttctttcccatcaagacatgctcaagtgctgagtcacttttaaagaaaaaaaagaagagtgctcatgcttcttagggctagcctcaaggatgacttaagcacactttccccttcctagttgtgattctttcgatgctaaacgacgtcacattgtgcaatcttaataaggtttccaatcagccccacccactctggccccacccccaccctccaacaaagatttttatcaaatgtgggattttcccatgagtctcaaaattagagagttgactcctaataaatatgagactggggatgtctgtagctcattctgctctggagcccaccaagaacgatagtcaattccagaaaccgct

SEQ ID NO: 47 - DNA, 1328 bp, Artificial Sequence
  3′ homologous arm
agtgactgaaagacgcatctcagctggtaaagttcttacccaacatgagcaaggtcctaagttacatccaaacatcctcccccaaatcaataattaagcactttttatgacatgtaaagttaaataagaagtgaaagctgcagatggtgagtgagagatgccatgagaaagcattgcatataccacattagttaatttcaggtcttgtacattcttttctggacatgagagagtaagggatctaactaagccaccttttggaaacataaaacataatctctgatttgaattcaagtctacctccctctaggtccatttttaacttttagttgtaatttgaagacagatatagaaaaatctcaaaacattttaatatgaattatacacttagagttgatgtcacagattctgagaccatgggactacttagataagatatagctccaaaagataaaagcgccaaaataatatccagaagttctgcctccctcgtctggagtctccatgcactgcatacctcctattagtgtctgccattatatatcataccttaaaactgaaggagctttctatccaactagcatatgggtccctcaagaaagcagactctagtgttttaaccttttcgtgctatatataggtaaggagcctgaacaaaggagacccctataagtatttgctgaatgaaaagagaatagttaatcacagtataacaaaagtcagttcttggtaaatacagagcatttgggtgacattacagtgatgtgttattgtcttttaaaaaaagtagaaaagaatggaaatgaaacattttaaggatttctaaataaggggcagatacaagagtattttgggttttagcccagactatactgtagggggaaagcctgtctcaactttatcccaatttcatatatgtatgtccatatatgtgcatgtatgtgcacatgcacacatgggtgtgtatgtgtgtgtgtatggtgattatgatgatgatgaagagatgatgtctataacttaatgtggttcttcctatttctgtacaaaactgagaatttggtgccaattctctctctctctctctctctctctctctctctctctctctctctctctctctctctctctctctctttttcttcttcttcttcttcttcttcttcttcttcttcttcttcttcttcttcttcttcttcttcttcttcttcttcttcttcttcttcttcttcttcttctttttattattattttatgtacattccagccattgcccccaggatcccctcccacagttcctcatcccattcctctttccccttgcctccgag

SEQ ID NO: 48 - DNA, 4756 bp, Artificial Sequence
  fragment of human DNA
atgaactccttctccacaagtaagtgcaggaaatccttagccctggaactgccagcggcggtcgagccctgtgtgagggaggggtgtgtggcccagggagggctggcgggcggccagcagcagaggcaggctcccagctgtgctgtcagctcacccctgcgctcgctcccctccggcacaggcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggaggtgggtaggcttggcgatggggttgaagggcccggtgcgcatgcgttccccttgcccctgcgtgtggccgggggctgcctgcattaggaggtctttgctgggttctagagcactgtagatttgaggccaacggggccgactagactgacttctgtatttatcctttgctggtgtcaggaagttcctttcctttctggaaaatgcagaatgggtctgaaatccatgcccacctttggcatgagctgagggttattgcttctcagggcttccttttccctttccaaaaaattaggtctgtgaagctcctttttgtcccccgggctttggaaggactagaaaagtgccacctgaaaggcatgttcagcttctcagagcagttgcagtactttttggttatgtaaactcaatggctaggattcctcaaagccattccagctaagattcatacctcagagcccaccaaagtggcaaatcataaataggttaaagcatctccccactttcaatgcaaggtattttggtcctgtttggtagaaagaaaagaacacaggaggggagattgggagcccacactcgaattctggttctgccaaaccagccttgtgatcttgggtaaattccctaccacctctggactccatcagtaaaattgggcgtggactaggtgatctcatagatccttcctgctggaacattctatggcttgaattatattctcctaattattgtcaaaattgctgttattaagtatctactgtgtgccaggcactttaaataaatattgtgtctaatcttcaaaacaaatttgcaaggaaggtttttggagataaggaaactgagactcaggattaagtaacacacctaaagtcacaggtgagcttggaactgaacccaagtgtgcccccactccactggaatttgcttgccaggatgccaatgagttgtagcttcatttttcttagagactttcctggctgtggttgaacaatgaaaaggccctctagtggtgtttgttttagggacacttaggtgataacaattctggtattctttcccagacatgtaacaagagtaacatgtgtgaaagcagcaaagaggcactggcagaaaacaacctgaaccttccaaagatggctgaaaaagatggatgcttccaatctggattcaatgaggtaccaacttgtcgcactcacttttcactattccttaggcaaaacttctccctcttgcatgcagtgcctgtatacatatagatccaggcagcaacaaaaagtgggtaaatgtaaagaatgttatgtaaatttcatgaggaggccaacttcaagcttttttaaaggcagtttattcttggacaggtatggccagagatggtgccactgtggtgagattttaacaactgtcaaatgtttaaaactcccacaggtttaattagttcatcctgggaaaggtactctcagggccttttccctctctggctgcccctggcagggtccaggtctgccctccctccctgcccagctcattctccacagtgagataacctgcactgtcttctgattattttataaaaggaggttccagcccagcattaacaagggcaagagtgcaggaagaacatcaagggggacaatcagagaaggatccccattgccacattctagcatctgttgggctttggataaaactaattacatggggcctctgattgtccagttatttaaaatggtgctgtccaatgtcccaaaacatgctgcctaagaggtacttgaagttctctagaggagcagagggaaaagatgtcgaactgtggcaattttaacttttcaaattgattctatctcctggcgataaccaattttcccaccatctttcctcttaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaataactgtattttaaactatatattaactgaggtggattttaacatcaatttttaatagtgcaagagatttaaaaccaaaggcgggggggcgggcagaaaaaagtgcatccaactccagccagtgatccacagaaacaaagaccaaggagcacaaaatgattttaagattttagtcattgccaagtgacattcttctcactgtggttgtttcaattctttttcctaccttttaccagagagttagttcagagaaatggtcagagactcaagggtggaaagaggtaccaaaggctttggccaccagtagctggctattcagacagcagggagtagacttgctggctagcatgtggaggagccaaagctcaataagaaggggcctagaatgaaacccttggtgctgatcctgcctctgccatttctacttaagccagggtttctcatatgttaacatgcatgggaattccctgggcatcttcttgtggtgtggagtctgacttagcaagcctcgggtgggtttgagggtcaaatttctaccaggcttatatccctggtgatgctgcagaattccaggaccacacttggaggtttaaggccttccacaagttacttatcccatatggtgggtctatggaaaggtgtttcccagtcctctttacaccaccggatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgctggtcaggcttggtggctcatgcctgtattccctaaactttgggagcctaaggtgagaactccttgaactcctaaggccaggagttcaagaccagcctggataacatagcaagaccccatctctacaaaaaaccaaaaccaaacaaacaaaaatgatagtggtgcttccctcaggatgcttgtggtctaatgggagacagaacagcaaagggatgattagaagttggttgctgtgagccaggcacagtgctgatataatcccagcgctatgggaggctgaggtgggtggatcatttgaggccaggagtttaagaccagcctggtcaacatggtaaaaccccatctctacttaaaaatacaaaaaagttagccaggcatggtggcatacacctgtaacccagctactcaggaggctgaggcacatgaatcacttgaacccaggaggcagaggttgctgtgcaccactgcactccagcctgggtgacagaacgagaccttgactcaaaaaaaaaaaaaagaagtttgttgctatggaagggtcctactcagagcaggcaccccagttaatctcattcaccccacatttcacatttgaacatcatcccatagcccagagcatccctccactgcaaaggatttattcaacatttaaacaatcctttttactttcattttccttcaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaactttgagtgtgtcacg

SEQ ID NO: 49 - DNA, 1154 bp, Artificial Sequence
  humanized mouse IL6
aaatatgagactggggatgtctgtagctcattctgctctggagcccaccaagaacgatagtcaattccagaaaccgctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaa

SEQ ID NO: 50 - DNA, 926 bp, Artificial Sequence
  humanized mouse IL6
aaatatgagactggggatgtctgtagctcattctgctctggagcccaccaagaacgatagtcaattccagaaaccgctatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaa

SEQ ID NO: 51 - DNA, 26 bp, Artificial Sequence
  primer
cggtgaaagaatggtggactcacttc

SEQ ID NO: 52 - DNA, 25 bp, Artificial Sequence
  primer
tgcagaagagagccaaccaaccaaa

SEQ ID NO: 53 - DNA, 24 bp, Artificial Sequence
  primer
ccctgcccagctcattctccacag

SEQ ID NO: 54 - DNA, 24 bp, Artificial Sequence
  primer
ccagagactgagccaccaatgagg

SEQ ID NO: 55 - DNA, 25 bp, Artificial Sequence
  primer
aacagctagcaatggagttgggctt

SEQ ID NO: 56 - DNA, 25 bp, Artificial Sequence
  primer
aaaggtgctttttaagtcgggagca

SEQ ID NO: 57 - DNA, 25 bp, Artificial Sequence
  primer
aggtgagcttggaactgaacccaag

SEQ ID NO: 58 - DNA, 25 bp, Artificial Sequence
  primer
tacccactttttgttgctgcctgga

SEQ ID NO: 59 - DNA, 3377 bp, Mus musculus
cacaccgatctgagccacgccggggcgagcgctcgcagtgcgagctgagtgtggagcccgaggccgagggcgactgctctcgctgccccagtctgccggccgcccggccccggctgcggagccgctctgccgcccgccgtcccgcgtagaaggaagcatgctgaccgtcggctgcacgctgttggtcgccctgctggccgcgcccgcggtcgcgctggtcctcgggagctgccgcgcgctggaggtggcaaatggcacagtgacaagcctgccaggggccaccgttaccctgatttgccccgggaaggaagcagcaggcaatgttaccattcactgggtgtactctggctcacaaaacagagaatggactaccacaggaaacacactggttctgagggacgtgcagctcagcgacactggggactatttatgctccctgaatgatcacctggtggggactgtgcccttgctggtggatgttcccccagaggagcccaagctctcctgcttccggaagaacccccttgtcaacgccatctgtgagtggcgtccgagcagcaccccctctccaaccacgaaggctgtgctgtttgcaaagaaaatcaacaccaccaacgggaagagtgacttccaggtgccctgtcagtattctcagcagctgaaaagcttctcctgccaggtggagatcctggagggtgacaaagtataccacatagtgtcactgtgcgttgcaaacagtgtgggaagcaagtccagccacaacgaagcgtttcacagcttaaaaatggtgcagccggatccacctgccaaccttgtggtatcagccatacctggaaggccgcgctggctcaaagtcagctggcagcaccctgagacctgggacccgagttactacttgctgcagttccagcttcgataccgacctgtatggtcaaaggagttcacggtgttgctgctcccggtggcccagtaccaatgcgtcatccatgatgccttgcgaggagtgaagcacgtggtccaggtccgtgggaaggaggagcttgaccttggccagtggagcgaatggtccccagaggtcacgggcactccttggatagcagagcccaggaccaccccggcaggaatcctctggaaccccacacaggtctctgttgaagactctgccaaccacgaggatcagtacgaaagttctacagaagcaacgagtgtcctcgccccagtgcaagaatcctcgtccatgtccctgcccacattcctggtagctggaggaagcttggcgtttgggttgcttctctgtgtcttcatcatcctgagactcaagcagaaatggaagtcagaggctgagaaggaaagcaagacgacctctcctccacccccaccgtattccttgggcccactgaagccgaccttccttctggttcctctcctcaccccacacagctctgggtctgacaataccgtaaaccacagctgcctgggtgtcagggacgcacagagcccttatgacaacagcaacagagactacttattccccagataatcatctggatggtacctggcagctggcagggcaccacgagatcagcacacaagtttctcatgcgggtcccatccacctggggtggggtggggcgggcggggctgcagcttcactaacccacaagagctctgcacaggttctgagtaggtgcagctggtgctgcataggctctgaaggaaggaaggggctgtgaggaacacaggccattgtgaagacagcttgtgatgactgaatagagatgcccgtcagctccacatctgatagtggctcacaagctgcaccctcaggaggcctcagaaaggggctccaaaggctgccccagctgcctcgctctgcctcactgccccaagccaccttttagctctcgaactcctaaagtccaagcactttgccattctctttccgaggccactgaggccgggtggaagcttggttccgatttccttctcaacatctggaaagcagctgggcccggtggtggtgactaatatctcagggcctgatggtttacgcgagtgacaatttctcacaagcagtttttaaatgtgaatgatgaccccaggcactgctggctgcggaggcttcattttcctcttcgatctcaggacttcaggcgaaaagcggagtggaagtagagagcggatgggtgtccaccgtcctcatggtacttgcgggaggtacagcctggaaaacacgtttcctgtccccctactctcccaggagagggatgatggtagggggtgcctcttccagggcggagagaactactttaccccagccttgcccattctgatttcaactggactggagctactaggaaagtcgacattcatgcaaaaagaaaaaacgttaactagcaagaatgcactttcattttggtttttagagaactgttgcctgtttctctcaagagtctggaagaggccgctcactgcacactactgtatgaaccctcactgcccaccctggaggaccaagtgcagtaacggtagcccaaacaccaagtcaagtgaaaatcgagggaaaaaaaaaacaaacaagcaacaaaaaaaaaaaaccaaaactaaactaaaaaacaaatcacccccccaaaaaaaaacaaaaccaaaaaccaaaaaaaacaaaaaaacaaaacaacaacaacaaaaaaaacccaaaccaacccgctgtttcctataacagaaaagcctttggtttcattttttattttgatttttttgtcttaaaaagtataaaaatagcctgtccatgctctgcttcagggaatgagcctgtgaacactcccaggcgcaggcaggaagggtgtctgcttcctgctacacctcactgccaccttggccttccttgctttacgtttgactgagtggcctcagatgctttcccctggggctttgaggaatccagtgatgttagtggtcaccgaggagaccacagagccacagtgtggtgcttagattaaagtgacttctgcaaccacagcaccccacacctgccgtcttactgaactatgccagtaacttgccttttctgccaccaccacgagacgagacgggcagagctcggaagctgtcaccccatgccctctgcttgtccgctctaggggccactgacctaagcattagttattttattttattttatttttttgtgggttttgtacattttaggtcctgttgctgtcttagaaaaggctctgtaggttgacagaaaatcaggccaagtattcatgttttgttttttttttttttccttctttcctcctttgctaagtttttgggactcaagggtagcaaaactgctgtgaaagggaaatttattaaaaatgttacagatcgtg

SEQ ID NO: 60 - protein, 460 aa, Mus musculus
MLTVGCTLLVALLAAPAVALVLGSCRALEVANGTVTSLPGATVTLICPGKEAAGNVTIHWVYSGSQNREWTTTGNTLVLRDVQLSDTGDYLCSLNDHLVGTVPLLVDVPPEEPKLSCFRKNPLVNAICEWRPSSTPSPTTKAVLFAKKINTTNGKSDFQVPCQYSQQLKSFSCQVEILEGDKVYHIVSLCVANSVGSKSSHNEAFHSLKMVQPDPPANLVVSAIPGRPRWLKVSWQHPETWDPSYYLLQFQLRYRPVWSKEFTVLLLPVAQYQCVIHDALRGVKHVVQVRGKEELDLGQWSEWSPEVTGTPWIAEPRTTPAGILWNPTQVSVEDSANHEDQYESSTEATSVLAPVQESSSMSLPTFLVAGGSLAFGLLLCVFIILRLKQKWKSEAEKESKTTSPPPPPYSLGPLKPTFLLVPLLTPHSSGSDNTVNHSCLGVRDAQSPYDNSNRDYLFPR

SEQ ID NO: 61 - DNA, 5928 bp, Homo sapiens
ggcggtcccctgttctccccgctcaggtgcggcgctgtggcaggaagccaccccctcggtcggccggtgcgcggggctgttgcgccatccgctccggctttcgtaaccgcaccctgggacggcccagagacgctccagcgcgagttcctcaaatgttttcctgcgttgccaggaccgtccgccgctctgagtcatgtgcgagtgggaagtcgcactgacactgagccgggccagagggagaggagccgagcgcggcgcggggccgagggactcgcagtgtgtgtagagagccgggctcctgcggatgggggctgcccccggggcctgagcccgcctgcccgcccaccgccccgccccgcccctgccacccctgccgcccggttcccattagcctgtccgcctctgcgggaccatggagtggtagccgaggaggaagcatgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagctggctgggtggcaccagcagcctggaccctgtggatgataaaacacaaacgggctcagcaaaagatgcttctcactgccatgccagcttatctcaggggtgtgcggcctttggcttcacggaagagccttgcggaaggttctacgccaggggaaaatcagcctgctccagctgttcagctggttgaggtttcaaacctccctttccaaatgcccagcttaaaggggctagagtgaacttgggccactgtgaagagaaccatatcaagactctttggacactcacacggacactcaaaagctgggcaggttggtgggggcctcggtgtggagaagcggctggcagcccacccctcaacacctctgcacaagctgcaccctcaggcaggtgggatggatttccagccaaagcctcctccagccgccatgctcctggcccactgcatcgtttcatcttccaactcaaactcttaaaacccaagtgccttagcaaattctgtttttctaggcctggggacggcttttacttaaaccgccaaggctgggggaagaagctctctcctccctttcttccctacagttgaaaaacagctgagggtgagtgggtgaataatacagtatctcagggcctggtcgttttcaacagaattataattagttcctcattagcattttgctaaatgtgaatgatgatcctaggcatttgctgaatacagaggcaactgcattggctttgggttgcaggacctcaggtgagaagcagaggaaggagaggagaggggcacagggtctctaccatcccctgtagagtgggagctgagtgggggatcacagcctctgaaaaccaatgttctctcttctccacctcccacaaaggagagctagcagcagggagggcttctgccatttctgagatcaaaacggttttactgcagctttgtttgttgtcagctgaacctgggtaactagggaagataatattaaggaagacaatgtgaaaagaaaaatgagcctggcaagaatgtgtttaaacttggtttttaaaaaactgctgactgttttctcttgagagggtggaatatccaatattcgctgtgtcagcatagaagtaacttacttaggtgtgggggaagcaccataactttgtttagcccaaaaccaagtcaagtgaaaaaggaggaagagaaaaaatattttcctgccaggcatggtggcccacgcacttcgggaggtcgaggcaggaggatcacttgagtccagaagtttgagatcagcctgggcaatgtgataaaaccccatctctacaaaaagcataaaaattagccaagtgtggtagagtgtgcctgaagtcccagatacttggggggctgaggtgggaggatctcttgagcctgggaggtcaaggctgcagtgagccgagattgcaccactgcactccagcctgggtgacagagcaagtgagaccctgtctcaaaaaaagaaaaagaaaaagaaaaaatattttccctattagagaagagattgtggtttcattctgtattttgtttttgtcttaaaaagtggaaaaatagcctgcctcttctctactctagggaaaaaccagcgtgtgactactcccccaggtggttatggagagggtgtccggtccctgtcccagtgccgagaaggaagcctcccacgactgcccggcagggtcctagaaattccccaccctgaaagccctgagctttctgctatcaaagaggttttaaaaaaatcccatttaaaaaaaatcccttacctcggtgccttcctctttttatttagttccttgagttgattcagctctgcaagaattgaagcaggactaaatgtctagttgtaacaccatgattaaccacttcagctgacttttctgtccgagctttgaaaattcagtggtgttagtggttacccagttagctctcaagttatcagggtattccagagtggggatatgatttaaatcagccgtgtaaccatggacccaatatttaccagaccacaaaacttttctaatactctaccctcttagaaaaaccaccaccatcaccagacaggtgcgaaaggatgaaagtgaccatgttttgtttacggttttccaggtttaagctgttactgtcttcagtaagccgtgattttcattgctgggcttgtctgtagattttagaccctattgctgcttgaggcaactcatcttaggttggcaaaaaggcaggatggccgggcgcggtggctcacgcctgtaatcctagcactttgggaggccaaggtgggaggattgcttgagctcaggagtttgagaccaacctgggtaacatagtgagacaccatctctattatgaacaataacagttaagaaaaaaaaaggcaggcaggcggttatggtggttccctcccatcccaccacataaagtttctgagacttgagaacagcaaaatgctgttaaagggaaatattaagaatgagaatctgcagtaagggtgattctgtgcccacagttcttcaattctttataccgttttacccacatgtggtgttaccaaagccgggcagaaccatgctagcggaagatgtgaaatccagatagctcattattgccaagagctaggcagctttgatctccaaattgttattgctttcatttttattgtaatggaattgctttgttttgtttttttgtttttgtattgaagagggttgttttccctttatttttcataagctaatgtaaatgaagaaaaaatgtcttctctgggctgtaggcctggctcagcgtacacaggtatacatcctaagctctctatgttctctaatctgtggtgactgaacatgtgtctcaatgcacggggcatttctacctgtgtttctgcagcacccccactgccttgagtccccagcagtgctgttatttgcctaacacctgtagccatctgccacgcagccagacgtgaaacgctgagacagagaccatttaggttaaatacgacagcttatcctgctgggtggggaaagtaaaaaatatgctggttcaaggcctaaagtaaaatgatcaataatgtttgtagcattaatgaaatattttcaagaaatgtgtccaggggtagcactggctatgttgacgaggcctttggtaactcagagagctcttggccctgatggggacttgcccttacgctttctttatcaggctctgagttcacacggagcctctggcacttccctgctgtcttgggagaaaggaaactggttgccgcggcaggttgtggaatctgttgctggaaccaggctggaagcccacctggtagtgaacagggcccagtggggcaggctgggcatgttgtggtctatgggtttgtttcctggagaatgttcaggaatgtcttcccagctgctttggtgctgagctctattatctcacagcacgtccagaaggctaacccaggtggggaggatgctgacaccagctccaggtggagttggtggtcttaatttggagatgcaggggcaacctgtgaccctttgaggcaagagccctgcacccagctgtcccgtgcagccgtgggcaggggctgcacacggaggggcaggcgggccagttcagggtccgtgccaggccctcctcagtgccctgtgaaggcctcctgtcctccgtgcggctgggcaccagcaccagggagtttctatggcaaccttagtgattattaaggaacactgtcagttttatgaacatatgctcaaatgaaattctactttaggaggaaaggattggaacagcatgtcacaaggctgttaattaacagagagaccttattggatggagatcacatctgttaaatagaatacctcaactctacgttgttttcttggagataaataatagtttcaagtttttgtttgtttgttttacctaattacctgaaagcaaataccaaaggctgatgtctgtatatggggcaaagggtcagtatatttttcagtgtttttttttctaccagctattttgcatttaaagtgaacattgtgtttggaataaatactcttaaaaaataaaaaaaaaaaaaaa

SEQ ID NO: 62 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 63 - DNA, 4133 bp, Artificial Sequence
  5′ homologous arm
gtgaagtcctgtgttcctttgctgcagtcacacgggcttgtctgaattccctgcacagggtactttagcctcacctgccgttcatttctgcttcctggaggccagatgtctccattcccaagttgagatttatctgatacactgttacaccaataggagggtcttttcctggagttggcaggcacccaggaggggctggtaactcctggggacaggtagcaggagtctgacttggggtgggaggttcatggggtgggggttgtcagcaaagctcatacacacaggtttgatttctctcctccttaacagtgttgtcgtaacaacctggaatgttggctggaaaactgaacttccactaagtgtagagttcagacagcctccaaggaggaaagagactgctcagagccctgaggaatgtctttccttaccttagatagagatcttcagtatgtgttgggcccttcagcaggagatgcagagatgagttcttcagggtgacagggaaggcagggtggtaaacaaagtcacatggcatttgggaggaacacaggactctggcacccaagctaaccagtggatgacagctttcttggaattgaggaaagagaccattgagttttctgaaaaagacagccaagtccaggtttggcttttttttttttttttttttttttttaacttgtggattacgttgggcccacacttggggtaattttatctttccttcctggatactgaggatacatgtgttcacagctatgcttggctttttttttttttaatagggtcttgtccagcgtggccagaaccttgccacgtttcccccttagcctcttcagtgccagaattacaatcatccaccaccgtgcctgtcagagacgagtgtgagggcagagtagggtgtaagactaaaggaaaggaaggcctggccagaggataagcttgaacaaggcatgcagattagaaaccacggacatcattgtagaaacttacctacacgcaaggctctggtacaggaaagagggtgggaaagtgaaggtggactctctgtgtagccctggatgacctggaactcactctgtggaccaggctggccttaaactcacagagatatgcctgcttctgcctcccaagtgctgggattaaaggcaggagccatcataccaggacttgaagcacaacactaatactttgtatattataaattcttaccacacaaatcagtaaaaaaggctctgagatgaagtagaaacttgagttctttggaaagtcagttgtgtggcatggtattttgctggggcaaacatgtgacatgaaggagtattttcctgaagcagacacaaggtaaaaggatgttttgctaaagcagacccgtgaaaggacccatgatgtaggattcttctctaactacatgcaaggatcagttcaccttacatttcattgttgagctccatttgttgtgactccatagagagaaacatactggaaacgtttctagtggtgttctggtggcttcttgctgcttccatggactcgggcagagtgatgtcagctgatacagactcaagtggagttttgctaagacagactcacatactgaggcgagacccatttgagaacacgttatgtttggagagcggataactaagactcaatggacagtgggaaagggcttgcttgcatagctagctttgcaatgcttcctggtcttcttcgctgatcttcactttgttaagagaagtaaggcaaagaacttctccaggcctaatcactcctgctaactcatgcagattcggccgaggcctggctgtttctgctaggttgtgccaacactgctgttgatttgtgtttgttatcctgacactaccgacctggactgctgctatattcatcaagtatttgcaagtggatgggatttgctccaaagaacaatttctagacagatccacctcctaatatcctaactacctaattcctgtatcctaataactttccatttccactgcctctaggagggaggttaaagtgtttaaaaaccatcattaaaattagggttgagaaatatctaaacaacacagagaaattaactataacttgcccaaatcctacagctatcaagcagtagacctgggatgggaactaaactcatctgactatggaacacccccccccccacactccaggatcaaaacggggtggatggggggaagccacttggccagggttgccgggtcgtgtctgagatgtctgctcttgccttcaacattccttctatctgagctggcagccttctgtagccttcctgtttcaggccagcaacttgtctctctgcaattattttcaggtctctgtttaccacagaaaggaaggaggcagcacaatgagagtctgttggaaattaattatatttttaagatagggcaagttccactatgcttgcctcgaactctggatcttcctgcctcagcctccagagttttgggattacaggtgtgtactaccacatcctcttgctatttaatcccattttaaatagcaggagaaaaaaatatgtattttttttagacagggccttagactcacagctattcacctgcttctgcttcccaaatgcctgccccaccacagggagagagaacaaccatgcctttctctccgacttatagcgtggctgaggaagtgccatcaaaaccaatcccattgtcctgggtacaggtttggttctgacatccacagagagcggtggatgcctaggtctgaggccagcatgttgcttaaataaggtcatgtcttatttaggggttcagccagcacacacacattccagttgccccatggactctaatcagctgaaccctgaaatggaagaatacattaaaacatactggtgtaggtccagggctccccatctacattttcttttctttcttttttttttcttatcccttcccttctccctcttgctccggcccggctcgctcaggcccataggttttatttgtttctcattacagatggttgtgagctaccatgttgctgggatttgaactcatgatctccggaagtcagtcagtgcgtttaaccgctgagccatctcaccagccctaaattttcttttcagtgtgtttctttccctctgcatttgcatgggaagctgccctgtagcttcaagctctgtagcttggaagggaaccttggtggagtctggtaaatagcaaacgccggttcctttgtgtccagtcaagtcatgatgggtaatcaaagccctactccttgttgagcagttaggctccaggtacatcgacagtgtttccaggaatctttatcatgggcttaaaaatacatctctaataggtgagaaaactgagacttccggatcgggatagcaatcttgaggagggtaacacatgtgtctctgggccagactcaaaagcgcacactggtttcgttgcctctgcagtagccttcaaagagctgctcctccactggaagatgagaggaaatctttttctcaggttatggacgcctaagttttccttcagaccaattaaatcacaatctctgggctcgaagcagacatcggtgtcttttcggattccctgtttgattcccgtaagcacccaggacactgacctgccttctactttaacagccagagccactggctggcgctcatgaaagcaaaactcaagcggaggcgcggatggaaggaaggagggggatatttctccttatctcagcgtcaaaacctggggaggggccctggtgggggtccctgggggctcaggtgtggcgcggctgcaggaagtaaccccctccggtggcccggagcgctggccgattgcgccatcctactgggctttcgtaaccgcgagccggggctgccaagaggagcttcagcaccggttcctcaaatgtttcactgttgccaggacggccggctgctgtgtgtcacgtgcatgtgggaagtcgcgcccacaccgatctgagccacgccggggcgagcgctcgcagtgcgagctgagtgtggagcccgaggccgagggcgactgctctcgctgccccagtctgccggccgcccggccccggctgcggagccgctctgccgcccgccgtcccgcgtagaaggaagc

SEQ ID NO: 64 - DNA, 4727 bp, Artificial Sequence
  3′ homologous arm
gctgcacgctgttggtcgccctgctggccgcgcccgcggtcgcgctggtcctcgggagctgccgcgcgctgggtaagaggaacccggagcgggcgtggggaatgtgacactgtctggttccgttcgcagagtgagccccgccggtaccggctgctagcctgggctgactcacctgtgctgacgcggtggaggttcgcgcgccccctgttgcgtctagcgcctataactgcgtgctcctgtctgtgtctggccagttacctcaagtctgagtacaggacagtgtgttttcctgaaacctgactctggtgtctggttcccagtgttcccagtcttactcaactgcatcccgggtcgctccaaaaagagatacccagagggcgcgccctagtggaacagtagtagtcccttctggagagtcctttatgccactggctctctctgctttagttgtggcacctcgcgtggctttccttagttctactacattccatgtcgtgtgtaatgacagtgacccgacggtgaagggtctgacatcatgggctctgagtcatgggtgttcccgcctcacaaacttactcctttagccaactagccctagcatgcagaatgccaacacccacatccatcccaatcctgtgagtttgacagcaaaggatgtgggacctcttggaactgtgcctccgccccctgccccagccctccctgattagcaggtggaccaggcggagggtagggtaggggcactgtgtttaccttccttgcactctgccttgtaggtctaggcctctctgtgggctctggcttcaggcccagcagcctgcctggtctcttctggaggaactgggagactgcttctctacaggttattttaggggctgagggcttattcaacaacatagaaagagccaaaggacatgggggagatttatcctgtgctggtcgtggggacagtggcttccaggctttctttctgcctggaaggtagaattcctcaagcttggtttgttacccctacctatattttctttatatatatatatataaagaaaatatatatatatattagacaggctctcccataacccaggctggcctcaaacttgtgcctcttctgcctctatctcttgagtgctgggatttcaggtattaacccaccctgtattttacacgtttttaatcctctacaggattgacataaagctttgggaggagcctgaggttcagagagatgcaatgggaagcatcaggcccctccgtgtctcagtgttctcctgtgggtacagctgagggcgtctggggtaggctgagcaccactatcatctggtgggattctgggggagaagacactgatgaaagagaagatccccgcttagctgttcatggtgccaggcaggactgaggtcctctcttccatcttgggagagcaggactgggcctctctgcctgtctcagttactactcaacataccccagcgcccccaccaccacttccctttgttcctaaggagctgcaaggcacttcggagacagcgagttctgttttcctgtggttcagaaaggacacccaagggtgggggtgtcttctgacccaaacagcctttagaaggcagaactgggagtctggggaggaagtgaaagccctgccagtttagaaggaagaggcagagaagtccccagcccatctcccacacccacacttgcacagctacaaactggagagtgacaatcaagaaacaagcaccaaacaggaagtgtggagcaaatgactgagatgtttggctttagagaagtgaggccagatggagatggctctctgggggggaacccatgggtggaagatatacataaacatatatatatgggcaggcgagatagctcagcacacgcacacacacacacacacacacacacacacacactctcctccatccatcatccttagtgggtttttgccttcatgtattcatgtctgtgtgagggttccaaatcctatggaactggagttacagttgtgagctgccatgtggtgcagggattgaacctgggtcctctggaagaacagccagtgttcttaactgctgagccatcactccagcaccaacaggtgggattttcatagtctgcccctccagggcctgctaggactcctgtgtcttcttccacctggggcccaggaccagctgggactgaggcttctgccaggtcaaactttctaggcgctgctgactgaggacaggctgggcatgggtggcttcagtccccctttccttctgtaaggcaagaaaacacgcagcagcaccgatgcctatgtatctaaatctcctcctgacttctgaattccctctgggacaacctctttcggtctacaaaacctacctgtattcctagcctcaccctgaatcagtatttttctgaaggatatattttttatctagcatcaaatttcggctaggaaggatagatctttgtaaaaaaaaaatcacaatttttaaaacattagggtttgtgggttttttgtttgtttgttttttttttttttttgatattttgagacaggtgtttctttgtgtagccctggctgccctggaactcactctgtagaccaggttggccttgaacttagaggtctttgcctcctgagtgctgggatcaaaggccacggtacccaaccaaaaatgacaattcttgacccctggccactcattaaacatgtatcagaagagcagagagtgaggtctacactttgaaaaaaaacaaattgttccttgtggcccctgggcactggcttgaggacatttgcctaatggtcaggatgagtaaaagaccttcttcccctgccaggttccctgtgaccagaagcctctccccgccttggctctgcccctccccttgtttattcgttgcaacacatatttatgtattccctgtaaagcccttggggaccaagtgggatccgtcatgcacactccaccctataaaggatcctgaatgtggccaaaagcaaatcctgagacagagccagttcagtctctctgacctgacactgggtaccaactctccagggacagattgtctcagcagtcagctagacagaggaacccttctgactgggaccctaaagcaacaggagcctgcctgttctctaccccatctgactggcatcaaactgtgcaggcgcaagtgtggccatagctgggttatgtaaacaaagtaacttcagagcttctggcttcaaacagatgtctccttcagcaagctttccatctgcccctgcaggcctgagtgccaccactcagtcatcagcttgaatccagggctaaagctatctcagatgagccagtaagtgagtttaagctattaaaccttaaattaatagaaaaagtaaaatgggcacatgagatggctcagcagataaaaaaaggcatttgctgccgacagggctgatgagctagggacccggcggggggggggggtgcggggggggggggaggtgttactcccaagggtttctgtttctctggcctctacatatgtactgtggctagtgcatgcacataaacaaacaaacaaataaatgtaattaaaacaaaagctgtgtatggtggcacatgcccttagtgccagaatctctgtgagtttgtggtcagcctggtctccacagcaagttcaagaaagaggaaaaaagggtgatttctagatgtgcttgatggtgtaactcatatgggaacttgatgcagggggattgcaagctctaggttagcctaagctacagagacagggttggggtgggggtggggagtaggggaaccctcaaaaaaaaacacaaacaaacaaacaaaaaccaggacaaatctctgtccttcatcacgcctgtcacttgcagtgcgcagtggctagctgtggctggtgtggctgtgcatgtctgctcatggggctttcccatagcggaggggaagtgtgtcgggtgaggggctagagagctatgcggtgtcttccagacacctgttccacctgtatgcatggtgctctgagggggcattggagcctctggaactggagttacagatggttgtgagctgtcttgtgggtgctggggatcaaatcctgcttcattgaaagaactctctctttttggagacaaagtctcaccacgtagccctggctggcttggaactctctgtgtagaacaggctggccccgaatgcacagagagcctcccgcctctgtctctcaagtgatggaactaaaggtgtgggctcaggaggtcatcagtgacggtttatcagtgagctgtcatctttctagtcccagagctgacgctttacctgaacctcagcagctttcaccattgcgcttgtctcttgccagctctgtctgcaggtttctagcacagggtgctcctgaggccccaacccaggagcccgttctctgactcacttctctccctacctccttccagaaaatcattctttgggcttccctttctgtggtggttacagtaactgtggctctgagtatcaacttcatttgaaaaattgtgtcagacccttggcacaacagtgcagctttgggtgagtggggtgccagcccgctcttggctggacagggtagctacaggacagcaggtggggttggatgagtgatcagaggccattctggggcatgtgggaactgagacatctatagcacaaccaagaaaggatgctaaatcaggtctggtggactgc

SEQ ID NO: 65 - DNA, 4098 bp, Artificial Sequence
  IL-6R-A fragment
atgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccagtgcaagattcttcttcagtaccactgcccacattcctggttgctggagggagcctggccttcggaacgctcctctgcattgccattgttctgaggttcaagaagacgtggaagctgcgggctctgaaggaaggcaagacaagcatgcatccgccgtactctttggggcagctggtcccggagaggcctcgacccaccccagtgcttgttcctctcatctccccaccggtgtcccccagcagcctggggtctgacaatacctcgagccacaaccgaccagatgccagggacccacggagcccttatgacatcagcaatacagactacttcttccccagatagagatctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagaattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttcatcagtcaggtacataatggtggatccagtact

SEQ ID NO: 66 - DNA, 80 bp, Artificial Sequence
  5′ end junction of Neo cassette and WPRE-PA
gggggaggattgggaagacaatagcaggcatgctggggagaattccgaagttcctattctctagaaagtataggaacttc

SEQ ID NO: 67 - DNA, 80 bp, Artificial Sequence
  3′ end junction of Neo cassette and mouse IL6R
tctctagaaagtataggaacttcatcagtcaggtacataatggtggatccagtactgctgcacgctgttggtcgccctgc

SEQ ID NO: 68 - DNA, 25 bp, Artificial Sequence
  primer
ataaggtttccaatcagccccaccc

SEQ ID NO: 69 - DNA, 25 bp, Artificial Sequence
  primer
acttaggaccttgctcatgttgggt

SEQ ID NO: 70 - DNA, 23 bp, Artificial Sequence
  primer
agcgcacgtctgccgcgctgttc

SEQ ID NO: 71 - DNA, 25 bp, Artificial Sequence
  primer
tgcctgtaggtgactctcaagtcca

SEQ ID NO: 72 - DNA, 25 bp, Artificial Sequence
  primer
ctgggattccacatctgttgtccac

SEQ ID NO: 73 - DNA, 25 bp, Artificial Sequence
  primer
acagtggcattgtcttccggctcta

SEQ ID NO: 74 - DNA, 25 bp, Artificial Sequence
  primer
ctgggattccacatctgttgtccac

SEQ ID NO: 75 - DNA, 22 bp, Artificial Sequence
  primer
tgcagctaccgttcatgtcccc

SEQ ID NO: 76 - DNA, 25 bp, Artificial Sequence
  primer
gtcaacaagcacaactcttccaggg

SEQ ID NO: 77 - DNA, 24 bp, Artificial Sequence
  primer
ccagaggcttctaaaccctaaagc

SEQ ID NO: 78 - DNA, 21 bp, Artificial Sequence
  primer
ggatcggccattgaacaagat

SEQ ID NO: 79 - DNA, 22 bp, Artificial Sequence
  primer
cagaagaactcgtcaagaaggc

SEQ ID NO: 80 - DNA, 25 bp, Artificial Sequence
  primer
aaatgtttcactgttgccaggacgg

SEQ ID NO: 81 - DNA, 25 bp, Artificial Sequence
  primer
gacacagacaggagcacgcagttat

SEQ ID NO: 82 - DNA, 25 bp, Artificial Sequence
  primer
cagtggcattgtcttccggctctac

SEQ ID NO: 83 - DNA, 23 bp, Artificial Sequence
  primer
gcatcgataccgtcgacctcgac

SEQ ID NO: 84 - DNA, 25 bp, Artificial Sequence
  primer
gacacagacaggagcacgcagttat

SEQ ID NO: 85 - DNA, 25 bp, Artificial Sequence
  primer
gacaagcgttagtaggcacatatac

SEQ ID NO: 86 - DNA, 24 bp, Artificial Sequence
  primer
gctccaatttcccacaacattagt

SEQ ID NO: 87 - DNA, 589 bp, Artificial Sequence
  WPRE sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

(19) United States
(12) Patent Application Publication
(10) Pub. No.: US 2022/0015343 A1
(43) Pub. Date: Jan. 20, 2022

(54) GENETICALLY MODIFIED NON-HUMAN ANIMAL WITH HUMAN OR CHIMERIC GENES

(71) Applicants: Biocytogen Pharmaceuticals (Beijing) Co., Ltd., Beijing (CN); Biocytogen Jiangsu Co., Ltd., Jiangsu (CN)

(72) Inventors: Yuelei Shen, Beijing (CN); Meiling Zhang, Beijing (CN); Jiawei Yao, Beijing (CN); Chaoshe Guo, Beijing (CN); Yanan Guo, Beijing (CN); Yang Bai, Beijing (CN); Rui Huang, Beijing (CN); Lei Zhao, Beijing (CN); Chengzhang Shang, Beijing (CN)

(21) Appl. No.: 17/293,279

(22) PCT Filed: Dec. 17, 2019
(86) PCT No.: PCT/CN2019/126045

§ 371 Date: May 12, 2021

(30) Foreign Application Priority Data
Dec. 17, 2018 (CN) 201811543170.9

(51) Int. Cl. A01K 67/027; C07K 14/715; C12N 15/85; C07K 14/54

(52) U.S. Cl. CPC A01K 67/0278; C07K 14/7155; C12N 15/8509; C07K 14/5412; A01K 2217/072; C12N 2830/36; A01K 2267/0325; A01K 2207/15; C12N 2015/8563; C12N 2830/60; A01K 2227/105

(57) ABSTRACT

The present disclosure relates to genetically modified non-human animals that express a human or chimeric (e.g., humanized) IL6R and/or IL6, and methods of use thereof.

### CLAIM OF PRIORITY

This application claims the benefit of Chinese Patent Application App. No. 201811543170.9, filed on Dec. 17, 2018. The entire contents of the foregoing are incorporated herein by reference.

### TECHNICAL FIELD

This disclosure relates to genetically modified animal expressing human or chimeric (e.g., humanized) genes, and methods of use thereof.

### BACKGROUND

Interleukin-6 (IL6) is a cytokine produced by several different cell types. It acts as a pro-inflammatory cytokine and an anti-inflammatory myokine. There is substantial evidence showing that targeting IL6/IL6R pathway can be a therapeutic strategy for treating immune-related disorders (e.g., allergy and autoimmune diseases) in humans.

The traditional drug research and development for therapeutic agents that target IL6/IL6R pathway typically use in vitro screening approaches. However, these screening approaches are still different from what happens in the in vivo environment (such as cell microenvironment, extracellular matrix components and immune cell interaction, etc.), resulting in a high rate of failure in drug development. There is a need for humanized animal models that are suitable for human antibody screening and efficacy evaluation.

### SUMMARY

This disclosure is related to an animal model with human IL6R and/or IL6 or chimeric IL6R and/or IL6. The animal model can express human IL6R and/or IL6 or chimeric IL6R and/or IL6 (e.g., humanized IL6R and/or IL6) protein in its body. It can be used in the studies on the function of IL6R and/or IL6 gene, and can be used in the screening and evaluation of anti-human IL6R and anti-IL6 antibodies. In addition, the animal models prepared by the methods described herein can be used in drug screening, pharmacodynamics studies, treatments for immune-related diseases (e.g., autoimmune disease, allergies). They can also be used to facilitate the development and design of new drugs, and save time and cost. In summary, this disclosure provides a powerful tool for studying the function of IL6R and/or IL6 protein and a platform for screening treatments for immune-related diseases.

In one aspect, the disclosure is related to a genetically-modified, non-human animal whose genome includes at least one chromosome comprising a sequence encoding a human or chimeric interleukin-6 receptor (IL6R).

In some embodiments, the sequence encoding the human or chimeric IL6R is operably linked to an endogenous regulatory element at the endogenous IL6R gene locus in the at least one chromosome.

In some embodiments, the sequence encoding the human or chimeric IL6R is operably linked to a human IL6R regulatory element at the endogenous IL6R gene locus in the at least one chromosome.

In some embodiments, the at least one chromosome includes one or more endogenous IL6R exons, and the one or more endogenous IL6R exons are inactivated.

In some embodiments, the sequence encoding a human or chimeric IL6R includes a sequence encoding an amino acid sequence that is at least 70%, 75%, 80%, 85%, 90%, 95%, 99%, or 100% identical to human IL6R (NP_000556.1; SEQ ID NO: 62).

In some embodiments, the animal includes a sequence that is at least 70%, 75%, 80%, 85%, 90%, 95%, 99%, or 100% identical to SEQ ID NO: 65, SEQ ID NO: 66, or SEQ ID NO: 67 or 1-1407 bp of SEQ ID NO: 65.

In some embodiments, the animal is a mammal, e.g., a monkey, a rodent or a mouse. In some embodiments, the animal is a mouse. In some embodiments, the animal does not express endogenous IL6R. In some embodiments, the animal expresses a decreased level of IL6R (e.g., endogenous IL6R) as compared to IL6R expression level in a wild-type animal.

In some embodiments, the animal has one or more cells expressing human or chimeric IL6R.

In some embodiments, the animal has one or more cells expressing human or chimeric IL6R, and the expressed human or chimeric IL6R can bind to endogenous IL6.

In some embodiments, the animal has one or more cells expressing human or chimeric IL6R, and the expressed human or chimeric IL6R can bind to human IL6.

In one aspect, the disclosure is related to a genetically-modified, non-human animal,

In some embodiments, the genome of the animal includes an insertion of a sequence encoding a human or chimeric IL6R at an endogenous IL6R gene locus.

In some embodiments, the sequence encoding the human or chimeric IL6R is operably linked to an endogenous regulatory element at the endogenous IL6R locus, and one or more cells of the animal express a human or chimeric IL6R.

In some embodiments, the sequence encoding the human or chimeric IL6R is operably linked to a human regulatory element at the endogenous IL6R locus, and one or more cells of the animal express a human or chimeric IL6R.

In some embodiments, the animal does not express endogenous IL6R. In some embodiments, the animal expresses a decreased level of IL6R (e.g., endogenous IL6R) as compared to IL6R expression level in a wild-type animal.

In some embodiments, a sequence encoding an amino acid sequence that is at least 70%, 75%, 80%, 85%, 90%, 95%, 99%, or 100% identical to human IL6R (SEQ ID NO: 62) is inserted at the endogenous IL6R locus.

In some embodiments, the inserted sequence further includes Woodchuck Hepatitis Virus (WHP) Posttranscriptional Regulatory Element (WPRE) and/or polyA (polyadenylation) signal sequence.

In some embodiments, the animal is heterozygous with respect to the insertion at the endogenous IL6R gene locus. In some embodiments, the animal is homozygous with respect to the insertion at the endogenous IL6R gene locus.

In one aspect, the disclosure is related to a non-human animal including at least one cell comprising a nucleotide sequence encoding a chimeric IL6R polypeptide.

In some embodiments, the chimeric IL6R polypeptide includes at least 50 contiguous amino acid residues that are identical to the corresponding contiguous amino acid sequence of a human IL6R,

In some embodiments, the animal expresses the chimeric IL6R.

In some embodiments, the chimeric IL6R polypeptide includes a sequence that is at least 90%, 95%, or 99% identical of SEQ ID NO: 62.

In some embodiments, the nucleotide sequence is operably linked to an endogenous IL6R regulatory element of the animal.

In some embodiments, the nucleotide sequence is integrated to an endogenous IL6R gene locus of the animal.

In some embodiments, the animal is a NOD-Prkdc^{scid} IL-2rg^{null} mouse.

In some embodiments, the animal further includes a sequence encoding an additional human or chimeric protein (e.g., IL6, IL33, IL13, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or Signal regulatory protein α (SIRPα)).

In some embodiments, the additional human or chimeric protein is IL6.

In one aspect, the disclosure is related to a method for making a genetically-modified, non-human animal, including inserting in at least one cell of the animal, at an endogenous IL6R gene locus, a sequence encoding a human or chimeric IL6R.

In some embodiments, the sequence encoding the human or chimeric IL6R includes one or more exons selected from exon 1, exon 2, exon 3, exon 4, exon 5, exon 6, exon 7, exon 8, exon 9 and exon 10 of a human IL6R gene.

In some embodiments, the sequence encoding the human or chimeric IL6R includes at least 30, 50, 100, 200, or 300 nucleotides of exon 1, exon 2, exon 3, exon 4, exon 5, exon 6, exon 7, exon 8, exon 9 and/or exon 10 of a human IL6R gene.

In some embodiments, the sequence encoding human or chimeric IL6R encodes a sequence that is at least 90% identical to SEQ ID NO: 62.

In some embodiments, the sequence encoding human or chimeric IL6R is under the control of an endogenous IL6R regulatory element.

In some embodiments, the animal is a mouse, and the locus is within exon 1 of the mouse IL6R gene. In some embodiments, the sequence is inserted immediately before the start codon.

In some embodiments, the animal or mouse further includes a sequence encoding an additional human or chimeric protein (e.g., IL6, IL33, PD-1, CTLA-4, LAG-3, BTLA, PD-L1, CD27, CD28, TIGIT, TIM-3, GITR, CD137, OX40, CD47 or SIRPa).

In some embodiments, the additional human or chimeric protein is IL6.

In some embodiments, the animal is a NOD-Prkdc^{scid} IL-2rg^{null} mouse.

In one aspect, the disclosure is related to a genetically-modified, non-human animal whose genome includes at least one chromosome comprising a sequence encoding a human or chimeric IL6.

In some embodiments, the sequence encoding the human or chimeric IL6 is operably linked to an endogenous regulatory element at the endogenous IL6 gene locus in the at least one chromosome.

In some embodiments, the sequence encoding the human or chimeric IL6 is operably linked to a human regulatory element at the endogenous IL6 gene locus in the at least one chromosome.

In some embodiments, the sequence encoding a human or chimeric IL6 includes a sequence encoding an amino acid sequence that is at least 70%, 75%, 80%, 85%, 90%, 95%, 99%, or 100% identical to human IL6 (NP_000591.1 (SEQ ID NO: 6) or NP_001305024.1 (SEQ ID NO: 8)).

In some embodiments, the animal includes a sequence that is at least 70%, 75%, 80%, 85%, 90%, 95%, 99%, or 100% identical to SEQ ID NO: 12, SEQ ID NO: 13, SEQ ID NO: 11 or SEQ ID NO: 48.

In some embodiments, the animal expresses a sequence that is at least 70%, 75%, 80%, 85%, 90%, 95%, 99%, or 100% identical to SEQ ID NO: 49 or SEQ ID NO: 50.

In some embodiments, the animal is a mammal, e.g., a monkey, a rodent or a mouse.

In some embodiments, the animal is a NOD-Prkdc^{scid} IL-2rg^{null} animal.

In some embodiments, the animal does not express endogenous IL6. In some embodiments, the animal expresses a decreased level of IL6 (e.g., endogenous IL6) as compared to IL6 expression level in a wild-type animal.

In some embodiments, the animal has one or more cells expressing human IL6.

In some embodiments, the animal has one or more cells expressing human or chimeric IL6, and the expressed human or chimeric IL6 can bind to endogenous IL6R.

In some embodiments, the animal has one or more cells expressing human or chimeric IL6, and the expressed human or chimeric IL6 can bind to human IL6R.

In one aspect, the disclosure is related to a genetically-modified, non-human animal.

In some embodiments, the genome of the animal includes a replacement of a sequence encoding a region of endogenous IL6 with a sequence encoding a corresponding region of human IL6 at an endogenous IL6 gene locus.

In some embodiments, the sequence encoding the corresponding region of human IL6 is operably linked to an endogenous regulatory element at the endogenous IL6 locus, and one or more cells of the animal expresses a human IL6.

In some embodiments, the sequence encoding the corresponding region of human IL6 is operably linked to a human regulatory element at the endogenous IL6 locus, and one or more cells of the animal expresses a human IL6.

In some embodiments, the animal does not express endogenous IL6. In some embodiments, the animal expresses a decreased level of IL6 (e.g., endogenous IL6) as compared to IL6 expression level in a wild-type animal.

In some embodiments, the replaced locus includes a sequence that is at least 70%, 75%, 80%, 85%, 90%, 95%, 99%, or 100% identical to SEQ ID NO: 12, SEQ ID NO: 13, SEQ ID NO: 49, SEQ ID NO: 50, SEQ ID NO: 11, or SEQ ID NO: 48.

In some embodiments, the animal is a mouse, and the replaced endogenous IL6 region is exon 1, exon 2, exon 3, exon 4 and/or exon 5 of the endogenous mouse IL6 gene.

In some embodiments, the animal is heterozygous with respect to the replacement at the endogenous IL6 gene locus.

In some embodiments, the animal is homozygous with respect to the replacement at the endogenous IL6 gene locus.

In one aspect, the disclosure is related to a non-human animal including at least one cell comprising a nucleotide sequence encoding a human or chimeric IL6 polypeptide,

In some embodiments, the human or chimeric IL6 polypeptide includes at least 50 contiguous amino acid residues that are identical to the corresponding contiguous amino acid sequence of a human IL6.

In some embodiments, the human or chimeric IL6 polypeptide has at least 100 contiguous amino acid residues that are identical to the corresponding contiguous amino acid sequence of a human IL6.

In some embodiments, the nucleotide sequence is operably linked to an endogenous IL6 regulatory element of the animal.

In some embodiments, the nucleotide sequence is operably linked to a human IL6 regulatory element of the animal.

In some embodiments, the nucleotide sequence is integrated to an endogenous IL6 gene locus of the animal.

In some embodiments, the animal further includes a sequence encoding an additional human or chimeric protein (e.g., IL6R, IL33, IL13, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or SIRPa).

In some embodiments, the additional human or chimeric protein is IL6R.

In some embodiments, the animal is a NOD-Prkdc^{scid} IL-2rg^{null} mouse.

In one aspect, the disclosure is related to a method for making a genetically-modified, non-human animal, including replacing in at least one cell of the animal, at an endogenous IL6 gene locus, a sequence encoding a region of an endogenous IL6 with a sequence encoding a corresponding region of human IL6.

In some embodiments, the sequence encoding the corresponding region of human IL6 includes exon 1, exon 2, exon 3, exon 4 and/or exon 5 of a human IL6 gene.

In some embodiments, the sequence encoding the corresponding region of IL6 includes at least 100, 150 or 200 nucleotides of exon 1, exon 2, exon 3, exon 4 and/or exon 5 of a human IL6 gene.

In some embodiments, the sequence encoding the corresponding region of human IL6 encodes a sequence that is at least 90% identical to SEQ ID NO: 6 or 8.

In some embodiments, replaced locus includes a sequence that is at least 70%, 75%, 80%, 85%, 90%, 95%, 99%, or 100% identical to SEQ ID NO: 12, SEQ ID NO: 13, SEQ ID NO: 49, SEQ ID NO: 50, SEQ ID NO: 11, or SEQ ID NO: 48.

In some embodiments, the animal is a mouse, and the locus is exon 1, exon 2, exon 3, exon 4 and exon 5 of the mouse IL6 gene.

In one aspect, the disclosure is related to a method of making a genetically-modified mouse cell that expresses a chimeric IL6, the method includes replacing, at an endogenous mouse IL6 gene locus, a nucleotide sequence encoding a region of mouse IL6 with a nucleotide sequence encoding a corresponding region of human TL6, thereby generating a genetically-modified mouse cell that includes a nucleotide sequence that encodes the chimeric IL6, In some embodiments, the mouse cell expresses the chimeric IL6.

In some embodiments, the nucleotide sequence encoding the chimeric IL6 is operably linked to an endogenous IL6 regulatory region, e.g., promoter.

In some embodiments, the nucleotide sequence encoding the chimeric IL6 is operably linked to a human IL6 regulatory region, e.g., promoter.

In some embodiments, the animal or mouse further includes a sequence encoding an additional human or chimeric protein (e.g., IL6R, IL33, IL13, PD-1, CTLA-4, LAG-3, BTLA, PD-L1, CD27, CD28, TIGIT, TIM-3, GITR, CD137, OX40, CD47, or SIRPα).

In some embodiments, the additional human or chimeric protein is IL6R.

In some embodiments, the animal is a NOD-Prkdc^{scid} IL-2rg^{null} mouse.

In one aspect, the disclosure is related to a method of determining effectiveness of an IL6-IL6R pathway inhibitor for treating an immune disorder, including administering the IL6-IL6R pathway inhibitor to the animal as described herein; and determining the inhibitory effects of the IL6-IL6R pathway inhibitor.

In some embodiments, the immune disorder is allergy. In some embodiments, the immune disorder is an autoimmune disorder. In some embodiments, the immune disorder is multiple sclerosis, asthma, allergy, arthritis, or autoimmune encephalomyelitis.

In one aspect, the disclosure is related to a method of determining effectiveness of an IL6-IL6R pathway inhibitor for reducing inflammation, including administering the IL6-IL6R pathway inhibitor to the animal as described herein; and determining the inhibitory effects of the IL6-IL6R pathway inhibitor.

In one aspect, the disclosure is related to a method of determining effectiveness of an IL6-IL6R pathway inhibitor for treating autoimmune disorder, including administering the IL6-IL6R pathway inhibitor to the animal as described herein; and determining the inhibitory effects of the IL6-IL6R pathway inhibitor.

In some embodiments, the autoimmune disorder is multiple sclerosis. In some embodiments, the autoimmune disorder is arthritis.

In some embodiments, the IL6-IL6R pathway inhibitor is an anti-human IL6 antibody. In some embodiments, the IL6-IL6R pathway inhibitor is an anti-human IL6R antibody.

In some embodiments, the inhibitory effects are evaluated by paw thickness and/or an arthritis score.

In some embodiments, the inhibitory effects are evaluated by behavioral scores, brain/spinal cord IHC pathology, serum/brain homogenate Th17 type multi-cytokine detection, and/or CNS and spleen flow cytometry.

In one aspect, the disclosure is related to a method of determining toxicity of an anti-IL6R antibody or an anti-IL6 antibody, the method includes administering the anti-IL6R antibody or the anti-IL6 antibody to the animal as described herein; and determining weight change of the animal.

In some embodiments, the method as described herein further includes performing a blood test (e.g., determining red blood cell count).

In one aspect, the disclosure is related to a genetically-modified, non-human animal.

In some embodiments, the genome of the animal includes a replacement of a sequence encoding a region of endogenous IL6R with a sequence encoding a corresponding region of human IL6R at an endogenous IL6R gene locus.

In some embodiments, the sequence encoding the corresponding region of human IL6R is operably linked to an endogenous regulatory element at the endogenous IL6R locus, and one or more cells of the animal express a human or chimeric IL6R.

In some embodiments, the animal does not express endogenous IL6R or expresses a decreased level of endogenous IL6R as compared to IL6R expression level in a wild-type animal.

In some embodiments, the animal is a mouse, and the replaced endogenous IL6R region is exon 1, exon 2, exon 3, exon 4, exon 5, exon 6, exon 7, exon 8, exon 9 and/or exon 10 of the endogenous mouse IL6R gene.

In some embodiments, the animal is heterozygous with respect to the replacement at the endogenous IL6R gene locus. In some embodiments, the animal is homozygous with respect to the replacement at the endogenous IL6R gene locus.

In one aspect, the disclosure is related to a method for making a genetically-modified, non-human animal, including replacing in at least one cell of the animal, at an endogenous IL6R gene locus, a sequence encoding a region of an endogenous IL6R with a sequence encoding a corresponding region of human IL6R.

In some embodiments, the sequence encoding the corresponding region of human IL6R includes exon 1, exon 2, exon 3, exon 4, exon 5, exon 6, exon 7, exon 8, exon 9 and/or exon 10 of a human IL6R gene.

In some embodiments, the sequence encoding the corresponding region of IL6R includes at least 30, 50, 100, 200, or 300 nucleotides of exon 1, exon 2, exon 3, exon 4, exon 5, exon 6, exon 7, exon 8, exon 9 and/or exon 10 of a human IL6R gene.

In some embodiments, the sequence encoding the corresponding region of human IL6R encodes a sequence that is at least 90% identical to SEQ ID NO: 62.

In some embodiments, the animal is a mouse, and the locus is exon 1, exon 2, exon 3, exon 4, exon 5, exon 6, exon 7, exon 8, exon 9 and/or exon 10 of the mouse IL6R gene.

In one aspect, the disclosure is related to a non-human animal including at least one cell comprising a nucleotide sequence encoding a chimeric IL6R polypeptide,

In some embodiments, the chimeric IL6R polypeptide includes at least 50 contiguous amino acid residues that are identical to the corresponding contiguous amino acid sequence of a human IL6R.

In some embodiments, the animal expresses the chimeric IL6R.

In some embodiments, the chimeric IL6R polypeptide includes a sequence that is at least 90%, 95%, or 99% identical of SEQ ID NO: 62.

In some embodiments, the nucleotide sequence is operably linked to an endogenous IL6R regulatory element of the animal.

In some embodiments, the nucleotide sequence is integrated to an endogenous IL6R gene locus of the animal.

In one aspect, the disclosure is related to a method of making a genetically-modified mouse cell that expresses a chimeric IL6R, the method including replacing, at an endogenous mouse IL6R gene locus, a nucleotide sequence encoding a region of mouse IL6R with a nucleotide sequence encoding a corresponding region of human IL6R, thereby generating a genetically-modified mouse cell that includes a nucleotide sequence that encodes the chimeric IL6R.

In some embodiments, the mouse cell expresses the chimeric IL6R.

In some embodiments, the nucleotide sequence encoding the chimeric IL6R is operably linked to an endogenous IL6R regulatory region, e.g., promoter.

In some embodiments, the animal further includes a sequence encoding an additional human or chimeric protein (e.g., IL6, IL33, IL13, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or Signal regulatory protein α (SIRPa)).

In some embodiments, the additional human or chimeric protein is IL6.

In some embodiments, the animal or mouse further includes a sequence encoding an additional human or chimeric protein (e.g., IL6, IL33, PD-1, CTLA-4, LAG-3, BTLA, PD-L1, CD27, CD28, TIGIT, TIM-3, GITR, CD137, OX40, CD47 or SIRPa).

In some embodiments, the additional human or chimeric protein is IL6.

In one aspect, the disclosure is related to a nucleic acid including a nucleotide sequence.

In some embodiments, the nucleotide sequence is one of the following: SEQ ID NO: 11, 12, 13, 48, 49, 50, 65, 66, or 67; or a sequence that is at least 90%, 91%, 92%, 93%, 94%, 95%, 96%, 97%, 98%, or 99% identical to SEQ ID NO: 11, 12, 13, 48, 49, 50, 65, 66, or 67.

In one aspect, the disclosure is related to a cell including the nucleic acid as described herein.

In one aspect, the disclosure is related to an animal including the nucleic acid as described herein.

The disclosure also relates to a method for establishing a genetically-modified non-human animal expressing two human or chimeric (e.g., humanized) genes. The method includes the steps of (a) using the method for establishing a IL6R gene humanized animal model to obtain a IL6R gene genetically modified humanized mouse; (b) mating the IL6R gene genetically modified humanized mouse obtained in step (a) with another humanized mouse, and then screening to obtain a double humanized mouse model. In some embodiments, in step (b), the IL6R gene genetically modified humanized mouse obtained in step (a) is mated with an IL6 humanized mouse to obtain a IL6R and IL6 double humanized mouse model.

The disclosure also relates to a method for establishing a genetically-modified non-human animal expressing two human or chimeric (e.g., humanized) genes. The method includes the steps of (a) using the method for establishing a IL6 gene humanized animal model to obtain a IL6 gene genetically modified humanized mouse; (b) mating the IL6 gene genetically modified humanized mouse obtained in step (a) with another humanized mouse, and then screening to obtain a double humanized mouse model. In some embodiments, in step (b), the IL6 gene genetically modified humanized mouse obtained in step (a) is mated with an IL6R humanized mouse to obtain an IL6 and IL6R double humanized mouse model.

The disclosure also relates to non-human mammal generated through the methods as described herein. In some embodiments, the genome thereof contains human gene(s).

In some embodiments, the non-human mammal is a rodent. In some embodiments, the non-human mammal is a mouse. In some embodiments, the non-human mammal expresses human IL6R and/or human IL6.

The disclosure also relates to an offspring of the non-human mammal.

In one aspect, the disclosure relates to a non-human mammal model, characterized in that the non-human mammal model is obtained through the methods as described herein. In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal. The disclosure further relates to the tissue, organ or a culture thereof derived from the non-human mammal or an offspring thereof, or the tumor bearing non-human mammal.

In one aspect, the disclosure relates to a tumor tissue derived from the non-human mammal or an offspring thereof when it bears a tumor, or the tumor bearing non-human mammal.

The disclosure further relates to a IL6R and/or IL6 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the non-human mammal, the animal model generated through the method as described herein in the production and utilization of an animal experimental disease model of immunization processes involving human cells, the study on a pathogen, or the development of a new diagnostic strategy and/or a therapeutic strategy.

The disclosure further relates to the use of the non-human mammal or an offspring thereof, or the non-human mammal, the animal model generated through the methods as described herein, in the screening, verifying, evaluating or studying the IL6R and/or IL6 gene function, human IL6R and/or IL6 antibodies, the drugs or efficacies for human IL6R and/or IL6 targeting sites, and the drugs for immune-related diseases.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse IL6 gene locus (based on NM_031168.2).

FIG. 1B is a schematic diagram showing mouse IL6 gene locus (based on NM_001314054.1).

FIG. 1C is a schematic diagram showing human IL6 gene locus (based on NM_000600.4).

FIG. 1D is a schematic diagram showing human IL6 gene locus (based on NM_001318095.1).

FIG. 2 is a schematic diagram showing humanized IL6 gene (replacing coding sequencing, mouse 5′-UTR and mouse 3′-UTR).

FIG. 3 is a schematic diagram showing humanized IL6 gene locus (replacing coding sequence and mouse 3′-UTR).

FIG. 4 is a schematic diagram showing an IL6 gene targeting strategy.

FIG. 5 is a schematic diagram showing the CRE recombination process.

FIG. 6 is a schematic diagram showing an IL6 gene targeting strategy.

FIG. 7A is a histogram showing activity testing results for sgRNA1-sgRNA7. Con is a negative control; PC is a positive control.

FIG. 7B is a histogram showing activity testing results for sgRNA8-sgRNA15. Con is a negative control; PC is a positive control.

FIG. 8A shows tail vein PCR identification results for F0 generation mice, wherein primer pairs L-GT-F1 and L-GT-R were used to amplify the 5′ end targeting site gene fragment. WT is wild-type, H₂O is a blank control, M is the Marker and + is the positive control.

FIG. 8B shows tail vein PCR identification results for F0 generation mice, wherein primer pairs R-GT-F and R-GT-R were used to amplify the 3′ end targeting site gene fragment. WT is wild-type, H₂O is a blank control, M is the Marker and + is the positive control.

FIG. 9A shows tail vein PCR identification results for F1 generation mice (short fragment replacement), wherein primer pairs L-GT-F1 and L-GT-R were used to amplify the 5′ end targeting site gene fragment. WT is wild-type, H₂O is a blank control, M is the Marker and + is the positive control.

FIG. 9B shows tail vein PCR identification results for F1 generation mice, wherein primer pairs R-GT-F and R-GT-R were used to amplify the 3′ end targeting site gene fragment. WT is wild-type, H₂O is a blank control, M is the Marker and + is the positive control.

FIG. 10A is an image showing Southern blot results, wherein F1-026, F1-027, F1-029, F1-030, F1-032, F1-044, F1-045, F1-046, F1-047, F1-050 and F1-052 are labels for the mice.

FIG. 10B is an image showing Southern blot results, wherein F1-022, F1-023, F1-025 and F1-056 are labels for the mice.

FIG. 11A is a histogram showing the ELISA detection results of mouse IL6 protein expression. +/+ represents B-NDG mice and h/+ represents humanized IL6 heterozygous mice with B-NDG background.

FIG. 11B is a histogram showing the ELISA detection results of human IL6 protein expression. +/+ represents B-NDG mice and h/+ represents humanized IL6 heterozygous mice with B-NDG background.

FIG. 12 shows tail vein PCR identification results for gene knockout mice. WT is wild-type, H₂O is a blank control and M is the Marker.

FIG. 13 is a schematic diagram showing mouse and human IL6R gene locus.

FIG. 14 is a schematic diagram showing humanized IL6R gene locus.

FIG. 15 is a schematic diagram showing an IL6R gene targeting strategy.

FIG. 16A is a histogram showing the ELISA detection results of mouse IL6 protein expression. +/+ represents C57BL/6 mice and H/H represents humanized IL6 homozygous mice as shown in FIG. 2.

FIG. 16B is a histogram showing the ELISA detection results of human IL6 protein expression. +/+ represents C57BL/6 mice and H/H represents humanized IL6 homozygous mice as shown in FIG. 2.

FIG. 17 is an image showing Southern blot results. 1-G01 and 1-H01 are cell clone numbers.

FIG. 18 a schematic diagram showing the FLP recombination process.

FIG. 19A shows tail vein PCR identification results for F1 generation IL6R humanized mice, wherein primer pairs IL6R-WT-F and IL6R-WT-R were used to amplify wild-type mouse target site gene fragment. WT is wild-type, H₂O is a blank control, and PC is a positive control.

FIG. 19B shows tail vein PCR identification results for F1 generation IL6R humanized mice, wherein primer pairs IL6R-WT-F and IL6R-Mut-R were used to amplify the target site gene fragment at the 5′ end of the recombinant band. WT is wild-type, H₂O is a blank control, and PC is a positive control.

FIG. 19C shows tail vein PCR identification results for F1 generation IL6R humanized mice, wherein primer pairs IL6R-Frt-F and IL6R-Frt-R were used to amplify the target site gene fragment at the 3′ end of the resistance gene. WT is wild-type, H₂O is a blank control, and PC is a positive control.

FIG. 19D shows tail vein PCR identification results for F1 generation IL6R humanized mice, wherein primer pairs IL6R-Flp-F and IL6R-Flp-R were used in amplification to confirm the presence of Flp. WT is wild-type, H₂O is a blank control, and PC is a positive control.

FIG. 20A is a graph showing the flow cytometry analysis result of wild-type (WT) C57BL/6 mice, wherein cells were stained by mIL-6R PE and mTcRβ-APC/Cy7, to detect IL6R protein expression.

FIG. 20B is a graph showing the flow cytometry analysis result of TL6R humanized homozygous mice (IL6R H/H), wherein cells were stained by mIL-6R PE and mTcRβ-APC/Cy7, to detect IL6R protein expression.

FIG. 20C is a graph showing the flow cytometry analysis result of wild-type (WT) C57BL/6 mice, wherein cells were stained by hTL-6R PE and mTcRβ-APC/Cy7, to detect IL6R protein expression.

FIG. 20D is a graph showing the flow cytometry analysis result of IL6R humanized homozygous mice (IL6R H/H), wherein cells were stained by hIL-6R PE and mTcRβ-APC/Cy7, to detect IL6R protein expression.

FIG. 21A is a histogram showing the ELISA detection results of mouse IL6 protein expression. WT represents wild-type C57BL/6 mice, and IL6^{H/H}IL6R^{H/H} represents double-humanized IL6/IL6R homozygous mice with C57BL/6 background.

FIG. 21B is a histogram showing the ELISA detection results of human IL6 protein expression. WT represents wild-type C57BL/6 mice, and IL6^{H/H}IL6R^{H/H} represents double-humanized IL6/IL6R homozygous mice with C57BL/6 background.

FIG. 22A is a graph showing the flow cytometry analysis result of wild-type (WT) C57BL/6 mice, wherein cells were stained by mIL-6R PE and mTcRβ-APC/Cy7, to detect IL6R protein expression.

FIG. 22B is a graph showing the flow cytometry analysis result of double-humanized IL6/IL6R homozygous mice (IL6^{H/H}IL6R^{H/H}), wherein cells were stained by mIL-6R PE and mTcRβ-APC/Cy7, to detect IL6R protein expression.

FIG. 22C is a graph showing the flow cytometry analysis result of wild-type (WT) C57BL/6 mice, wherein cells were stained by hTL-6R PE and mTcRβ-APC/Cy7, to detect IL6R protein expression.

FIG. 22D is a graph showing the flow cytometry analysis result of double-humanized IL6/IL6R homozygous mice (IL6^{H/H}IL6R^{H/H}), wherein cells were stained by hTL-6R PE and mTcRβ-APC/Cy7, to detect IL6R protein expression.

FIG. 23 shows the alignment between mouse IL6 amino acid sequence (NP_112445.1; SEQ ID NO: 2) and human IL6 amino acid sequence (NP_000591.1; SEQ ID NO: 6).

FIG. 24 shows the alignment between mouse IL6 amino acid sequence (NP_001300983.1 SEQ ID NO: 4) and human IL6 amino acid sequence (NP_001305024.1; SEQ ID NO: 8).

FIG. 25 shows the alignment between mouse IL6R amino acid sequence (NP_034689.2; SEQ ID NO: 60) and human IL6R amino acid sequence (NP_000556.1; SEQ ID NO: 62).

### DETAILED DESCRIPTION

This disclosure relates to transgenic non-human animal with human or chimeric (e.g., humanized) IL6R and/or IL6, and methods of use thereof.

IL6 is a pleiotropic cytokine which is released into the circulation upon injury or infection. IL6 is involved in processes such as hematopoiesis, neural development, inflammation, immunity, reproduction and bone metabolism. In addition, involvement in the induction of B-cell, T-cell and astrocyte differentiation and the induction of acute phase proteins in hepatocytes, such as C-reactive protein (CRP) have been reported. IL6 belongs to the family of IL6-type cytokines that includes IL11, ciliary neurotrophic factor (CTNF), leukemic inhibitory factor (LIF), oncostatin M (OSM) and cardiotrophin-like factor (CLF). All of these cytokines share a four-helix bundle protein motive. This family of proteins signals via receptor complexes which contain glycoprotein 130 (gp130), the common signal transducing protein of the IL6 family of cytokines. Murine IL6 acts in a species-specific manner, whereas human IL6 is also active on IL6R-positive murine cells. Sequence alignments between murine and human IL6 and IL6R indicate that the critical sites of individual amino acid substitutions in human IL6 and IL6R which lead to more than 70% compromised ligand binding affinity (according to Swissprot protein data base). Amino acid identity and similarity between murine and human IL6 are 41.6% and 65%, for the IL6R the corresponding scores are 53.4% and 65.8%. IL6 binds to its receptor (IL6R) and this complex recruits two molecules of gp130, which is ubiquitously expressed, in contrast to IL6R which is expressed on defined cell types such as hepatocytes and leukocytes. A soluble form of the IL6R (sIL6R) can be produced by processing of the receptor by proteases such as a disintegrin and metalloproteinase 17 (ADAM17) or by differential splicing. IL6R by itself is not a signal-transducer, its function is to present IL6 to the signal-transducer gp130. This results in phosphorylation of gp130 by janus kinase 2 (JAK2) and subsequent recruitment of signal transducers and activators of transcription (STAT1 and STAT3) which subsequently dimerize and after phosphorylation they are translocated into the nucleus and mediate transcription of defined gene signatures. This type of signaling is referred to as cis-signaling. sIL6R can bind its ligand IL6 and induce signaling in cells which express gp130 and not IL6R. This kind of signal transduction is referred to as trans-signaling. In contrast to most soluble receptors, the IL6-sIL6R complex can act as an agonist. A soluble fusion protein consisting of the extracellular domain of gp130 and Fc moiety of human IgG has been shown to inhibit trans-signaling due to binding of the IL6-sIL6R complex, whereas cis-signaling was not affected because this fusion protein cannot bind IL6 (WEIDLE, ULRICH H., et al. “Interleukin 6/interleukin 6 receptor interaction and its role as a therapeutic target for treatment of cachexia and cancer.” Cancer Genomics-Proteomics 7.6 (2010): 287-302).

Given that IL6 plays an important role in various disease processes, there are currently three antibody drugs targeting the IL6 pathway in the market, including e.g., ACTEMRA (tocilizumab, targeted IL6R, for treating rheumatoid arthritis, giant cell arteritis, cytokine release syndrome, and idiopathic arthritis in young children and young people), SYLVANT (siltuximab, targeted IL6, for the treatment of Castleman disease) KEVZARA (sarilumab, targeting IL6R, for the treatment of moderate to severe active rheumatoid arthritis in adults).

Experimental animal models are an indispensable research tool for studying the effects of these antibodies before clinical trials. Common experimental animals include mice, rats, guinea pigs, hamsters, rabbits, dogs, monkeys, pigs, fish and so on. However, there are differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models.

Particularly, mouse IL6 protein and human IL6 protein are only about 41% identical, and for IL6R, the percentage identity is only about 53%. Thus, antibodies that recognize human IL6 or IL6R protein cannot recognize mouse IL6 or IL6R. Therefore, during drug development, wildtype mice cannot be used to screen and evaluate the efficacy of drugs targeting human IL6 and IL6R. In addition, while the immunodeficient mice such as NOD-Prkdcscid IL-2rγ^{null} mice are the most suitable tool for transplanting human cells or tissues, it does not work well in human hematopoietic cells. After the transplantation of human hematopoietic cells, there are defects in the development and function of human-derived cells (Watanabe et al. “The analysis of the functions of human B and T cells in humanized NOD/shi-scid/γcnull (NOG) mice (hu-HSC NOG mice).” International immunology 21.7 (2009): 843-858.).

This disclosure provides transgenic non-human animal with human or chimeric (e.g., humanized) IL6R and/or IL6. Because of interaction between human IL6R and human IL6, and also with human hematopoietic cells, the animal model can faithfully mimic the interaction between human IL6R, human IL6, and human hematopoietic cells, and recapitulate effects of IL6 blockade in human patients.

### Interleukin 6 (IL6)

Interleukin 6 (IL6, or IL-6), a pro-inflammatory cytokine, is comprised of 212 amino acids with an N-terminal signal peptide of 29 amino acids and a four-helix bundle arranged in an up-up-down-down topology.

IL6 is a pleiotropic cytokine that mediates acute phase reactions. It is produced not only by immune cells as a mediator of cell proliferation, differentiation, activation and survival, but by various type of parenchymal cells, such as endothelial cells, keratinocytes, adipocytes, and mesangial cells, as an innate response through pattern recognition receptors. It also modulates the production of acute phase proteins, stimulates collagen production from fibroblasts and exerts vascular endothelial activation and osteoclast differentiation.

IL-6 acts on cells through cell membrane gp130 via binding to membrane-bound IL-6 receptor, which is limitedly engaged in leukocytes and hepatocytes. Alternately, IL-6 can act similarly through soluble type IL-6 receptor and gp130. Since gp130 is ubiquitously expressed, IL-6 has the capacity to act on all cells. IL-6 stimulation with adapter protein binding to gp130 results in activation of the JAK/STAT pathway and of the JAK-SH2-domain-containing protein tyrosine phosphatase-2-mitogen-activated protein kinase pathway, leading to cytokine production. This signal transduction is positively regulated by ADAM17 causing shedding of membrane protein-type IL-6 receptor (IL-6R) and by splicing variant soluble (s)IL-6R in circulating microvesicles and is negatively regulated by soluble gp130 and SOCS3.

A detailed description of IL6 and its function can be found, e.g., in Akioka, Shinji. “Interleukin-6 in juvenile idiopathic arthritis.” Modern rheumatology 29.2 (2019): 275-286; Gelinas et al. “Crystal structure of interleukin-6 in complex with a modified nucleic acid ligand.” Journal of Biological Chemistry 289.12 (2014): 8720-8734; each of which is incorporated by reference herein in the entirety.

In human genomes, IL6 gene (Gene ID: 3569) has multiple isoforms or transcripts. Transcript 1 has 5 exons, exon 1, exon 2, exon 3, exon 4 and exon 5. The nucleotide sequence for human transcript 1 mRNA is NM_000600.4 (SEQ ID NO: 5), and the corresponding amino acid sequence is NP_000591.1 (SEQ ID NO: 6). The location for each exon and each region in human IL6 transcript 1 nucleotide sequence and amino acid sequence is listed below:

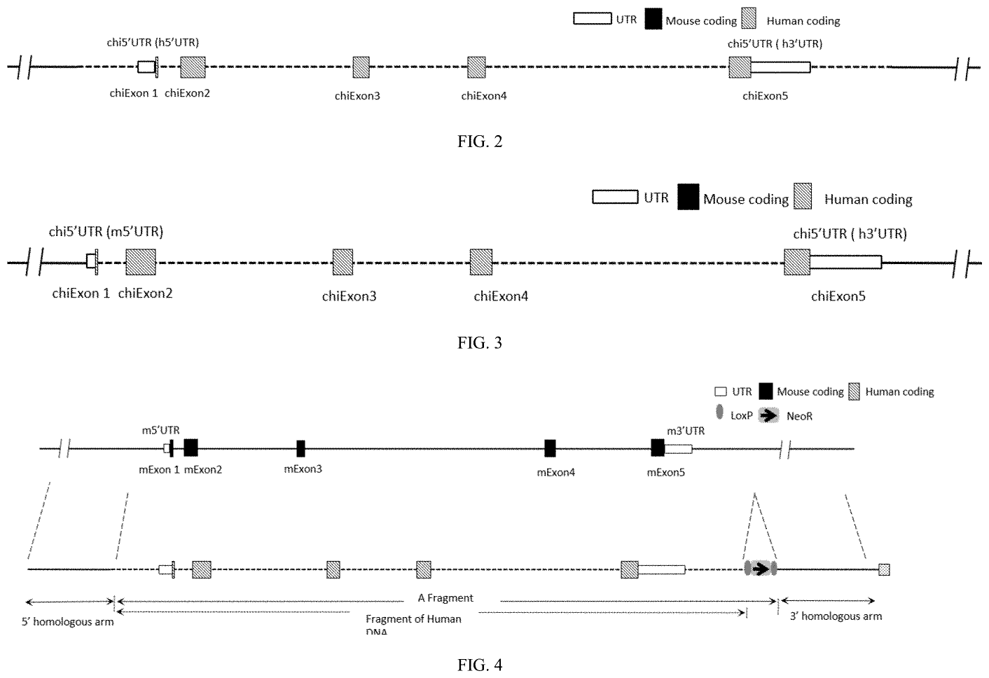

TABLE 1

| | NM_000600.4 | NP_000591.1 |
|---|---|---|
| Human IL6 transcript 1 | 1197bp | 212aa |
| (approximate location) | (SEQ ID NO: 5) | (SEQ ID NO: 6) |
| Exon 1 | 1-140 | 1-6 |
| Exon 2 | 141-331 | 7-70 |
| Exon 3 | 332-445 | 71-108 |
| Exon 4 | 446-592 | 109-157 |
| Exon 5 | 593-1189 | 158-212 |
| Signal peptide | 122-208 | 1-29 |
| Replaced region in | 122-1197 | 1-212 |
| Example (FIG. 3) | | |
| Replaced region in | All | All |
| Example (FIG. 2) | | |

Transcript 2 has 4 exons, exon 1, exon 2, exon 3, and exon 4. The nucleotide sequence for human transcript 2 mRNA is NM_001318095.1 (SEQ ID NO: 7), and the corresponding amino acid sequence is NP_001305024.1 (SEQ ID NO: 8). The location for each exon and each region in human IL6 transcript 2 nucleotide sequence and amino acid sequence is listed below:

TABLE 2

| | NM_001318095.1 | NP_001305024.1 |
|---|---|---|
| Human IL6 transcript 2 | 1006bp | 136aa |
| (approximate location) | (SEQ ID NO: 7) | (SEQ ID NO: 8) |
| Exon 1 | 1-140 | Non-coding |
| Exon 2 | 141-254 | 1-32 |
| Exon 3 | 255-401 | 33-81 |
| Exon 4 | 402-998 | 82-136 |
| Replaced region in | 159-1006 | 1-136 |
| Example (FIG. 3) | | |
| Replaced region in | All | 1-136 |
| Example (FIG. 2) | | |

In mouse genomes, L6 gene (Gene ID: 16193) has multiple isoforms or transcripts. Transcript 1 has 5 exons, exon 1, exon 2, exon 3, exon 4 and exon 5. The nucleotide sequence for mouse transcript 1 mRNA is NM_031168.2 (SEQ ID NO: 1), and the corresponding amino acid sequence is NP_112445.1 (SEQ ID NO: 2). The location for each exon and each region in mouse IL6 transcript 1 nucleotide sequence and amino acid sequence is listed below:

TABLE 3

| | NM_031168.2 | NP_112445.1 |
|---|---|---|
| Mouse IL6 transcript 1 | 1141bp | 211aa |
| (approximate location) | (SEQ ID NO: 1) | (SEQ ID NO: 2) |
| Exon 1 | 1-97 | 1-6 |
| Exon 2 | 98-282 | 7-68 |
| Exon 3 | 283-396 | 69-106 |
| Exon 4 | 397-546 | 107-156 |
| Exon 5 | 547-1141 | 157-211 |
| Signal peptide | 79-150 | 1-24 |
| Replaced region in | 79-1141 | 2-211 |
| Example (FIG. 3) | | |
| Replaced region in | 1-1141 | 1-211 |
| Example (FIG. 2) | | |

Transcript 2 has 5 exons, exon 1, exon 2, exon 3, exon 4 and exon 5. The nucleotide sequence for mouse transcript 2 mRNA is NM_001314054.1 (SEQ ID NO: 3), and the corresponding amino acid sequence is NP_001300983.1 (SEQ ID NO: 4). The location for each exon and each region in mouse IL6 transcript 2 nucleotide sequence and amino acid sequence is listed below:

TABLE 4

| | NM_001314054.1 | NP_001300983.1 |
|---|---|---|
| Mouse IL6 transcript 2 | 1083p | 165aa |
| (approximate location) | SEQ ID NO: 3 | SEQ ID NO: 4 |
| Exon 1 | 1-97 | 1-6 |
| Exon 2 | 98-282 | 7-68 |
| Exon 3 | 283-396 | 69-16 |
| Exon 4 | 397-546 | 107-156 |
| Exon 5 | 547-1083 | 157-165 |
| Signal peptide | 79-150 | 1-24 |
| Replaced region in | 79-1083 | 1-165 |
| Example (FIG. 3) | | |
| Replaced region in | ALL | 1-165 |
| Example (FIG. 2) | | |

The mouse IL6 gene (Gene ID: 16193) is located in Chromosome 5 of the mouse genome, which is located from 30013114 to 30019975 of NC_000071.6 (GRCm38.p4 (GCF_000001635.24)).

The 5′-UTR is from 30,013,114 to 30,013,191, exon 1 is from 30,013,114 to 30,013,210, the first intron is from 30,013,211 to 30,013,375, exon 2 is from 30,013,376 to 30,013,560, the second intron is from 30,013,561 to 30,014,831, exon 3 is from 30,014,832 to 30,014,945, the third intron is from 30,014,946 to 30,018,004, exon 4 is from 30,018,005 to 30,018,154, the fourth intron is from 30,018,155 to 30,019,380, exon 5 is from 30,019,381 to 30,019,975, the 3′-UTR is from 30,019,549 to 30,019,975, based on transcript 1 (NM_031168.2).

The 5′-UTR is from 30,013,168 to 30,013,191, exon 1 is from 30,013,168 to 30,013,210, the first intron is from 30,013,211 to 30,013,375, exon 2 is from 30,013,376 to 30,013,560, the second intron is from 30,013,561 to 30,014,831, exon 3 is from 30,014,832 to 30,014,945, the third intron is from 30,014,946 to 30,018,004, exon 4 is from 30,018,005 to 30,018,154, the fourth intron is from 30,018,155 to 30,019,438, exon 5 is from 30,019,439 to 30,019,597, the 3′-UTR is from 30,019,469 to 30,019,597, based on transcript 2 (NM_001314054.1).

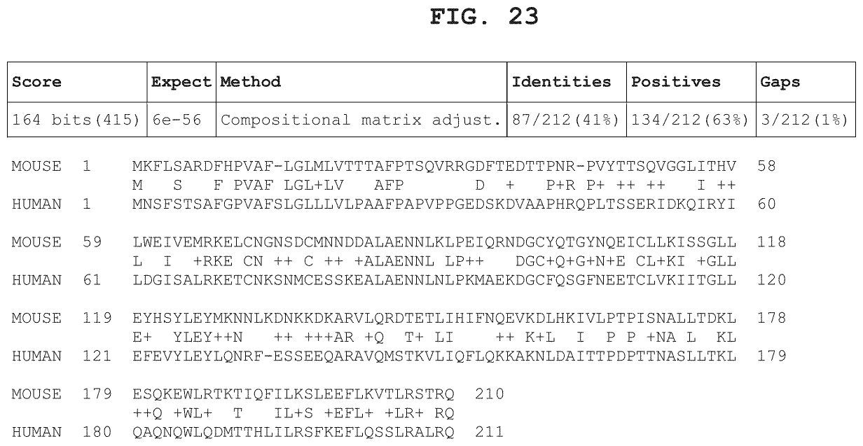

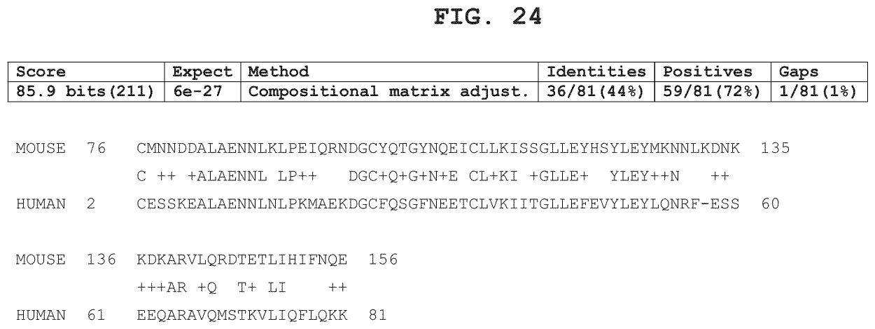

FIG. 23 shows the alignment between human IL6 amino acid sequence (NP_000591.1; SEQ ID NO: 6) and mouse IL6 amino acid sequence (NP_112445.1; SEQ ID NO: 2). FIG. 24 shows the alignment between mouse IL6 amino acid sequence (NP_001300983.1; SEQ ID NO: 4) and human IL6 amino acid sequence (NP_001305024.1; SEQ ID NO: 8). Thus, the corresponding amino acid residue or region between human and mouse IL6 can also be found in FIG. 23 and FIG. 24.

IL6 genes, proteins, and locus of the other species are also known in the art. For example, the gene ID for IL6 in Rattus norvegicus is 24498, the gene ID for IL6 in Macaca mulatta (Rhesus monkey) is 705819, the gene ID for IL6 in Canis lupus familiaris (dog) is 403985, and the gene ID for IL6 in Felis catus (domestic cat) is 493687. The relevant information for these genes (e.g., intron sequences, exon sequences, amino acid residues of these proteins) can be found, e.g., in NCBI database, which are incorporated herein by reference in the entirety.

The present disclosure provides human or chimeric (e.g., humanized) IL6 nucleotide sequence and/or amino acid sequences. In some embodiments, the entire sequence of mouse signal peptide, exon 1, exon 2, exon 3, exon 4 and/or exon 5, are replaced by the corresponding human sequence.

In some embodiments, a “region” or “portion” of mouse signal peptide, exon 1, exon 2, exon 3, exon 4 and/or exon 5 is replaced by the corresponding human sequence.

In some embodiments, the “region” or “portion” can be at least 50%, 55%, 60%, 65%, 70%, 75%, 80%, 85%, 90%, 95%, or 99% identical to signal peptide, exon 1, exon 2, exon 3, exon 4 and/or exon 5. In some embodiments, a region, a portion, or the entire sequence of mouse signal peptide, exon 1, exon 2, exon 3, exon 4 and/or exon 5 is replaced by a region, a portion, or the entire sequence of human signal peptide, exon 1, exon 2, exon 3, exon 4 and/or exon 5. In some embodiments, a “region” or “portion” of mouse signal peptide, exon 1, exon 2, exon 3, exon 4 and/or exon 5 is deleted.

The present disclosure also provides a chimeric (e.g., humanized) IL6 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL6 mRNA sequence (e.g., SEQ ID NO: 1 or 3), mouse IL6 amino acid sequence (e.g., SEQ ID NO: 2 or 4), or a portion thereof (e.g., exon 1, exon 2, exon 3, exon 4 and/or exon 5); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the sequence are identical to or derived from human IL6 mRNA sequence (e.g., SEQ ID NO: 5 or 7), human IL6 amino acid sequence (e.g., SEQ ID NO: 6 or 8), or a portion thereof (e.g., exon 1, exon 2, exon 3, exon 4 and/or exon 5).

In some embodiments, the sequence encoding full-length amino acid sequence of mouse IL6 (SEQ ID NO: 2 or 4) is replaced. In some embodiments, the sequence is replaced by a sequence encoding a corresponding region of human IL6 (e.g., full-length amino acid sequence of human IL6 (SEQ ID NO: 6 or 8)).

In some embodiments, the nucleic acids as described herein are operably linked to a promotor or regulatory element, e.g., an endogenous mouse IL6 promotor, a human IL6 promotor, an inducible promoter, a human enhancer, a mouse enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, or 60 nucleotides, e.g., contiguous or non-contiguous nucleotides) that are different from a portion of or the entire mouse IL6 nucleotide sequence (e.g., exon 1, exon 2, exon 3, exon 4, exon 5, or SEQ ID NO: 1 or 3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, or 60 nucleotides, e.g., contiguous or non-contiguous nucleotides) that is the same as a portion of or the entire mouse IL6 nucleotide sequence (e.g., exon 1, exon 2, exon 3, exon 4, exon 5, or SEQ ID NO: 1 or 3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire human IL6 nucleotide sequence (e.g., exon 1, exon 2, exon 3, exon 4, exon 5, or SEQ ID NO: 5 or 7).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL6 nucleotide sequence (e.g., exon 1, exon 2, exon 3, exon 4, exon 5, or SEQ ID NO: 5 or 7).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse IL6 amino acid sequence (e.g., exon 1, exon 2, exon 3, exon 4, exon 5, or SEQ ID NO: 2 or 4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL6 amino acid sequence (e.g., exon 1, exon 2, exon 3, exon 4, exon 5, or SEQ ID NO: 2 or 4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human IL6 amino acid sequence (e.g., exon 1, exon 2, exon 3, exon 4, exon 5, or SEQ ID NO: 6 or 8).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL6 amino acid sequence (e.g., exon 1, exon 2, exon 3, exon 4, exon 5, or SEQ ID NO: 6 or 8).

In some embodiments, the percentage identity with the sequence shown in SEQ ID NO: 2, 4, 6, or 8 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

Cells, tissues, and animals (e.g., mouse) are also provided that comprise the nucleotide sequences as described herein, as well as cells, tissues, and animals (e.g., mouse) that express human or chimeric (e.g., humanized) IL6 from an endogenous non-human IL6 locus.

In one aspect, the disclosure provides a genetically-modified, non-human animal whose genome comprises at least one chromosome comprising a sequence encoding a human or chimeric IL6.

In some embodiments, the sequence encoding the human or chimeric IL6 is operably linked to an endogenous regulatory element, or a human regulatory element at the endogenous IL6 gene locus in the at least one chromosome.

In some embodiments, the sequence encoding a human or chimeric IL6 comprises a sequence encoding an amino acid sequence that is at least 50%, 55%, 65%, 70%, 75%, 80%, 85%, 90%, 95%, 99%, or 100% identical to human IL6 (SEQ ID NO: 6 or 8).

In some embodiments, the animal is a mammal, e.g., a monkey, a rodent or a mouse. In some embodiments, the animal is a BALB/c mouse or a C57BL/6 mouse.

In some embodiments, the animal does not express endogenous IL6. In some embodiments, the animal has one or more cells expressing human or chimeric IL6.

In some embodiments, the animal has one or more cells expressing human or chimeric IL6, and the expressed human or chimeric IL6 can bind to endogenous IL6R. In some embodiments, the animal has one or more cells expressing human or chimeric IL6, and the expressed human or chimeric IL6 cannot bind to endogenous IL6R.

In another aspect, the disclosure is related to a genetically-modified, non-human animal, wherein the genome of the animal comprises a replacement of a sequence encoding a region of endogenous IL6 with a sequence encoding a corresponding region of human IL6 at an endogenous IL6 gene locus.

In some embodiments, the sequence encoding the corresponding region of human IL6 is operably linked to an endogenous regulatory element, or a human regulatory element at the endogenous IL6 locus, and one or more cells of the animal expresses a chimeric IL6.

In some embodiments, the animal is a mouse, and the replaced endogenous IL6 locus is exon 1, exon 2, exon 3, exon 4 and/or exon 5 of the endogenous mouse IL6 gene.

In some embodiments, the animal is heterozygous with respect to the replacement at the endogenous IL6 gene locus. In some embodiments, the animal is homozygous with respect to the replacement at the endogenous IL6 gene locus.

In another aspect, the disclosure is related to methods for making a genetically-modified, non-human animal. The methods involve replacing in at least one cell of the animal, at an endogenous IL6 gene locus, a sequence encoding a region of an endogenous IL6 with a sequence encoding a corresponding region of human IL6.

In some embodiments, the sequence encoding the corresponding region of human IL6 comprises exon 1, exon 2, exon 3, exon 4 and/or exon 5 of a human IL6 gene.

In some embodiments, the sequence encoding the corresponding region of IL6 comprises at least 50, 75, 100, 125, 150, 175, or 200 nucleotides of exon 1, exon 2, exon 3, exon 4, and/or exon 5 of a human IL6 gene.

In some embodiments, the sequence encoding the corresponding region of human IL6 encodes a sequence that is at least 90% identical to full-length amino acid sequence of SEQ ID NO: 6 or 8.

In some embodiments, the animal is a mouse, and the locus is exon 1, exon 2, exon 3, exon 4, and/or exon 5 of the mouse IL6 gene.

In another aspect, the disclosure is also related to a non-human animal comprising at least one cell comprising a nucleotide sequence encoding a chimeric IL6 polypeptide, wherein the chimeric IL6 polypeptide comprises at least 50 contiguous amino acid residues that are identical to the corresponding contiguous amino acid sequence of a human IL6, wherein the animal expresses the chimeric IL6.

In some embodiments, the chimeric IL6 polypeptide comprises a sequence that is at least 90%, 95%, or 99% identical to full-length amino acid sequence of SEQ ID NO: 6 or 8.

In some embodiments, the nucleotide sequence is operably linked to an endogenous IL6 regulatory element of the animal, a human IL6 regulatory element, a mouse 5′-UTR, a mouse 3′-UTR, a human 5′-UTR, or a human 3′-UTR.

In some embodiments, the nucleotide sequence is integrated to an endogenous IL6 gene locus of the animal.

In some embodiments, the chimeric IL6 has at least one mouse IL6 activity and/or at least one human IL6 activity.

In another aspect, the disclosure is also related to methods of making a genetically-modified mouse cell that expresses a chimeric IL6. The methods involve replacing, at an endogenous mouse IL6 gene locus, a nucleotide sequence encoding a region of mouse IL6 with a nucleotide sequence encoding a corresponding region of human IL6, thereby generating a genetically-modified mouse cell that includes a nucleotide sequence that encodes the chimeric IL6, wherein the mouse cell expresses the chimeric IL6.

In some embodiments, the nucleotide sequence encoding the chimeric IL6 is operably linked to an endogenous regulatory region, or a human IL6 regulatory region, e.g., promoter.

In some embodiments, the animal further comprises a sequence encoding an additional human or chimeric protein (e.g., IL6R, Interleukin 33 (IL33), Interleukin 13 (IL13), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (OX40), CD47, or Signal Regulatory Protein alpha (SIRPα)).

In some embodiments, the additional human or chimeric protein is IL6R.

In one aspect, the disclosure also provides methods of determining effectiveness of an IL6 antagonist (e.g., an anti-IL6 antibody) for reducing inflammation. The methods involve administering the IL6 antagonist to the animal described herein, wherein the animal has an inflammation; and determining the inhibitory effects of the IL6 antagonist to the reduction of inflammation.

In one aspect, the disclosure also provides methods of determining effectiveness of an IL6 antagonist (e.g., an anti-IL6 antibody) for treating autoimmune disorder or allergy. The methods involve administering the IL6 antagonist to the animal described herein, wherein the animal has an autoimmune disorder or allergy; and determining the inhibitory effects of the IL6 antagonist to the treatment of autoimmune disorder or allergy.

In one aspect, the disclosure also provides methods of determining effectiveness of an IL6 antagonist (e.g., an anti-IL6 antibody) for treating cancer. The methods involve administering the IL6 antagonist to the animal as described herein, wherein the animal has a tumor; and determining the inhibitory effects of the IL6 antagonist to the tumor.

In some embodiments, the animal further comprises a sequence encoding a human or chimeric IL6R. In some embodiments, the additional therapeutic agent is an anti-IL6R antibody.

In some embodiments the additional therapeutic agent is an anti-PD-1 antibody, an anti-PD-L1 antibody, an anti-CTLA4 antibody, an anti-CD20 antibody, an anti-EGFR antibody, or an anti-CD319 antibody.

In another aspect, the disclosure further provides methods of determining toxicity of an agent (e.g., an IL6 antagonist). The methods involve administering the agent to the animal as described herein; and determining weight change of the animal. In some embodiments, the method further involve performing a blood test (e.g., determining red blood cell count).

In one aspect, the disclosure relates to proteins comprising an amino acid sequence, wherein the amino acid sequence is one of the following:
(a) an amino acid sequence set forth in SEQ ID NO: 2, 4, 6, 8;
(b) an amino acid sequence that is at least 90%, 91%, 92%, 93%, 94%, 95%, 96%, 97%, 98%, or 99% identical to SEQ ID NO: 2, 4, 6, 8;
(c) an amino acid sequence that is different from the amino acid sequence set forth in SEQ ID NO: 2, 4, 6, 8 by no more than 10, 9, 8, 7, 6, 5, 4, 3, 2 or 1 amino acid; and
(d) an amino acid sequence that comprises a substitution, a deletion and/or insertion of one, two, three, four, five or more amino acids to the amino acid sequence set forth in SEQ ID NO: 2, 4, 6, 8.

In some embodiments, provided herein are cells comprising the proteins disclosed herein. In some embodiments, provided herein are animals having the proteins disclosed herein.

In another aspect, the disclosure relates to nucleic acids comprising a nucleotide sequence, wherein the nucleotide sequence is one of the following:
(a) a sequence that encodes the protein as described herein;
(b) SEQ ID NO: 1, 3, 5, 7, 49, 50;
(c) a sequence that is at least 90%, 91%, 92%, 93%, 94%, 95%, 96%, 97%, 98%, or 99% identical to SEQ ID NO: 1, 3, 5, 7, 49, 50;

In some embodiments, provided herein are cells comprising the nucleic acids disclosed herein. In some embodiments, provided herein are animals having the nucleic acids disclosed herein.

In another aspect, the disclosure also provides a genetically-modified, non-human animal whose genome comprise a disruption in the animal's endogenous IL6 gene, wherein the disruption of the endogenous IL6 gene comprises deletion of exon1, exon2, exon 3, exon 4 and/or exon 5 or part thereof of the endogenous IL6 gene.

In some embodiments, the disruption of the endogenous IL6 gene further comprises deletion of one or more exons or part of exons selected from the group consisting of exon 1, exon 2, exon 3, exon 4 and/or exon 5 of the endogenous IL6 gene.

In some embodiments, the disruption of the endogenous IL6 gene further comprises deletion of one or more introns or part of introns selected from the group consisting of intron 1, intron 2, intron 3 and/or intron 4 of the endogenous IL6 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500 or more nucleotides.

In some embodiments, the disruption of the endogenous IL6 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleotides of exon 1, exon 2, exon 3, exon 4 and/or exon 5 (e.g., deletion of the entire exon 1, exon 2, exon 3, exon 4 and exon 5).

### Interleukin 6 Receptor (IL6R)

The IL-6R (or IL6) signaling cassette includes 2 IL-6R chains and downstream signaling molecules. The IL-6R binding chain exist in 2 forms, first is an 80-kDa transmembrane system and a unique trans-signaling system mediated by the 55-kDa soluble IL-6R (sIL-6R) interacting with IL-6 and then with the membrane bound gp130 (CD130). IL-6 binding to membrane-bound IL-6R activates gp130 and constitutes the signaling pathway for both the membrane-bound and sIL-6R systems. Once gp130 is activated, it results in the downstream activation of the Janus kinase (JAK)-STAT3 pathway and the JAK-SHP-2-mitogen-activated protein (MAP) kinase pathway. Subsequent activation of a number of genes involved in inflammation and immunity then occurs. Termination of IL-6 activation is tightly regulated by suppressor of cytokine synthesis-1 and -3 (SOCS1 and SOCS3).

IL-6/IL-6R interactions represent a classic membrane bound receptor system which is limited to cells that express the IL-6R (primarily hepatocytes and immune cells). However, signaling through the sIL-6R contrast greatly with classic IL-6R signaling and is referred to as “trans-signaling.” Here, sIL-6R binds to circulating IL-6 forming (IL-6/sIL-6R), which has a tendency to stabilize circulating IL-6, promoting availability to cells that express gp130. This includes most cells in the body, greatly enhancing the biologic effects of IL-6 and extending its pathologic vista. Serum levels of sIL-6R also rise with inflammation and are often considered an important marker for acute and chronic tissue inflammation. sIL-6R in humans can be generated by 1 of 2 mechanisms. First, splice mutations of the IL-6R can result in sIL-6R. Second, human IL-6R is cleaved by the adamalysin proteases (ADAM17 and ADAM10), probably after an inflammatory stimulus of polymorphonuclear leukocytes. Cleavage is at a site proximal to the plasma membrane. In addition, cleavage of soluble glycoprotein 130 (sgp130) can also occur and result in blockade of the IL-6/sIL-6R complex. This results in the inability of IL-6/sIL-6R to activate cells that express gp130. In essence, sgp130 can be considered a selective inhibitor of the IL-6/sIL-6R trans-signaling pathway.

A detailed description of IL6R and its function can be found, e.g., in Jordan et al. “Interleukin-6, a cytokine critical to mediation of inflammation, autoimmunity and allograft rejection: therapeutic implications of IL-6 receptor blockade.” Transplantation 101.1 (2017): 32-44; Baran, Paul, et al. “The balance of interleukin (IL)-6, IL-6 soluble IL-6 receptor (sIL-6R), and IL-6 sIL-6R sgp130 complexes allows simultaneous classic and trans-signaling.” Journal of Biological Chemistry 293.18 (2018): 6762-6775; each of which is incorporated by reference herein in the entirety.

In human genomes, IL6R gene (Gene ID: 3570) locus has 10 exons, exon 1, exon 2, exon 3, exon 4, exon 5, exon 6, exon 7, exon 8, exon 9, and exon 10. The IL6R protein has an extracellular region, a transmembrane region, and a cytoplasmic region. The nucleotide sequence for human IL6R mRNA is NM_000565.3 (SEQ ID NO: 61), and the amino acid sequence for human IL6R is NP_000556.1 (SEQ ID NO: 62). The location for each exon and each region in human IL6R nucleotide sequence and amino acid sequence is listed below:

TABLE 5

| | NM_000565.3 | NP_000556.1 |
|---|---|---|
| Human IL6R | 5928 bp | 468aa |
| (approximate location) | (SEQ ID NO: 61) | (SEQ ID NO: 62) |
| Exon 1 | 1-522 | 1-28 |
| Exon 2 | 523-771 | 29-111 |
| Exon 3 | 772-895 | 112-153 |
| Exon 4 | 896-1077 | 154-213 |
| Exon 5 | 1078-1244 | 214-269 |
| Exon 6 | 1245-1386 | 270-316 |
| Exon 7 | 1387-1433 | 317-332 |
| Exon 8 | 1434-1503 | 333-355 |
| Exon 9 | 1504-1597 | 356-387 |
| Exon 10 | 1598-5914 | 388-468 |
| Signal peptide | 438-494 | 1-19 |
| Extracellular region | 495-1532 | 20-365 |
| (excluding signal peptide | | |
| region) | | |
| Transmembrane region | 1533-1595 | 366-386 |
| Cytoplasmic region | 1596-1841 | 387-468 |
| Donor region in Example | 438-1844 | 1-468 |

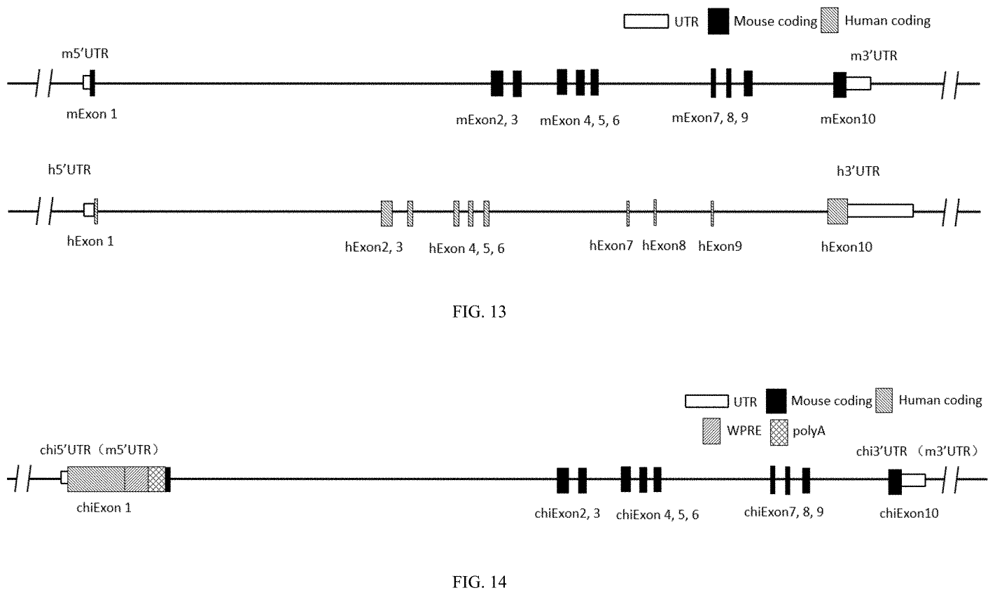

In mice, IL,6R gene locus has 10 exons, exon 1, exon 2, exon 3, exon 4, exon 5, exon 6, exon 7, exon 8, exon 9, and exon 10 (FIG. 13). The mouse IL,6R protein also has an extracellular region, a transmembrane region, and a cytoplasmic region. The nucleotide sequence for mouse IL,6R mRNA is NM_010559.3 (SEQ ID NO: 59), the amino acid sequence for mouse IL,6R is NP_034689.2 (SEQ ID NO: 60). The location for each exon and each region in the mouse TL6R nucleotide sequence and amino acid sequence is listed below:

TABLE 6

| | NM_010559.3 | NP_034689.2 |
|---|---|---|
| Mouse IL6R | 3377 bp | 460aa |
| (approximate location) | (SEQ ID NO: 59) | (SEQ ID NO: 60) |
| Exon 1 | 1-242 | 1-28 |
| Exon 2 | 243-479 | 29-107 |
| Exon 3 | 480-603 | 108-149 |
| Exon 4 | 604-788 | 150-210 |
| Exon 5 | 789-955 | 211-266 |
| Exon 6 | 956-1097 | 267-313 |
| Exon 7 | 1098-1144 | 314-329 |
| Exon 8 | 1145-1214 | 330-352 |
| Exon 9 | 1215-1314 | 353-386 |
| Exon 10 | 1315-3377 | 387-460 |
| Signal peptide | 158-214 | 1-19 |
| Extracellular region | 215-1249 | 20-364 |
| (excluding signal peptide | | |
| region) | | |
| Transmembrane region | 1250-1312 | 365-385 |
| Cytoplasmic region | 1313-1537 | 386-460 |

The mouse IL6R gene (Gene ID: 16194) is located in Chromosome 3 of the mouse genome, which is located from 89869324 to 89913196, of NC_000069.6 (GRCm38.p4 (GCF_000001635.24)).

The 5′-UTR is from 89,913,162 to 89,913,040, exon 1 is from 89,913,162 to 89,912,955, the first intron is from 89,912,954 to 89,890,474, exon 2 is from 89,890,473 to 89,890,237, the second intron is from 89,890,236 to 89,889,311, exon 3 is from 89,889,310 to 89,889,187, the third intron is from 89,889,186 to 89,887,207, exon 4 is from 89,887,206 to 89,887,022, the fourth intron is from 89,887,021 to 89,886,709, exon 5 is from 89,886,708 to 89,886,542, the fifth intron is from 89,886,541 to 89,886,044, the exon 6 is from 89,886,043 to 89,885,902, the sixth intron is from 89,885,901 to 89,878,896, the exon 7 is from 89,878,895 to 89,878,849, the seventh intron is from 89,878,848 to 89,877,914, the exon 8 is from 89,877,913 to 89,877,844, the eighth intron is from 89,877,843 to 89,876,909, the exon 9 is from 89,876,908 to 89,876,809, the ninth intron is from 89,876,808 to 89,871,387, the exon 10 is from 89,871,386 to 89,869,324, the 3′-UTR is from 89,871,160 to 89,869,324, based on transcript NM_010559.3. All relevant information for mouse IL6R locus can be found in the NCBI website with Gene ID: 16194, which is incorporated by reference herein in its entirety.

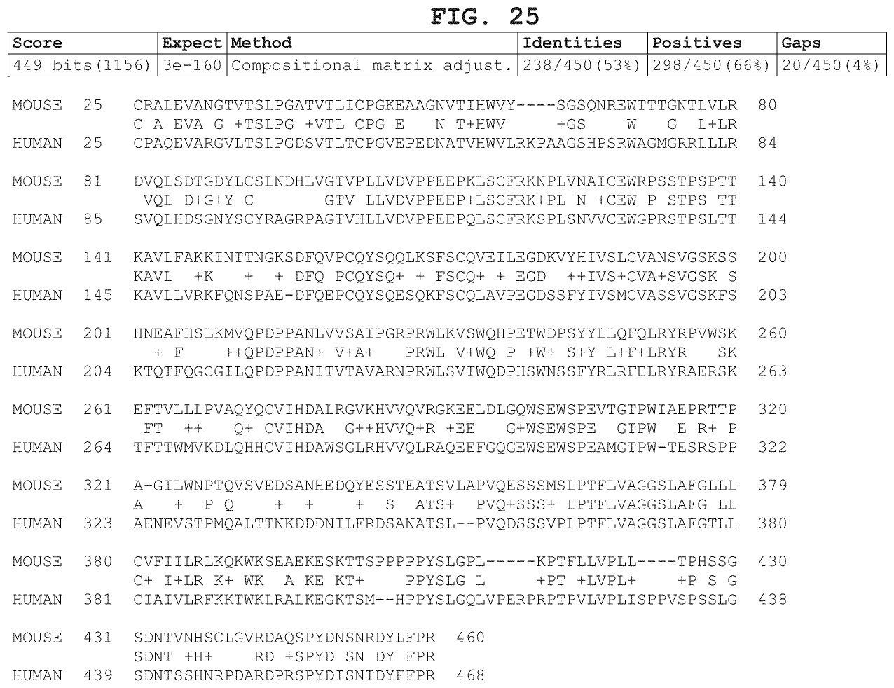

FIG. 25 shows the alignment between human IL6R amino acid sequence (NP_000556.1; SEQ ID NO: 62) and mouse TL6R amino acid sequence (NP_034689.2; SEQ ID NO: 60). Thus, the corresponding amino acid residue or region between human and mouse IL6R can also be found in FIG. 25.

IL6R genes, proteins, and locus of the other species are also known in the art. For example, the gene ID for IL6R in Rattus norvegicus is 24499, the gene ID for IL6R in Macaca mulatta (Rhesus monkey) is 716690, the gene ID for IL6R in Canis lupus familiaris (dog) is 612271, and the gene ID for IL6R in Sus scrofa (pig) is 399522. The relevant information for these genes (e.g., intron sequences, exon sequences, amino acid residues of these proteins) can be found, e.g., in NCBI database.

The present disclosure provides human or chimeric (e.g., humanized) IL6R nucleotide sequence and/or amino acid sequences. In some embodiments, the entire sequence of mouse exon 1, exon 2, exon 3, exon 4, exon 5, exon 6, exon 7, exon 8, exon 9, exon 10, the signal peptide, the extracellular region, the transmembrane region, and/or the cytoplasmic region are replaced by the corresponding human sequence.

In some embodiments, a “region” or “portion” of mouse exon 1, exon 2, exon 3, exon 4, exon 5, exon 6, exon 7, exon 8, exon 9, exon 10, signal peptide, the extracellular region, the transmembrane region, and/or the cytoplasmic region is replaced by the corresponding human sequence. In some embodiments, a “region” or “portion” of human exon 1, exon 2, exon 3, exon 4, exon 5, exon 6, exon 7, exon 8, exon 9, exon 10, signal peptide, the extracellular region, the transmembrane region, the cytoplasmic region, and/or the coding sequence is inserted into the animal genome. The term “region” or “portion” can refer to at least 1, 2, 3, 4, 5, 6, 7, 8, 9, 10, 20, 30, 40, 50, 60, 70, 80, 90, 100, 110, 120, 130, 150, 200, 250, 300, 350, or 400 nucleotides, or at least 1, 2, 3, 4, 5, 6, 7, 8, 9, 10, 20, 30, 40, 50, 60, 70, 80, 90, 100, 110, 120, 130, or 150 amino acid residues.

In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, exon 10, signal peptide, the extracellular region, the transmembrane region, and/or the cytoplasmic region. In some embodiments, a region, a portion, or the entire sequence of mouse exon 1, exon 2, exon 3, exon 4, exon 5, exon 6, exon 7, exon 8, exon 9, and/or exon 10 (e.g., exon 1, exon 2, exon 3, exon 4, exon 5, exon 6, exon 7, exon 8, exon 9 and exon 10) is replaced by a region, a portion, or the entire sequence of human exon 1, exon 2, exon 3, exon 4, exon 5, exon 6, exon 7, exon 8, exon 9, and/or exon 10 (e.g., exon 1, exon 2, exon 3, exon 4, exon 5, exon 6, exon 7, exon 8, exon 9 and exon 10).

In some embodiments, a “region” or “portion” of mouse exon 1, exon 2, exon 3, exon 4, exon 5, exon 6, exon 7, exon 8, exon 9, exon 10, signal peptide, the extracellular region, the transmembrane region, and/or the cytoplasmic region is inactivated or deleted. For example, a region or a portion of exon 1, exon 2, exon 3, exon 4, exon 5, exon 6, exon 7, exon 8, exon 9 and exon 10 is deleted.

Thus, in some embodiments, the present disclosure also provides a chimeric (e.g., humanized) IL6R nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL6R mRNA sequence (e.g., SEQ ID NO: 59), mouse IL6R amino acid sequence (e.g., SEQ ID NO: 60), or a portion thereof (e.g., exon 1, exon 2, exon 3, exon 4, exon 5, exon 6, exon 7, exon 8, exon 9, and/or exon 10). In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL6R mRNA sequence (e.g., SEQ ID NO: 61), human IL6R amino acid sequence (e.g., SEQ ID NO: 62), or a portion thereof (e.g., exon 1, exon 2, exon 3, exon 4, exon 5, exon 6, exon 7, exon 8, exon 9, and/or exon 10).

In some embodiments, the sequence encoding full-length amino acids of mouse IL6R (SEQ ID NO: 60) is replaced or inactivated. In some embodiments, the sequence is replaced by a sequence encoding a corresponding region of human IL6R (e.g., full-length amino acids of human IL6R (SEQ ID NO: 62).

In some embodiments, the nucleic acids as described herein are operably linked to a promotor or regulatory element, e.g., an endogenous mouse IL6R promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse IL6R nucleotide sequence (e.g., exon 1, exon 2, exon 3, exon 4, exon 5, exon 6, exon 7, exon 8, exon 9, exon 10, or SEQ ID NO: 59).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse IL6R nucleotide sequence (e.g., exon 1, exon 2, exon 3, exon 4, exon 5, exon 6, exon 7, exon 8, exon 9, exon 10, or SEQ ID NO: 59).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire human IL6R nucleotide sequence (e.g., exon 1, exon 2, exon 3, exon 4, exon 5, exon 6, exon 7, exon 8, exon 9, exon 10, or SEQ ID NO: 61).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL6R nucleotide sequence (e.g., exon 1, exon 2, exon 3, exon 4, exon 5, exon 6, exon 7, exon 8, exon 9, exon 10, or SEQ ID NO: 61).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse IL6R amino acid sequence (e.g., exon 1, exon 2, exon 3, exon 4, exon 5, exon 6, exon 7, exon 8, exon 9, exon 10, or SEQ ID NO: 60).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL6R amino acid sequence (e.g., exon 1, exon 2, exon 3, exon 4, exon 5, exon 6, exon 7, exon 8, exon 9, exon 10, or SEQ ID NO: 60).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human IL6R amino acid sequence (e.g., exon 1, exon 2, exon 3, exon 4, exon 5, exon 6, exon 7, exon 8, exon 9, exon 10, or SEQ ID NO: 62).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL6R amino acid sequence (e.g., exon 1, exon 2, exon 3, exon 4, exon 5, exon 6, exon 7, exon 8, exon 9, exon 10, or SEQ ID NO: 62).

The present disclosure also provides a humanized TL6R mouse amino acid sequence, wherein the amino acid sequence is selected from the group consisting of:

a) an amino acid sequence shown in SEQ ID NO: 60 or 62;

b) an amino acid sequence having a homology of at least 90% with or at least 90% identical to the amino acid sequence shown in SEQ ID NO: 60 or 62;

c) an amino acid sequence encoded by a nucleic acid sequence, wherein the nucleic acid sequence is able to hybridize to a nucleotide sequence encoding the amino acid shown in SEQ ID NO: 60 or 62 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 60 or 62;

e) an amino acid sequence that is different from the amino acid sequence shown in SEQ ID NO: 60 or 62 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 60 or 62.

The present disclosure also relates to a nucleic acid (e.g., DNA or RNA) sequence, wherein the nucleic acid sequence can be selected from the group consisting of:

a) a nucleic acid sequence as shown in SEQ ID NO: 59, 61 or 65, or a nucleic acid sequence encoding a homologous IL6R amino acid sequence of a humanized mouse;

b) a nucleic acid sequence that is able to hybridize to the nucleotide sequence as shown in SEQ ID NO: 59, 61 or 65 under a low stringency condition or a strict stringency condition;

c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 59, 61 or 65;

d) a nucleic acid sequence that encodes an amino acid sequence, wherein the amino acid sequence has a homology of at least 90% with or at least 90% identical to the amino acid sequence shown in SEQ ID NO: 59, 61 or 65;

e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 59, 61 or 65;

f) a nucleic acid sequence that encodes an amino acid sequence, wherein the amino acid sequence is different from the amino acid sequence shown in SEQ ID NO: 59, 61 or 65 by no more than 10, 9, 8, 7, 6, 5, 4, 3, 2 or no more than 1 amino acid; and/or

g) a nucleic acid sequence that encodes an amino acid sequence, wherein the amino acid sequence comprises a substitution, a deletion and/or insertion of one or more amino acids to the amino acid sequence shown in SEQ ID NO: 59, 61 or 65.

The present disclosure further relates to an IL6R genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 59, 61 or 65.

The disclosure also provides an amino acid sequence that has a homology of at least 90% with, or at least 90% identical to the sequence shown in SEQ ID NO: 60 or 62, and has protein activity. In some embodiments, the homology with the sequence shown in SEQ ID NO: 60 or 62 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 60 or 62 is at least or about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 59, 61 or 65, and encodes a polypeptide that has protein activity. In some embodiments, the homology with the sequence shown in SEQ ID NO: 59, 61 or 65 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

Cells, tissues, and animals (e.g., mouse) are also provided that comprise the nucleotide sequences as described herein, as well as cells, tissues, and animals (e.g., mouse) that express human or chimeric (e.g., humanized) IL6R from an endogenous non-human IL6R locus.

In one aspect, the disclosure also provides methods of determining effectiveness of an IL6R antagonist (e.g., an anti-IL6R antibody) for reducing inflammation. The methods involve administering the IL6R antagonist to the animal described herein, wherein the animal has an inflammation; and determining the inhibitory effects of the IL6R antagonist to the reduction of inflammation.

In one aspect, the disclosure also provides methods of determining effectiveness of an IL6R antagonist (e.g., an anti-IL6R antibody) for treating autoimmune disorder or allergy. The methods involve administering the IL6R antagonist to the animal described herein, wherein the animal has an autoimmune disorder or allergy; and determining the inhibitory effects of the IL6R antagonist to the treatment of autoimmune disorder or allergy.

In one aspect, the disclosure also provides methods of determining effectiveness of an IL6R antagonist (e.g., an anti-IL6R antibody) for treating cancer. The methods involve administering the IL6R antagonist to the animal described herein, wherein the animal has a tumor; and determining the inhibitory effects of the IL6R antagonist to the tumor. In some embodiments, the tumor comprises one or more cancer cells that are injected into the animal. In some embodiments, determining the inhibitory effects of the IL6R antagonist (e.g., an anti-IL6R antibody) to the tumor involves measuring the tumor volume in the animal.

In another aspect, the disclosure also provides a genetically-modified, non-human animal whose genome comprise a disruption in the animal's endogenous IL6R gene, wherein the disruption of the endogenous IL6R gene comprises deletion of exon 1, exon 2, exon 3, exon 4, exon 5, exon 6, exon 7, exon 8, exon 9, exon 10, (e.g., exon 1, exon 2, exon 3, exon 4, exon 5, exon 6, exon 7, exon 8, exon 9 and/or exon 10) or part thereof of the endogenous IL6R gene.

In some embodiments, the disruption of the endogenous IL6R gene comprises deletion of one or more exons or part of exons selected from the group consisting of exon 1, exon 2, exon 3, exon 4, exon 5, exon 6, exon 7, exon 8, exon 9 and/or exon 10 of the endogenous TL6R gene.

In some embodiments, the disruption of the endogenous IL6R gene further comprises deletion of one or more introns or part of introns selected from the group consisting of intron 1, intron 2, intron 3, intron 4, intron 5, intron 6, intron 7, intron 8 and/or intron 9 of the endogenous IL6R gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500 or more nucleotides.

In some embodiments, the disruption of the endogenous IL6R gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleotides of exon 1, exon 2, exon 3, exon 4, exon 5, exon 6, exon 7, exon 8, exon 9, exon 10 (e.g., exon1, exon 2, exon 3, exon 4, exon 5, exon 6, exon 7, exon 8, exon 9 and/or exon 10).

### Genetically Modified Animals

As used herein, the term “genetically-modified non-human animal” refers to a non-human animal having genetic modification (e.g., exogenous DNA) in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the genetic modification in its genome. The cell having exogenous DNA can be various kinds of cells, e.g., an endogenous cell, a somatic cell, an immune cell, a T cell, a B cell, a germ cell, a blastocyst, or an endogenous tumor cell. In some embodiments, genetically-modified non-human animals are provided that comprise a modified endogenous IL6R or IL6 locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one portion of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

In some embodiments, the chimeric gene or the chimeric nucleic acid is a humanized IL6R gene or a humanized IL6R nucleic acid. In some embodiments, at least one or more portions of the gene or the nucleic acid is from the human IL6R gene, at least one or more portions of the gene or the nucleic acid is from a non-human IL6R gene. In some embodiments, the gene or the nucleic acid comprises a sequence that encodes an IL6R protein. The encoded IL6R protein is functional or has at least one activity of the human IL6R protein or the non-human IL6R protein, e.g., binding to human or non-human IL6, and/or upregulating immune response.

In some embodiments, the chimeric protein or the chimeric polypeptide is a humanized IL6R protein or a humanized IL6R polypeptide. In some embodiments, at least one or more portions of the amino acid sequence of the protein or the polypeptide is from a human IL6R protein, and at least one or more portions of the amino acid sequence of the protein or the polypeptide is from a non-human IL6R protein. The humanized IL6R protein or the humanized IL6R polypeptide is functional or has at least one activity of the human IL6R protein or the non-human IL6R protein.

In some embodiments, the humanized IL6R protein or the humanized IL6R polypeptide can bind to mouse IL6, and/or upregulate immune response. In some embodiments, the humanized IL6R protein or the humanized IL6R polypeptide cannot bind to mouse IL6, thus cannot upregulate immune response.

In some embodiments, the chimeric gene or the chimeric nucleic acid is a humanized IL6 gene or a humanized IL6 nucleic acid. In some embodiments, at least one or more portions of the gene or the nucleic acid is from the human IL6 gene, at least one or more portions of the gene or the nucleic acid is from a non-human IL6 gene. In some embodiments, the gene or the nucleic acid comprises a sequence that encodes an IL6 protein. The encoded IL6 protein is functional or has at least one activity of the human IL6 protein or the non-human IL6 protein, e.g., binding to human or non-human IL6R, and/or upregulating immune response.

In some embodiments, the chimeric protein or the chimeric polypeptide is a humanized IL6 protein or a humanized IL6 polypeptide. In some embodiments, at least one or more portions of the amino acid sequence of the protein or the polypeptide is from a human IL6 protein, and at least one or more portions of the amino acid sequence of the protein or the polypeptide is from a non-human IL6 protein. The humanized IL6 protein or the humanized IL6 polypeptide is functional or has at least one activity of the human IL6 protein or the non-human IL6 protein.

In some embodiments, the humanized IL6 protein or the humanized IL6 polypeptide can bind to mouse IL6R, and/or upregulate immune response. In some embodiments, the humanized IL6 protein or the humanized IL6 polypeptide cannot bind to mouse IL6R, thus cannot upregulate immune response.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 12951/SV, 12951/SvIm), 129S2, 129S4, 129S5, 12959/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized IL6R or IL6 animal is made. For example, suitable mice for maintaining a xenograft, can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100(9): 3175-3182, 2002), B-NDG mice (Zhang, Meiling, et al. “The B-NDG mouse is a perfect tool for immune system humanization and patient derived xenograft transplantation.” AACR; Cancer Res 2018; 78(13 Suppl): Abstract nr 1157, or US20190320631, both of which are incorporated herein by reference in its entirety), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human IL6R or IL6 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, B-NDG mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature IL6R or IL6 coding sequence with human mature IL6R or IL6 coding sequence.

In some embodiments, the genetically-modified, non-human animal comprises a disruption in the animal's endogenous CD132 gene, wherein the disruption of the endogenous CD132 gene comprises deletion of exon 2 of the endogenous CD132 gene.

In some embodiments, the disruption of the endogenous CD132 gene further comprises deletion of exon 1 of the endogenous CD132 gene. In some embodiments, the disruption of the endogenous CD132 gene comprises deletion of part of exon 1 of the endogenous CD132 gene. In some embodiments, the disruption of the endogenous CD132 gene further comprises deletion of one or more exons or part of exons selected from the group consisting of exon 3, exon 4, exon 5, exon 6, exon 7, and exon 8 of the endogenous CD132 gene. In some embodiments, the disruption of the endogenous CD132 gene comprises deletion of exons 1-8 of the endogenous CD132 gene. In some embodiments, the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene. In some embodiments, the disruption consists of deletion of more than 150 nucleotides in exon 1; deletion of the entirety of intron 1, exon 2, intron 2, exon 3, intron 3, exon 4, intron 4, exon 5, intron 5, exon 6, intron 6, exon 7, intron 7; and deletion of more than 250 nucleotides in exon 8.

In some embodiments, the animal is homozygous with respect to the disruption of the endogenous CD132 gene. In some embodiments, the animal is heterozygous with respect to the disruption of the endogenous CD132 gene.

In some embodiments, the disruption prevents the expression of functional CD132 protein.

In some embodiments, the length of the remaining exon sequences at the endogenous CD132 gene locus is less than 30% of the total length of all exon sequences of the endogenous CD132 gene. In some embodiments, the length of the remaining sequences at that the endogenous CD132 gene locus is less than 15% of the full sequence of the endogenous CD132 gene.

In some embodiments, the animal is a CD132 knockout non-human animal, wherein the genome of the animal comprises from 5′ to 3′ at the endogenous CD132 gene locus, (a) a first DNA sequence; optionally (b) a second DNA sequence comprising an exogenous sequence; (c) a third DNA sequence, wherein the first DNA sequence, the optional second DNA sequence, and the third DNA sequence are linked, wherein the first DNA sequence comprises an endogenous CD132 gene sequence that is located upstream of intron 1, the second DNA sequence can have a length of 0 nucleotides to 300 nucleotides, and the third DNA sequence comprises an endogenous CD132 gene sequence that is located downstream of intron 7.

In some embodiments, the first DNA sequence comprises a sequence that has a length (5′ to 3′) of from 10 to 100 nucleotides (e.g., approximately 10, 20, 30, 40, 50, 60, 70, 80, 90, 100 nucleotides), wherein the length of the sequence refers to the length from the first nucleotide in exon 1 of the CD132 gene to the last nucleotide of the first DNA sequence.

In some embodiments, the first DNA sequence comprises at least 10 nucleotides from exon 1 of the endogenous CD132 gene. In some embodiments, the first DNA sequence has at most 100 nucleotides from exon 1 of the endogenous CD132 gene.

In some embodiments, the third DNA sequence comprises a sequence that has a length (5 to 3′) of from 200 to 600 nucleotides (e.g., approximately 200, 250, 300, 350, 400, 450, 500, 550, 600 nucleotides), wherein the length of the sequence refers to the length from the first nucleotide in the third DNA sequence to the last nucleotide in exon 8 of the endogenous CD132 gene.

In some embodiments, the third DNA sequence comprises at least 300 nucleotides from exon 8 of the endogenous CD132 gene. In some embodiments, the third DNA sequence has at most 400 nucleotides from exon 8 of the endogenous CD132 gene.

In some embodiments, the animal is a genetically-modified, non-human animal produced by a method comprising knocking out one or more exons of endogenous CD132 gene by using (1) a first nuclease comprising a zinc finger protein, a TAL-effector domain, or a single guide RNA (sgRNA) DNA-binding domain that binds to a target sequence in exon 1 of the endogenous CD132 gene or upstream of exon 1 of the endogenous CD132 gene, and (2) a second nuclease comprising a zinc finger protein, a TAL-effector domain, or a single guide RNA (sgRNA) DNA-binding domain that binds to a sequence in exon 8 of the endogenous CD132 gene.

The animal with a disruption at CD132 gene is described in US20190320631, which is incorporated herein by reference in its entirety.

In some embodiments, the animal is a mammal, e.g., a monkey, a rodent, a rat, or a mouse. In some embodiments, the animal is a NOD mouse, a NOD/scid mouse, or a NOD/scid nude mouse. In some embodiments, the animal further comprises a disruption in the animal's endogenous Beta-2-Microglobulin (B2m) gene and/or a disruption in the animal's endogenous Forkhead Box N1 (Foxn1) gene.

Genetically modified non-human animals can comprise a modification of an endogenous non-human IL6 or IL6R locus. In some embodiments, the modification can comprise a human nucleic acid sequence encoding at least a portion of a mature IL6 or IL6R protein (e.g., at least 10%, 20%, 30%, 40%, 50%, 60%, 70%, 80%, 90%, 95%, 96%, 97%, 98%, or 99% identical to the mature IL6 or IL6R protein sequence). Although genetically modified cells are also provided that can comprise the modifications described herein (e.g., ES cells, somatic cells), in many embodiments, the genetically modified non-human animals comprise the modification of the endogenous IL6 or IL6R locus in the germline of the animal.

Genetically modified animals can express a human IL6 or IL6R (or a chimeric IL6 or IL6R) from endogenous mouse loci, wherein the endogenous mouse gene has been replaced with a human gene and/or a nucleotide sequence that encodes a region of human IL6 or IL6R sequence or an amino acid sequence that is at least 10%, 20%, 30%, 40%, 50%, 60%, 70&, 80%, 90%, 95%, 96%, 97%, 98%, or 99% identical to the human IL6 or IL6R sequence. In various embodiments, an endogenous non-human locus is modified in whole or in part to comprise human nucleic acid sequence encoding at least one protein-coding sequence of a mature protein.

In some embodiments, the genetically modified mice express the human IL6 or IL6R (or chimeric IL6 or IL6R) from endogenous loci that are under control of mouse promoters, mouse regulatory elements, human promoters, and/or human regulatory elements. The replacement(s) at the endogenous mouse loci provide non-human animals that express human protein or chimeric protein in appropriate cell types and in a manner that does not result in the potential pathologies observed in some other transgenic mice known in the art. The human protein or the chimeric protein expressed in animal can maintain one or more functions of the wild-type mouse or human protein in the animal.

For example, IL6R can bind to human or non-human IL6, and upregulate immune response, e.g., upregulate immune response by at least 10%, 20%, 30%, 40%, or 50%. As used herein, the term “endogenous IL6R” refers to IL6R protein that is expressed from an endogenous IL6R nucleotide sequence of the non-human animal (e.g., mouse) before any genetic modification. Similarly, the term “endogenous IL6” refers to IL6 protein that is expressed from an endogenous IL6 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to SEQ ID NO: 2, 4, 6, or 8, and/or a sequence encoding an amino acid sequence that is at least 70%, 75%, 80%, 85%, 90%, 95%, 99%, or 100% identical to SEQ ID NO: 60 or 62.

The genome of the genetically modified animal can comprise an insertion at an endogenous IL6R gene locus a sequence encoding a region of human IL6R. In some embodiments, the sequence that is inserted comprises one or more sequences selected from, e.g., exon 1, exon 2, exon 3, exon 4, exon 5, exon 6, exon 7, exon 8, exon 9, exon 10, 5′-UTR, 3′UTR, the first intron, the second intron, and the third intron, the fourth intron, the fifth intron, the sixth intron, the seventh intron, the eighth intron, the ninth intron, or the tenth intron, etc. In some embodiments, the sequence that is inserted is within the regulatory region of the endogenous IL6R gene. In some embodiments, the sequence that is inserted is exon 1, exon 2, exon 3, exon 4, exon 5, exon 6, exon 7, exon 8, exon 9 and/or exon 10 or part thereof, of a human IL6R gene.

The genetically modified animal can have one or more cells expressing a human or chimeric IL6R (e.g., humanized IL6R) having an extracellular region and a cytoplasmic region, wherein the extracellular region comprises a sequence that is at least 50%, 60%, 70%, 80%, 90%, 95%, 99% identical to the extracellular region of human IL6R. In some embodiments, the extracellular region of the humanized IL6R has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to human IL6R.

The genome of the genetically modified animal can comprise a replacement at an endogenous IL6 gene locus of a sequence encoding a region of endogenous IL6 with a sequence encoding a corresponding region of human IL6. In some embodiments, the sequence that is replaced is any sequence within the endogenous IL6 gene locus, e.g., exon 1, exon 2, exon 3, exon 4, exon 5, 5′-UTR, 3′UTR, the first intron, the second intron, the third intron, or the fourth intron, etc. In some embodiments, the sequence that is replaced is within the regulatory region of the endogenous IL6 gene. In some embodiments, the sequence that is replaced is within the regulatory region of the human IL6 gene.

Because human protein and non-human protein sequences, in many cases, are different, antibodies that bind to human protein will not necessarily have the same binding affinity with non-human protein or have the same effects to non-human protein. Therefore, the genetically modified animal expressing human IL6 and the genetically modified animal having a human or a humanized extracellular region of IL6R can be used to better evaluate the effects of anti-IL6 or IL6R antibodies in an animal model.

In some embodiments, the non-human animal can have, at an endogenous IL6R gene locus, a nucleotide sequence encoding a chimeric human/non-human IL6R polypeptide, wherein a human portion of the chimeric human/non-human IL6R polypeptide comprises a portion of human IL6R extracellular region, and wherein the animal expresses a functional IL6R on a surface of a cell of the animal. The human portion of the chimeric human/non-human IL6R polypeptide can comprise a portion of exon 1, exon 2, exon 3, exon 4, exon 5, exon 6, exon 7, exon 8, exon 9 and/or exon 10 of human IL6R. In some embodiments, the human portion of the chimeric human/non-human IL6R polypeptide can comprise a sequence that is at least 80%, 85%, 90%, 95%, or 99% identical to SEQ ID NO: 62.

In some embodiments, the humanized TL6R locus lacks a human IL6R 5′-UTR. In some embodiment, the humanized IL6R locus comprises a rodent (e.g., mouse) 5′-UTR. In some embodiments, the humanization comprises a human 3′-UTR. In appropriate cases, it may be reasonable to presume that the mouse and human IL6R genes appear to be similarly regulated based on the similarity of their 5′-flanking sequence. As shown in the present disclosure, humanized IL6R mice that comprise an insertion at an endogenous mouse IL6R locus, which retain mouse regulatory elements but comprise a humanization of IL6R encoding sequence, do not exhibit obvious pathologies. Both genetically modified mice that are heterozygous or homozygous for humanized IL6R are grossly normal.

In some embodiments, the humanized IL6 locus has a human IL6 5′-UTR or an endogenous IL6 5′-UTR. In some embodiment, the humanized IL6 locus comprises a rodent (e.g., mouse) 5′-UTR. In some embodiments, the humanization comprises a human 3′-UTR or an endogenous 3′-URT. In appropriate cases, it may be reasonable to presume that the mouse and human IL6 genes appear to be similarly regulated based on the similarity of their 5′-flanking sequence. As shown in the present disclosure, humanized IL6 mice that comprise a replacement at an endogenous mouse IL6 locus, which has mouse or human regulatory elements, do not exhibit obvious pathologies. Both genetically modified mice that are heterozygous or homozygous for humanized IL6 are grossly normal.

The present disclosure further relates to a non-human mammal generated through the method mentioned above. In some embodiments, the genome thereof contains human gene(s). In some embodiments, the non-human mammal is a rodent, and preferably, the non-human mammal is a mouse.

In some embodiments, the non-human mammal expresses a protein encoded by a humanized IL6R or IL6 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized IL6R or IL6 in the genome of the animal.

In some embodiments, the non-human mammal comprises the genetic construct as described herein. In some embodiments, a non-human mammal expressing human or humanized IL6R or IL6 is provided. In some embodiments, the tissue-specific expression of human or humanized IL6R or IL6 protein is provided.

In some embodiments, the expression of human or humanized IL6R or IL6 in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human IL6R or IL6 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL6R or IL6 protein.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 1, 1%, 20%, 25%, 30%, 35%, 40%4, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) amino acid sequence from an endogenous non-human IL6R or IL6 locus.

### Vectors

The present disclosure relates to a targeting vector, comprising: a) a DNA fragment homologous to the 5′ end of a region to be altered (5′ arm), which is selected from the IL6R or IL6 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL6R or IL6 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000071.6; c) the DNA fragment homologous to the 3′ end of the region to be altered (3′ arm) is selected from the nucleotide sequences that have at least 90% homology to the NCBI accession number NC_000071.6.

In some embodiments, a) the DNA fragment homologous to the 5′ end of a region to be altered (5′ arm) is selected from the nucleotides from the position 30006059 to the position 30011541 of the NCBI accession number NC_000071.6 (SEQ ID NO: 9); c) the DNA fragment homologous to the 3′ end of the region to be altered (3′ arm) is selected from the nucleotides from the position 30020010 to the position 30024779 of the NCBI accession number NC_000071.6 (SEQ ID NO: 10).

In some embodiments, a) the DNA fragment homologous to the 5′ end of a region to be altered (5′ arm) is selected from the nucleotides from the position 30011619 to the position 30013191 of the NCBI accession number NC_000071.6 (SEQ ID NO: 46); c) the DNA fragment homologous to the 3′ end of the region to be altered (3′ arm) is selected from the nucleotides from the position 30019976 to the position 30021303 of the NCBI accession number NC_000071.6 (SEQ ID NO: 47).

In some embodiments, a) the DNA fragment homologous to the 5′ end of a conversion region to be altered (5′ arm) is selected from the nucleotide sequences that have at least 90% homology to the NCBI accession number NC_000069.6; c) the DNA fragment homologous to the 3′ end of the region to be altered (3′ arm) is selected from the nucleotide sequences that have at least 90% homology to the NCBI accession number NC_000069.6.

In some embodiments, a) the DNA fragment homologous to the 5′ end of a region to be altered (5′ arm) is selected from the nucleotides from the position 89917172 to the position 89913040 of the NCBI accession number NC_000069.6 (SEQ ID NO: 63); c) the DNA fragment homologous to the 3′ end of the region to be altered (3′ arm) is selected from the nucleotides from the position 89913026 to the position 89908300 of the NCBI accession number NC_000069.6 (SEQ ID NO: 64).

In some embodiments, the length of the selected genomic nucleotide sequence in the targeting vector can be about or at least 1 kb, 2 kb, 3 kb, 4 kb, 5 kb, 6 kb, 7 kb, 8 kb, 9 kb or 10 kb (e.g., 4.7 kb or 12.7 kb).

In some embodiments, the region to be altered is exon 1, exon 2, exon 3, exon 4, and/or exon 5 of IL6 gene (e.g., exon 1, exon 2, exon 3, exon 4, and/or exon 5 of mouse IL6 gene).

In some embodiments, the region to be altered is exon 1, exon 2, exon 3, exon 4, exon 5, exon 6, exon 7, exon 8, exon 9, and/or exon 10 of IL6R gene (e.g., exon 1, exon 2, exon 3, exon 4, exon 5, exon 6, exon 7, exon 8, exon 9 and/or exon 10 of mouse IL6R gene).

The targeting vector can further include a selected gene marker.

In some embodiments, the sequence of the 5′ arm is shown in SEQ ID NO: 9; and the sequence of the 3′ arm is shown in SEQ ID NO: 10.

In some embodiments, the sequence of the 5′ arm is shown in SEQ ID NO: 46; and the sequence of the 3′ arm is shown in SEQ ID NO: 47.

In some embodiments, the sequence of the 5′ arm is shown in SEQ ID NO: 63; and the sequence of the 3′ arm is shown in SEQ ID NO: 64.

In some embodiments, the sequence is derived from human (e.g., 22722839-22735564 of NC_000007.14, or 22727263-22732018 of NC_000007.14). For example, the target region in the targeting vector is a part or entirety of the nucleotide sequence of a human IL6, preferably exon 1, exon 2, exon 3, exon 4 and/or exon 5 of the human IL6. In some embodiments, the nucleotide sequence of the humanized IL6 encodes the entire or the part of human IL6 protein (e.g., SEQ ID NO: 6 or 8).

In some embodiments, the sequence is derived from human (e.g., 438-1844 of NM_000565.3). For example, the target region in the targeting vector is a part or entirety of the nucleotide sequence of a human IL6R, preferably exon 1, exon 2, exon 3, exon 4, exon 5, exon 6, exon 7, exon 8, exon 9 and/or exon 10 of the human IL6R. In some embodiments, the nucleotide sequence of the humanized IL6R encodes the entire or the part of human IL6R protein (e.g., SEQ ID NO: 62).

In some embodiments, the target region is derived from human. In some embodiments, the target region is a part or entirety of the nucleotide sequence of a humanized IL6R. In some embodiments, the nucleotide sequence is shown as one or more of exon 1, exon 2, exon 3, exon 4, exon 5, exon 6, exon 7, exon 8, exon 9, and/or exon 10 of the human IL6R. In some embodiments, the target region is a part or entirety of the nucleotide sequence of a humanized IL6. In some embodiments, the nucleotide sequence is shown as one or more of exon 1, exon 2, exon 3, exon 4, and/or exon 5 of the human IL6.

In some embodiments, the nucleotide sequence of the human IL6R encodes the human IL6R protein with the NCBI accession number NP_000556.1 (SEQ ID NO: 62). In some emboldens, the nucleotide sequence of the human IL6R is selected from the nucleotides from the position 438 to the position 1844 of NM_000565.3 (1-1407 bp of SEQ ID NO: 65).

In some embodiments, the nucleotide sequence of the human IL6 encodes the human IL6 protein with the NCBI accession number NP_000591.1 (SEQ ID NO: 6) or NP_001305024.1 (SEQ ID NO: 8). In some emboldens, the nucleotide sequence of the human IL6 is selected from the nucleotides from the position 22722839 to the position 22735564 of NC_000007.14 (SEQ ID NO: 11), or position 22727263 to the position 22732018 of NC_000007.14 (SEQ ID NO: 48).

The disclosure also relates to a cell comprising the targeting vectors as described herein.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

In some embodiments, the nucleic acids as described herein are operably linked to a Woodchuck Hepatitis Virus (WHP) Posttranscriptional Regulatory Element (WPRE) and/or a polyA (polyadenylation) signal sequence. The WPRE element is a DNA sequence that, when transcribed, creates a tertiary structure enhancing expression. The sequence can be used to increase expression of genes delivered by viral vectors. WPRE is a tripartite regulatory element with gamma, alpha, and beta components. In some embodiments, the WPRE is at least 80%, 85%, 90%, 95%, 96%, 97%, 98%, 99%, or 100% identical to SEQ ID NO: 87.

The disclosure also provides vectors for constructing a humanized animal model or a knock-out model. In some embodiments, the vectors comprise sgRNA sequence, wherein the sgRNA sequence target IL6 or IL6R gene, and the sgRNA is unique on the target sequence of the gene to be altered, and meets the sequence arrangement rule of 5′-NNN (20)-NGG3′ or 5′-CCN-N(20)-3′; and in some embodiments, the targeting site of the sgRNA in the mouse IL6 gene is located on the exon 1, exon 2, exon 3, exon 4, exon 5, intron 1, intron 2, intron 3, intron 4, upstream of exon 1, or downstream of exon 5 of the mouse IL6 gene.

In some embodiments, the 5′ targeting sequence for the sequence is shown as SEQ ID NOS: 22-28, and the sgRNA sequence recognizes the 5′ targeting site. In some embodiments, the 3′ targeting sequence for the knockout sequence is shown as SEQ ID NOS: 29-36 and the sgRNA sequence recognizes the 3′ targeting site. Thus, the disclosure provides sgRNA sequences for constructing a genetic modified animal model. In some embodiments, the oligonucleotide sgRNA sequences are set forth in SEQ ID NOS: 38-45.

In some embodiments, the disclosure relates to a plasmid construct (e.g., pT7-sgRNA) including the sgRNA sequence, and/or a cell including the construct.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL6R or IL6 gene locus, a sequence encoding a region of an endogenous IL6R or IL6 with a sequence encoding a corresponding region of human or chimeric IL6R or IL6. In some embodiments, the replacement occurs in a germ cell, a somatic cell, a blastocyst, or a fibroblast, etc. The nucleus of a somatic cell or the fibroblast can be inserted into an enucleated oocyte.

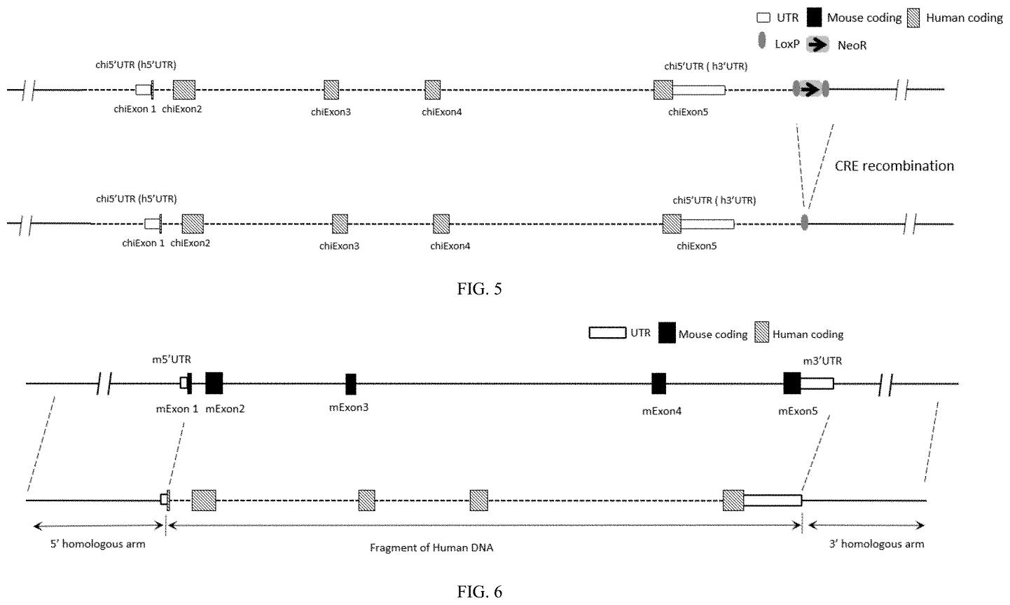

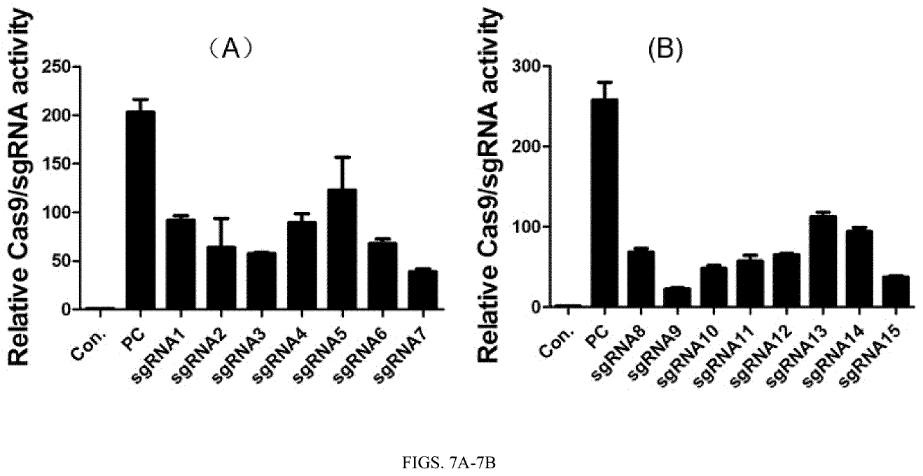

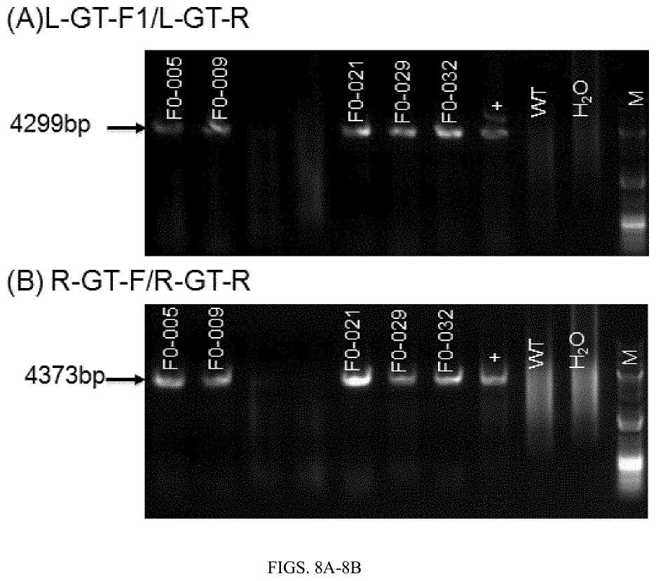

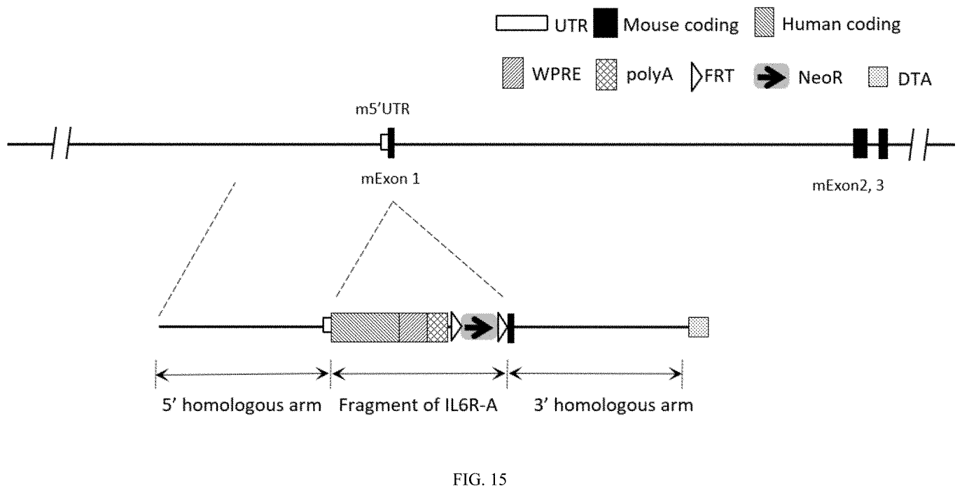

FIG. 15 shows a humanization strategy for a mouse IL6R locus. In FIG. 15, the targeting strategy involves a vector comprising the 5′ end homologous arm, human IL6R gene fragment, WPRE, polyA, Neo cassette, and 3′ homologous arm. The process can involve replacing endogenous IL6R sequence with human sequence by homologous recombination. FIG. 4 and FIG. 6 show a humanization strategy for a mouse IL6 locus. In FIG. 4 and FIG. 6, the targeting strategy involves a vector comprising the 5′ end homologous arm, human IL6 gene fragment, optionally Neo cassette, 3′ homologous arm. The process can involve replacing endogenous IL6 sequence with human sequence by homologous recombination. In some embodiments, the cleavage at the upstream and the downstream of the target site (e.g., by zinc finger nucleases, TALEN or CRISPR) can result in DNA double strand break, and the homologous recombination is used to replace endogenous IL6R or IL6 sequence with human IL6R or IL6 sequence.

Thus, in some embodiments, the methods for making a genetically modified, humanized animal, can include the step of inserting at an endogenous IL6R locus (or site), a nucleic acid encoding a sequence encoding a region of human IL6R. The sequence can include a region (e.g., a part or the entire region) of exon 1, exon 2, exon 3, exon 4, exon 5, exon 6, exon 7, exon 8, exon 9, and/or exon 10 of a human IL6R gene. In some embodiments, the sequence includes a region of exon 1, exon 2, exon 3, exon 4, exon 5, exon 6, exon 7, exon 8, exon 9 and/or exon 10 of a human IL6R gene (e.g., SEQ ID NO: 62). In some embodiments, the inserted site is located within exon 1. In some embodiments, the sequence is inserted immediately after the start codon.

In some embodiments, the methods for making a genetically modified, humanized animal, can include the step of replacing at an endogenous IL6 locus (or site), a nucleic acid encoding a sequence encoding a region of endogenous IL6 with a sequence encoding a corresponding region of human IL6. The sequence can include a region (e.g., a part or the entire region) of exon 1, exon 2, exon 3, exon 4, and/or exon 5 of a human IL6 gene. In some embodiments, the sequence includes a region of exon 1, exon 2, exon 3, exon 4, and/or exon 5 of a human IL6 gene (e.g., SEQ ID NO: 6 or 8). In some embodiments, the endogenous IL6 locus is exon 1, exon 2, exon 3, exon 4, and/or exon 5 of mouse TL6.

In some embodiments, the methods of modifying an IL6R or IL6 locus of a mouse to express a chimeric human/mouse IL6R or IL6 peptide can include the steps of replacing at the endogenous mouse IL6R or IL6 locus a nucleotide sequence encoding a mouse IL6R or IL6 with a nucleotide sequence encoding a human IL6R or IL6, thereby generating a sequence encoding a chimeric human/mouse IL6R or IL6.

In some embodiments, the nucleotide sequence encoding the chimeric human/mouse IL6R can include a first nucleotide sequence comprising a mouse 5′-UTR; a second nucleotide sequence encoding human IL6R; a third nucleotide sequence encoding the mouse IL6R.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing an IL6R or IL6 gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 or BALB/c mouse).

In some embodiments, the non-human mammal in step (c) is a female with pseudo pregnancy (or false pregnancy).

In some embodiments, the fertilized eggs for the methods described above are C57BL/6 or BALB/c fertilized eggs. Other fertilized eggs that can also be used in the methods as described herein include, but are not limited to, FVB/N fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the method described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized IL6R and/or IL6 protein, e.g., in a physiologically appropriate manner, provide a variety of uses that include, but are not limited to, developing therapeutics for human diseases and disorders, and assessing the toxicity and/or efficacy of these human therapeutics in the animal models.

In various aspects, genetically modified animals are provided that express human or humanized IL6R and/or IL6, which are useful for testing agents that can decrease or block the interaction between IL6R and IL6 or the interaction between IL6R and other IL6R ligands, testing whether an agent can increase or decrease the immune response, and/or determining whether an agent is an IL6R or IL6 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In one aspect, the disclosure relates to a method of determining effectiveness of an IL6-IL6R pathway modulator for treating a disease (e.g., reducing inflammation, treating an immune disorder, treating cancer). The method involves administering the IL6-IL6R pathway modulator to the animal as described herein; and determining the effects of the IL6-IL6R pathway modulator on the IL6-IL6R pathway activity.

In one aspect, the disclosure also provides methods of determining effectiveness of an IL6R or IL6 antagonist (e.g., an anti-IL6R or an anti-IL6 antibody) for reducing inflammation. The methods involve administering the IL6R or IL6 antagonist to the animal described herein, wherein the animal has an inflammation; and determining the inhibitory effects of the IL6R or IL6 antagonist to the reduction of inflammation.

In one aspect, the disclosure also provides methods of determining effectiveness of an IL6R or IL6 antagonist (e.g., an anti-IL6R or anti-IL6 antibody) for treating an immune disorder (e.g., an autoimmune disorder or allergy). The methods involve administering the IL6R or IL6 antagonist to the animal described herein, wherein the animal has an immune disorder; and determining the inhibitory effects of the IL6R or IL6 antagonist.

In one aspect, the disclosure also provides methods of determining effectiveness of an IL6R or IL6 antagonist (e.g., an anti-IL6R or anti-IL6 antibody) for treating cancer. The methods involve administering the IL6R or IL6 antagonist to the animal described herein, wherein the animal has a tumor; and determining the inhibitory effects of the IL6R or IL6 antagonist to the tumor. In some embodiments, the tumor comprises one or more cancer cells that are injected into the animal. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In In some embodiments, the anti-IL6R antibody or anti-IL6 antibody prevents IL6 from binding to IL6R. In some embodiments, the anti-IL6R antibody or anti-IL6 antibody cannot prevent IL6 from binding to IL6R (e.g., endogenous IL6R).

In some embodiments, the genetically modified animals can be used for determining whether an anti-IL6R antibody is an IL6R agonist or antagonist. In some embodiments, the genetically modified animals can be used for determining whether an anti-IL6 antibody is an IL6 agonist or antagonist. In some embodiments, the methods as described herein are also designed to determine the effects of the agent (e.g., anti-IL6R or anti-IL6 antibodies) on IL6R and/or IL6, e.g., whether the agent can stimulate macrophages, and/or whether the agent can upregulate the immune response or downregulate immune response. In some embodiments, the genetically modified animals can be used for determining the effective dosage of a therapeutic agent for treating a disease in the subject, e.g., an immune disorder, an allergy, or autoimmune diseases.

In some embodiments, the inhibitory effects are evaluated by paw thickness, an arthritis score, behavioral scores, brain/spinal cord IHC pathology, serum/brain homogenate Th17 type multi-cytokine detection, and/or CNS and spleen flow cytometry. Some of these scores are described, e.g., in Anderson et al. “Rheumatoid arthritis disease activity measures: American College of Rheumatology recommendations for use in clinical practice.” Arthritis care & research 64.5 (2012): 640-647, which is incorporated herein by reference in its entirety.

IL-6 is also one of the major cytokines in the tumour microenvironment. It is known to be deregulated in cancer. Its overexpression has been reported in almost all types of tumors. The strong association between inflammation and cancer is reflected by the high IL-6 levels in the tumor microenvironment, where it promotes tumorigenesis by regulating all hallmarks of cancer and multiple signaling pathways, including apoptosis, survival, proliferation, angiogenesis, invasiveness and metastasis, and, most importantly, the metabolism. Moreover, IL-6 protects the cancer cells from therapy-induced DNA damage, oxidative stress and apoptosis by facilitating the repair and induction of countersignaling (antioxidant and anti-apoptotic/pro-survival) pathways. Therefore, blocking IL-6 or inhibiting its associated signaling independently or in combination with conventional anticancer therapies could be a potential therapeutic strategy for the treatment of cancers with IL-6-dominated signaling (Kumari et al. “Role of interleukin-6 in cancer progression and therapeutic resistance.” Tumor Biology 37.9 (2016): 11553-11572). In some embodiments, the anti-IL6R or anti-IL6 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the antibody is designed for treating various autoimmune diseases or allergy (e.g., allergic rhinitis, sinusitis, asthma, multiple sclerosis or eczema). Thus, the methods as described herein can be used to determine the effectiveness of an antibody in inhibiting immune response.

The present disclosure also provides methods of determining toxicity of an antibody (e.g., anti-IL6R antibody or anti-IL6 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%.

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL6R or IL6 gene function, human IL6R or IL6 antibodies, drugs for human IL6R or IL6 targeting sites, the drugs or efficacies for human IL6R or IL6 targeting sites, the drugs for immune-related diseases and antitumor drugs.

Genetically Modified Animal Model with Multiple Human or Chimeric Genes

The present disclosure further relates to methods for generating genetically modified animal model with multiple human or chimeric genes. The animal can comprise a human or chimeric IL6R gene and a sequence encoding one or more additional human or chimeric protein (e.g., IL6). Alternatively, the animal can comprise a human or chimeric IL6 gene and a sequence encoding one or more additional human or chimeric protein (e.g., IL6R).

In some embodiments, the additional human or chimeric protein can further include e.g., Interleukin 33 (IL33), IL3, Granulocyte-macrophage colony-stimulating factor (GM-CSF), IL13, programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), CD137, TNF Receptor Superfamily Member 4 (TNFRSF4 or OX40), CD47 or SIRPα.

The methods of generating genetically modified animal model with two or more human or chimeric genes (e.g., humanized genes) can include the following steps:

(a) using the methods of introducing human IL6R gene or chimeric IL6R gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric IL6, IL33, IL13, PD-1, CTLA-4, LAG-3, BTLA, PD-L1, CD27, CD28, TIGIT, TIM-3, GITR, OX40, CD137, CD47, or SIRPa. Some of these genetically modified non-human animal are described, e.g., in PCT/CN2017/090320, PCT/CN2017/099577, PCT/CN2017/110435, PCT/CN2017/099576, PCT/CN2017/099574, PCT/CN2017/106024, PCT/CN2017/110494, PCT/CN2017/110435, PCT/CN2017/117984, PCT/CN2018/081628, PCT/CN2017/120388, PCT/CN2017/099575, and PCT/CN2018/081629; each of which is incorporated herein by reference in its entirety.

Similarly, the methods of generating genetically modified animal model can include the following steps:

(a) using the methods of introducing human IL6 gene or chimeric IL6 gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, the humanization is directly performed on a genetically modified animal having a human or chimeric IL6, IL6R, IL33, IL13, PD-1, CTLA-4, BTLA, PD-L1, CD27, CD28, TIGIT, TIM-3, GITR, CD137, OX40, CD47 or SIRPa gene.

In some embodiments, the IL6R humanization is directly performed on a genetically modified animal having a human or chimeric IL6. In some embodiments, the IL6 humanization is directly performed on a genetically modified animal having a human or chimeric IL6R.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-IL6R antibody and an additional therapeutic agent for the treatment. The methods include administering the anti-IL6R antibody and/or the anti-IL6 antibody, and the additional therapeutic agent to the animal, wherein the animal has a tumor; and determining the inhibitory effects of the combined treatment to the tumor. In some embodiments, the additional therapeutic agent is an antibody that specifically binds to IL6, IL6R, IL33, IL13, PD-1, CTLA-4, BTLA, PD-L1, CD27, CD28, TIGIT, TIM-3, GITR, CD137, OX40, CD47 or SIRPa. In some embodiments, the additional therapeutic agent is an anti-CTLA4 antibody (e.g., ipilimumab), an anti-CD20 antibody (e.g., rituximab), an anti-EGFR antibody (e.g., cetuximab), and an anti-CD319 antibody (e.g., elotuzumab), or anti-PD-1 antibody (e.g., nivolumab).

### EXAMPLES

The invention is further described in the following examples, which do not limit the scope of the invention described in the claims.

### Materials and Methods

The following materials were used in the following examples.

ScaI, HindIII, SpeI, BglII, EcoRI, BamHI, SspI and EcoRV restriction enzymes were purchased from NEB with Catalog numbers: R3122M, R3104M, R0133M, R0144M, R3101M, R3136M, R3132M and R3195M, respectively.

C57BL/6 mice were purchased from the China Food and Drugs Research Institute National Rodent Experimental Animal Center.

NOD-Prkdc^{scid}IL-2rg^{null} (B-NDG) mice were obtained from Beijing Biocytogen Co., Ltd. (Catalog Number: B-CM-001).

Cre Tool mice were obtained from Beijing Biocytogen Co., Ltd. (Catalog Number: B-EM-045).

NOD/scid mice were purchased from Beijing HFK Bioscience Co. Ltd.

UCA kit was obtained from Beijing Biocytogen Co., Ltd. (Catalog Number: BCG-DX-001).

Mouse colon cancer cell MC38 was purchased from EK-Bioscience Co., Ltd.

MEGAshortscript™ Kit (Ambion in vitro transcription kit) was purchased from Thermo Fisher Scientific Inc. (Catalog Number: AM1354).

Cas9mRNA was obtained from SIGMA (Catalog Number: CAS9MRNA-1EA).

LEGEND MAX™ Mouse IL-6 ELISA Kit with Pre-coated Plates (mouse IL6 kit) were purchased from BioLegend, Inc. (Catalog Number: 431307).

LEGEND MAX™ Human IL-6 ELISA Kit with Pre-coated Plates (human IL6 kit) were purchased from BioLegend, Inc. (Catalog Number: 430507).

PrimeScript™ 1st strand cDNA Synthesis Kit was purchased from TAKARA Bio USA, Inc. (Catalog Number: 6110A).

RNAprep pure Cell/Bacteria Kit (culture cell/bacterial total RNA extraction kit) was purchased from Tiangen Biotech (Beijing) Co., Ltd. (Catalog Number: DP430).

APC/Cy7 anti-mouse TCR β chain Antibody (mTcRβ-APC/Cy7) was purchased from BioLegend, Inc. (Catalog Number: 109220).

PE anti-mouse CD126 (IL-6Rα chain) Antibody (mIL-6R PE) was purchased from BioLegend, Inc. (Catalog Number: 115805).

PE anti-human CD126 (IL-6Rα) Antibody (hIL-6R PE) was purchased from BioLegend, Inc. (Catalog Number: 352803).

### Example 1: Mice with Humanized IL6 Gene

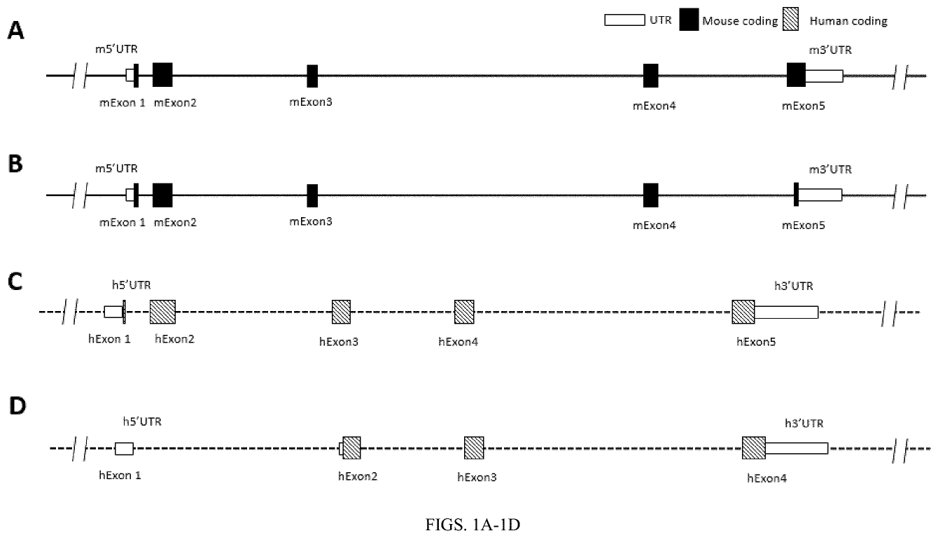

The mouse IL6 gene (NCBI Gene ID: 16193, Primary source: MGI: 96559, UniProt ID: P08505) is located at 30013114 to 30019975 of chromosome 5 (NC_000071.6), and the human IL6 gene (NCBI Gene ID: 3569, Primary source: HGNC: 6018, UniProt ID: P05231) is located at 22725889 to 22732002 of chromosome 7 (NC_000007.14), both having multiple isoforms or transcripts as shown in FIGS. 1A-1D. FIG. 1A shows the mouse transcript NM_031168.2 (SEQ ID NO: 1) and the corresponding protein sequence NP_112445.1 (SEQ ID NO: 2); FIG. 1B shows the mouse transcript NM_001314054.1 (SEQ ID NO: 3) and the corresponding protein sequence NP_001300983.1 (SEQ ID NO: 4); FIG. 1C shows the human transcript NM_000600.4 (SEQ ID NO: 5) and the corresponding protein sequence NP_000591.1 (SEQ ID NO: 6); FIG. 1D shows the human transcript NM_001318095.1 (SEQ ID NO: 7) and the corresponding protein sequence NP_001305024.1 (SEQ ID NO: 8).

For the purpose of the experiments, a gene sequence encoding the human IL6 protein can be introduced into the endogenous mouse IL6 locus, such that the mouse can express a human IL6 protein. Mouse cells can be modified by various gene editing techniques, for example, replacement of specific mouse IL6 gene sequences with human IL6 gene sequences at the endogenous mouse IL6 locus. Under control of a mouse or human IL6 regulatory element, a sequence about 6.2 kb starting from ATG (start codon) to TGA (stop codon) was replaced with a corresponding human DNA sequence to obtain a humanized IL6 locus, thereby humanizing mouse IL6 gene. One of the humanization strategies of the humanized IL6 locus is shown in FIG. 2. The human IL6 coding sequence is under the control of human 5′ UTR.

Another IL6 mouse humanization strategy is to replace a shorter sequence of the IL6 gene, and the humanized mouse IL6 locus is shown in FIG. 3. The human IL6 coding sequence is under the control of mouse 5′-UTR.

In the following experiments, the mouse transcript NM_031168.2→NP_112445.1 and the human transcript NM_000600.4→NP_000591.1 were used as examples.

As shown in the schematic diagram of the targeting strategy in FIG. 4, the recombinant vector contained the homologous arm sequence upstream and downstream of mouse IL6 (about 5.5 kb upstream and about 4.8 kb downstream of endogenous IL6 gene), and 1.2 kb human IL6 Sequence. The upstream homologous arm sequence (5′ homologous arm, SEQ ID NO: 9) was identical to the nucleotide sequence of 30006059-30011541 with NCBI accession number NC_000071.6, and the downstream homologous arm sequence (3′ homologous arm, SEQ ID NO: 10) was identical to the nucleotide sequence of 30020010-30024779 with NCBI accession number NC_000071.6. The DNA fragment sequence of human IL6 (SEQ ID NO: 11) was identical to the nucleotide sequence of 22722839-22735564 with NCBI accession number NC_000007.14.

The targeting vector also included an antibiotic resistance gene for positive clone screening (neomycin phosphotransferase Neo), and two LoxP recombination sites on both sides of the antibiotic resistance gene, that formed a Neo cassette. The connection between the 5′ end of the Neo cassette and the human IL6 sequence was designed as:

(SEQ ID NO: 12)
5′-AGGCCCGTATTCCAGACCCAAGCTCGTCGACCTGCAGCCAAGCTAT
CGAATTCCTGCAGCCCAATTCCGATCATATTCAATAACCCTTAATATAA
CTTCGTATAATGT-3′,

wherein the last “C” of the sequence “AGCTC” is the last nucleotide of the human sequence, and the first “G” of the sequence “GTCGA” is the first nucleotide of the Neo cassette. The connection between the 3′ end of the Neo cassette with the mouse IL6 locus was designed as

(SEQ ID NO: 13)
5′-CTATACGAAGTTATTAGGTCCCTCGAGGGGATCCACTAGTCTTACC
CAACATGAGCAAGGTCCTAAGTTACATCCAAACA-3′,

wherein the last “T” of the sequence “CTAGT” is the last nucleotide of the Neo cassette, and the first “C” of the sequence “CTTAC” is the first nucleotide of the mouse sequence. In addition, a coding gene with a negative selectable marker (a gene encoding diphtheria toxin A subunit (DTA)) was also inserted downstream of the 3′ homologous arm of the recombinant vector.

The targeting vector was constructed, using restriction enzyme digestion/ligation, and/or artificial gene synthesis, etc. The constructed recombinant vector sequence was initially verified by restriction enzyme digestion, followed by sequencing verification. The correct recombinant vector was electroporated and transfected into embryonic stem cells of C57BL/6 mice. The positive selectable marker gene was used to screen the cells, and the integration of exogenous genes was confirmed by PCR and Southern Blot. Positive clones identified by PCR were further confirmed by Southern Blot (digested with HindIII or SpeI, respectively, and hybridized with 2 probes, see Table 7) to screen out correct positive clone cells.

The following primers were used in PCR:

F1:
(SEQ ID NO: 14)
5′-TGCATCGCATTGTCTGAGTAGG-3′,
R1:
(SEQ ID NO: 15)
5′-ACTTAGGACCTTGCTCATGTTGG-3′;
F2:
(SEQ ID NO: 16)
5′-GCTCGACTAGAGCTTGCGGA-3′,
R2:
(SEQ ID NO: 17)
5′-CAGAAGCCTGATATCTTAGTGTC-3′.

The following probes were used in Southern Blot assays:

Probe 1:
F:
(SEQ ID NO: 18)
5′-CCATGGAAGGAGTTACAGAGA-3′,
R:
(SEQ ID NO: 19)
5′-GTACTGAGGCATATAAAGTTTGC-3′;
Probe 2:
F:
(SEQ ID NO: 20)
5′-GGGACCACTATGGTTGAAT-3′,
R:
(SEQ ID NO: 21)
5′-CAGAAGCCTGATATCTTAGTGTC-3′.

TABLE 7
Length of specific probes and target fragments

| | | | Target size |
|---|---|---|---|
| Restriction Enzyme | Probe | WT size | (correct recombination) |
| HindIII | Probe 1 | 10.1 kb | 13.1 kb |
| SpeI | Probe 2 | 14.2 kb | 6.1 kb |

The positive clones that had been screened (black mice) were introduced into isolated blastocysts (white mice), and the obtained chimeric blastocysts were transferred to the culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then mating the F1 generation heterozygous mice with each other. The positive mice were also mated with the Cre tool mice to remove the positive selectable marker gene (the schematic diagram of the process was shown in FIG. 5), and then the humanized IL6 homozygous mice expressing human IL6 protein can be obtained by mating with each other. The genotype of the progeny mice can be identified by PCR.

In addition, the CRISPR/Cas system can also be used for gene editing. Taking the replacement strategy shown in FIG. 3 as an example, a schematic diagram of the targeting strategy as shown in FIG. 6 was designed. The target sequence determines the targeting specificity of sgRNAs and the efficiency of inducing Cas9 cleavage at the gene of interest. Thus, efficient and specific target sequence selection and design are important for the construction of sgRNA expression vectors. According to the targeting scheme, sgRNA sequences recognizing the 5′ end targeting site (sgRNA1-sgRNA7) and the 3′ end targeting site (sgRNA8-sgRNA15) were designed and synthesized. The 5′ end targeting site and the 3′ end targeting site are located in exon 1 and exon 5 of the mouse IL6 gene, respectively. The targeting site sequences for each sgRNA are shown below:

sgRNA1 target sequence (SEQ ID NO: 22):
5′-AGTCTCAATAGCTCCGCCAGAGG-3′
sgRNA2 target sequence (SEQ ID NO: 23):
5′-GTCTATACCACTTCACAAGTCGG-3′
sgRNA3 target sequence (SEQ ID NO: 24):
5′-GGGCGCCTGCTGCTAGCTGATGG-3′
sgRNA4 target sequence (SEQ ID NO: 25):
5′-TGCTGGCCAACCCACAATGCTGG-3′
sgRNA5 target sequence (SEQ ID NO: 26):
5′-AGTCTCCTGCGTGGAGAAAAGGG-3′
sgRNA6 target sequence (SEQ ID NO: 27):
5′-TGTGCTATCTGCTCACTTGCCGG-3′
sgRNA7 target sequence (SEQ ID NO: 28):
5′-GCCTTCACTTACTTGCAGAGAGG-3′
sgRNA8 target sequence (SEQ ID NO: 29):
5′-ATGCTTAGGCATAACGCACTAGG-3′
sgRNA9 target sequence (SEQ ID NO: 30):
5′-GTCCACAAACTGATATGCTTAGG-3′
sgRNA10 target sequence (SEQ ID NO: 31):
5′-TGCCTAAGCATATCAGTTTGTGG-3′
sgRNA11 target sequence (SEQ ID NO: 32):
5′-AAGTCACTTTGAGATCTACTCGG-3′
sgRNA12 target sequence (SEQ ID NO: 33):
5′-TAAGTCAGATACCTGACAACAGG-3′
sgRNA13 target sequence (SEQ ID NO: 34):
5′-TATTCTGTTACCTAGCCAGATGG-3′
sgRNA14 target sequence (SEQ ID NO: 35):
5′-TTCCAAGAAACCATCTGGCTAGG-3′
sgRNA15 target sequence (SEQ ID NO: 36):
5′-GAACTGACAATATGAATGTTGGG-3′

The UCA kit was used to detect the activities of sgRNAs. The results showed that the guide sgRNAs had different activities (see Table 8 and FIGS. 7A-7B). Accordingly, sgRNA5 and sgRNA13 were preferentially selected for subsequent experiments. Restriction enzyme cleavage sites were added to the 5′ end and the complementary strand to obtain a forward oligonucleotide and a reverse oligonucleotide (see Table 9 for the sequence). After annealing, the annealing products were ligated to the pT7-sgRNA plasmid (the plasmid was first linearized with BbsI), respectively, to obtain expression vectors pT7-sgRNA5 and pT7-sgRNA13.

The pT7-sgRNA vector was synthesized to have a DNA fragment containing the T7 promoter and sgRNA scaffold (SEQ ID NO: 37), and was ligated to the backbone vector (from Takara, Catalog number: 3299) by restriction enzyme digestion (EcoRI and BamHI). The sequences for the plasmids were confirmed by sequencing.

TABLE 8
sgRNA activity test results

| 5′ end targeting site test results | | 3′end targeting site test results | |
|---|---|---|---|
| Con. | 1.00 ± 0.07 | Con. | 1.00 ± 0.08 |
| PC | 203.05 ± 23.16 | PC | 258.00 ± 37.81 |
| sgRNA-1 | 92.04 ± 7.84 | sgRNA-8 | 68.51 ± 8.19 |
| sgRNA-2 | 63.89 ± 51.84 | sgRNA-9 | 22.46 ± 2.28 |
| sgRNA-3 | 57.81 ± 1.77 | sgRNA-10 | 48.06 ± 6.29 |
| sgRNA-4 | 89.46 ± 15.78 | sgRNA-11 | 57.47 ± 12.00 |
| sgRNA-5 | 122.95 ± 58.51 | sgRNA-12 | 65.21 ± 3.08 |
| sgRNA-6 | 68.08 ± 7.85 | sgRNA-13 | 112.72 ± 9.53 |
| sgRNA-7 | 39.02 ± 4.82 | sgRNA-14 | 94.05 ± 8.31 |
| / | / | sgRNA-15 | 37.27 ± 2.80 |

TABLE 9
sgRNA sequences
sgRNA5 sequence

| | |
|---|---|
| SEQ ID NO: 38 | Upstream: 5′-agtctcctgcgtggagaaaa-3′ |
| SEQ ID NO: 39 (forward oligonucleotide) | Upstream: 5′-taggagtctcctgcgtggagaaaa-3′ |
| SEQ ID NO: 40 | Downstream: 5′-ttttctccacgcaggagact-3′ |
| SEQ ID NO: 41 (reverse oligonucleotide) | Downstream: 5′-aaacttttctccacgcaggagact-3′ |

sgRNA13 sequence

| | |
|---|---|
| SEQ ID NO: 42 | Upstream: 5′-tattctgttacctagccaga-3′ |
| SEQ ID NO: 43 (forward oligonucleotide) | Upstream: 5′-taggtattctgttacctagccaga-3′ |
| SEQ ID NO: 44 | Downstream: 5′-tctggctaggtaacagaata-3′ |
| SEQ ID NO: 45 (reverse oligonucleotide) | Downstream: 5′-aaactctggctaggtaacagaata-3′ |

In the schematic diagram of the targeting strategy shown in FIG. 6, wherein the targeting vector comprises a 5′ homologous arm (SEQ ID NO: 46), a 3′ homologous arm (SEQ ID NO: 47), and a DNA fragment comprising the human IL6 sequence (SEQ ID NO: 48). The 5′ homologous arm is identical to nucleotide sequence of 30011619-30013191 of the NCBI accession number NC_000071.6; the 3′ homologous arm is identical to nucleotide sequence of 30019976-30021303 of the NCBI accession number NC_000071.6; and the human IL6 fragment is identical to nucleotide sequence of 22727263-22732018 of the NCBI accession number NC_000007.14. The mRNA sequence of the modified humanized mouse IL6 are shown in SEQ ID NO: 49 (based on transcript NM_000600.4-NP_000591.1) and SEQ ID NO: 50 (based on transcript NM_001318095.1-NP_001305024.1). The corresponding encoded protein sequences are shown in SEQ ID NO: 6 and SEQ ID NO: 8, respectively. The targeting vector can be constructed by routine methods, such as restriction enzyme digestion/ligation and artificial gene synthesis, etc. The constructed recombinant vector can be initially verified by enzymatic digestion, and further confirmed by sequencing. The confirmed plasmids can be used in subsequent experiments.

The pre-mixed Cas9 mRNA, and in vitro transcription products of pT7-sgRNA5 and pT7-sgRNA13 plasmids were injected into the cytoplasm or nucleus the mouse fertilized eggs (B-NDG mice) with a microinjection instrument (using Ambion in vitro transcription kit to carry out the transcription according to the method provided in the product instruction). The embryo microinjection was carried out according to the method described, e.g., in A. Nagy, et al., “Manipulating the Mouse Embryo: A Laboratory Manual (Third Edition),” Cold Spring Harbor Laboratory Press, 2003. The injected fertilized eggs were then transferred to a culture medium for a short time culture, and then was transplanted into the oviduct of the recipient mouse to produce the genetically modified humanized mice (F0 generation).

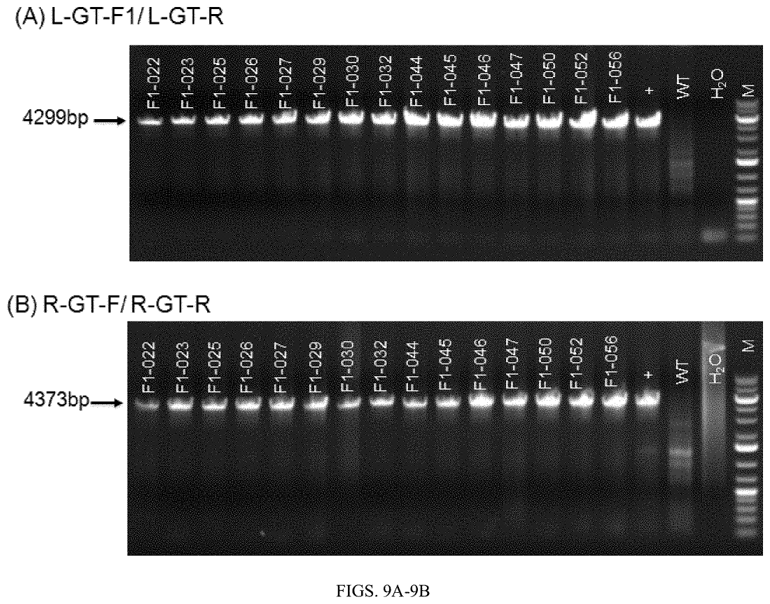

PCR analysis was performed using mouse tail genomic DNA of F0 generation mice. The identification results of some F0 mice are shown in FIGS. 8A-8B. Among them, five mice numbered F0-005, F0-009, F0-021, F0-029 and F0-032 were positive clones. The PCR analysis included the following primers (see Table 10):

TABLE 10
PCR primers and amplified sequence size

| | | Fragment |
|---|---|---|
| Primer | Sequence | Size (bp) |
| L-GT-F1 | 5′-CGGTGAAAGAATGGTGGACTCACTTC-3′ | Mut:4299 |
| | (SEQ ID NO: 51) | |
| L-GT-R | 5′-TGCAGAAGAGAGCCAACCAACCAAA-3′ | |
| | (SEQ ID NO: 52) | |
| R-GT-F | 5′-CCCTGCCCAGCTCATTCTCCACAG-3′ | Mut:4373 |
| | (SEQ ID NO: 53) | |
| R-GT-R | 5′-CCAGAGACTGAGCCACCAATGAGG-3′ | |
| | (SEQ ID NO: 54) | |

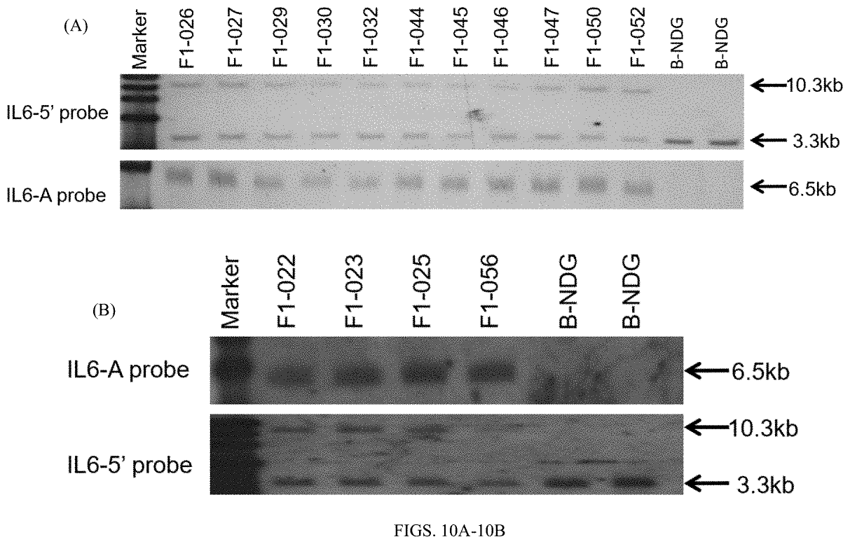

The obtained F0 generation positive clone mice were mated with B-NDG mice to obtain F1 generation mice. The same PCR method can be used to identify the F1 mice, and the results of some F1 mice were shown in FIGS. 9A-9B. The results showed that all 15 F1 generation mice were positive mice. Further detection results using Southern Blot technique are shown in FIGS. 10A-10B. The results showed that all the 15 mice identified as positive by PCR were positive heterozygotes, and no random insertions were detected. This indicated that this method can be used to construct genetically engineered mice without random insertions.

DNA was digested with BglII or ScaI during Southern Blot and hybridized using 2 probes (see Table 11). The probes were as follows:

IL6-5′ Probe:
F:
(SEQ ID NO: 55)
5′-AACAGCTAGCAATGGAGTTGGGCTT-3′,
R:
(SEQ ID NO: 56)
5′-AAAGGTGCTTTTTAAGTCGGGAGCA-3′;
IL6-A Probe:
F:
(SEQ ID NO: 57)
5′-AGGTGAGCTTGGAACTGAACCCAAG-3′,
R:
(SEQ ID NO: 58)
5′-TACCCACTTTTTGTTGCTGCCTGGA-3′.

TABLE 11
Length of specific probes and target fragments

| Restriction enzyme | Probe | WT size | Target size |
|---|---|---|---|
| BgIII | IL6-5′Probe | 3.3 kb | 10.3 kb |
| ScaI | IL6-A Probe | — | 6.5 kb |

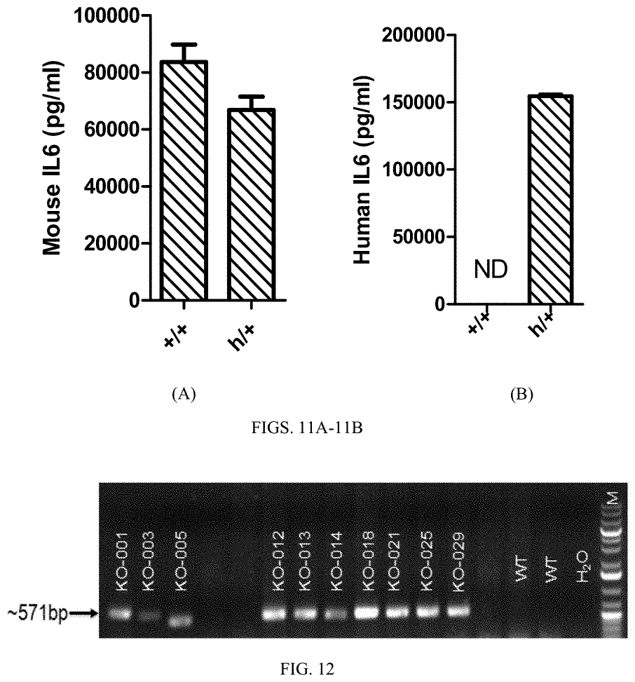

The expression of humanized IL6 protein in mice can be confirmed by routine detection methods. For example, using an ELISA method, one B-NDG mouse (+/+) and one B-NDG background humanized IL6 heterozygote mouse (h/+) were selected and injected intraperitoneally with 20 μg of lipopolysaccharide (LPS). Serum was collected 2 hours later, and mouse or human IL6 protein levels were measured after 1600× or 300× dilution, respectively. As shown in FIGS. 11A-11B, in the stimulated B-NDG mice (+/+), only the expression of mouse IL6 protein was detected, while the expression of human or humanized IL6 protein was not detected. In the stimulated humanized IL6 heterozygous mice (h/+) with B-NDG background, both mouse and human IL6 protein expression were detected.

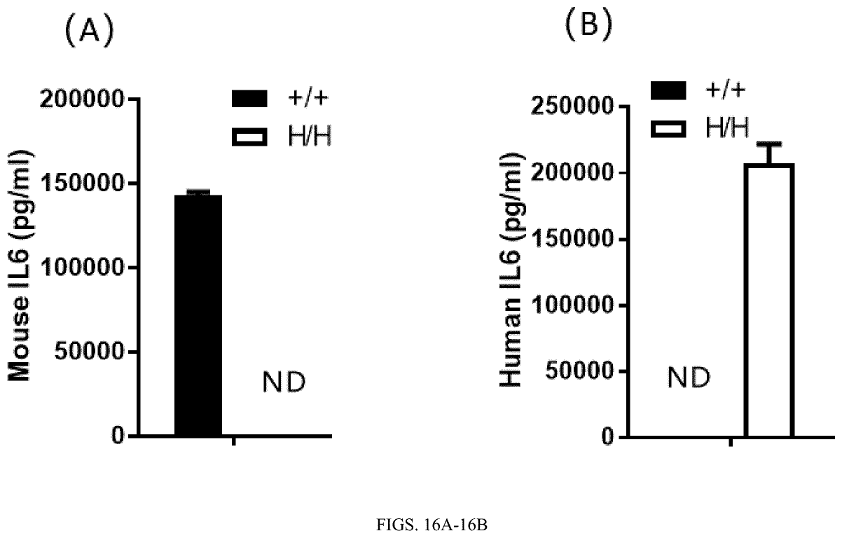

In another experiment, one C57/BL6 mouse (+/+) and one B-NDG background humanized IL6 mouse homozygote (H/H) were selected and the same method was used to detect mouse or human IL6 protein levels. As shown in FIGS. 16A-16B, in LPS-stimulated C57/BL6 mice (+/+), only the expression of mouse IL6 protein was detected, while the expression of human or humanized IL6 protein was not detected. In contrast, only human IL6 protein expression was detected in stimulated humanized IL6 homozygous mouse (H/H), and mouse IL6 protein expression were not detected.

In addition, due to the double-strand break of genomic DNA caused by cleavage of Cas9, the insertion/deletion mutation was randomly generated by repairing through chromosomal homologous recombination. The method herein can also generate gene knockout mice that do not have IL6 protein function, and gene deletion can be detected by routine PCR method. The identification results are shown in FIG. 12. Mice with numbers KO-001, KO-003, KO-005, KO-012, KO-013, KO-014, KO-018, KO-021, KO-025, and KO-029 were IL6 gene knockout mice. PCR analysis was performed using the following primers, and the band obtained from the knockout mice was about 571 bp.

5′MSD-F:
(SEQ ID NO: 68)
5′-ATAAGGTTTCCAATCAGCCCCACCC-3′;
5′MSD-R:
(SEQ ID NO: 69)
5′-ACTTAGGACCTTGCTCATGTTGGGT-3′.

### Example 2: Mice with Humanized IL6R Genes

The mouse IL6R gene (NCBI Gene ID: 16194, Primary source: MGI: 105304, UniProt ID: P08505) is located at 89869324 to 89913196 of chromosome 3 (NC_000069.6), and the human IL6R gene (NCBI Gene ID: 3570, Primary source: HGNC: 6019, UniProt ID: P08887) is located at 154405193 to 154469450 of chromosome 1 (NC_000001.11), both having multiple isoforms or transcripts. FIG. 13 shows the schematic diagram of an exemplary mouse transcript NM_010559.3 (SEQ ID NO: 59) and the corresponding protein sequence NP_034689.2 (SEQ ID NO: 60), and an exemplary human transcript NM_000565.3 (SEQ ID NO: 61) and the corresponding protein sequence NP_000556.1 (SEQ ID NO: 62).

For the purpose of the experiments, a gene sequence encoding the human IL6R protein can be introduced into the endogenous mouse IL6R locus, such that the mouse could express a human IL6R protein. For example, mouse embryonic stem cells can be modified by gene editing techniques. A coding sequence expressing human or humanized IL6R was inserted after the start codon (ATG) sequence in the endogenous mouse IL6R locus. In order to increase the IL6R protein expression level and make the IL6R protein more stable, Woodchuck Hepatitis Virus (WHP) Posttranscriptional Regulatory Element (WPRE) and polyA (polyadenylation) signal sequence were added after the human IL6R coding sequence.

The schematic diagram of the humanized mouse IL6R gene is shown in FIG. 14. The mouse regulated the expression of human IL6R sequence by an endogenous promoter, and the IL6R protein expressed in vivo was human IL6R protein. A targeting strategy was further designed as shown in FIG. 15. The mouse coding region on the humanized mouse IL6R gene would not be transcribed or translated, due to the presence of a stop codon and the polyA signal after the inserted recombinant sequence.

Given that human IL6R or mouse IL6R has multiple isoforms or transcripts, the methods described herein can be applied to other isoforms or transcripts.

As the schematic diagram of the targeting strategy in FIG. 15 shows, the recombinant vector contained homologous arm sequences upstream and downstream of mouse IL6R (about 4.2 kb upstream of the start codon (4133 bp) and about 4.8 kb downstream of the start codon (including the start codon; 4727 bp)), and a DNA fragment (hereinafter named as the fragment of IL-6R-A) comprising the human IL6R sequence and the helper sequences WPRE and polyA (hereinafter named as the fragment of WPRE-PA). Wherein, the upstream homologous arm sequence (5′ homologous arm, SEQ ID NO: 63) is identical to the nucleotide sequence 89917172-89913040 of NCBI accession number NC_000069.6, and the downstream homologous arm sequence (3′ homologous arm, SEQ ID NO: 64) is identical to the nucleotide sequence 89913026-89908300 of NCBI accession number NC_000069.6; within the IL-6R-A fragment sequence (SEQ ID NO: 65), nucleotides 1-1407 encode the human IL6R protein, which is identical to the nucleotide sequence 438-1844 of NCBI accession number NM_000565.3.

The IL-6R-A fragment also included an antibiotic resistance gene for positive clone screening (neomycin phosphotransferase encoding sequence Neo), and two FRT recombination sites on both sides of the antibiotic resistance gene that formed a Neo cassette. The 5′ end of the Neo cassette and the WPRE-PA fragment are connected and the connection was designed as

(SEQ ID NO: 66)
5′-GGGGGAGGATTGGGAAGACAATAGCAGGCATGCTGGGGAGAATTCC
GAAGTTCCTATTCTCTAGAAAGTATAGGAACTTC-3′,

wherein the “A” of the sequence “GCTGGGGA” is the last nucleotide of the WPRE-PA fragment, and the “G” sequence “GAATT” is the first nucleotide of the Neo cassette. The connection between the 3′ end of the Neo cassette and the mouse IL6R locus was designed as 5′-TCTCTAGAAAGTATAGGAACTTCATCAGTCAGGTACATAATGGTGGATCCAGTACTG CTGCACGCTGTTGGTCGCCCTGC-3′ (SEQ ID NO: 67), wherein the last “T” of the sequence “AGTACT” is the last nucleotide of the Neo box, and the first “G” of the sequence “GCTGC” is the first nucleotide of the mouse sequence. In addition, a negative selection marker (a sequence encoding the diphtheria toxin A subunit (DTA)) was also inserted downstream of the 3′ homologous arm of the recombinant vector.

The targeting vector was constructed by restriction enzyme digestion/ligation, and/or sequence synthesis, etc. The constructed recombinant vector sequence can be initially verified by restriction enzyme digestion, followed by sequencing verification. The correct recombinant vector was electroporated and transfected into embryonic stem cells of C57BL/6 mice. The positive selectable marker gene was used to screen the cells, and the integration of exogenous genes was confirmed by PCR and Southern Blot.

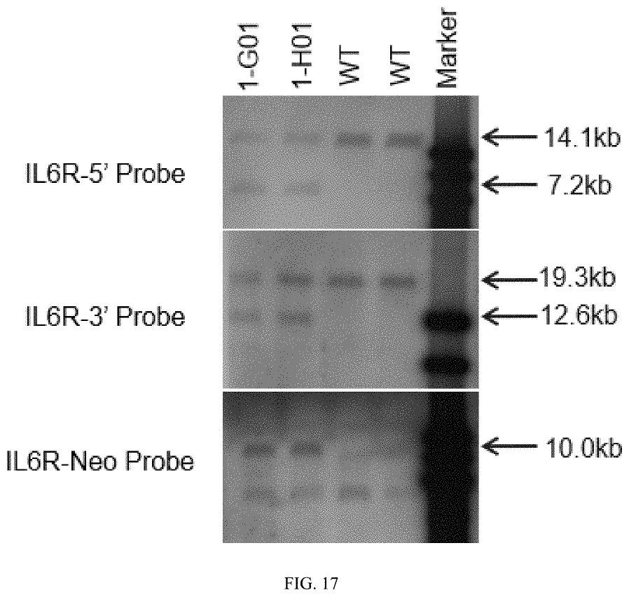

Positive clones identified by PCR can be further confirmed by Southern Blot (digested with SspI, SpeI or EcoRV, respectively, and hybridized with 3 probes, see Table 12) to screen out correct positive clone cells. The test results shown in FIG. 17 indicated that 2 clones (1-G01 and 1-H01) had correct band sizes. Therefore, they were determined as positive heterozygous clones and no random insertions were detected.

TABLE 12
Length of specific probes and target fragments

| Restriction enzyme | Probe | WT size | Targeted size |
|---|---|---|---|
| SspI | IL6R-5′ Probe | 14.1 kb | 7.2 kb |
| SpeI | IL6R-3′ Probe | 19.3 kb | 12.6 kb |
| EcoRV | IL6R-Neo Probe | — | 10.0 kb |

The PCR assay was performed using the following primers:

IL6R-F1:
(SEQ ID NO: 70)
5′-AGCGCACGTCTGCCGCGCTGTTC-3′,
IL6R-R1:
(SEQ ID NO: 71)
5′-TGCCTGTAGGTGACTCTCAAGTCCA-3′;
IL6R-F2:
(SEQ ID NO: 72)
5′-CTGGGATTCCACATCTGTTGTCCAC-3′,
IL6R-R2:
(SEQ ID NO: 73)
5′-ACAGTGGCATTGTCTTCCGGCTCTA-3′.

Southern Blot assays included the following probes:

IL6R-5′ Probe:
F:
(SEQ ID NO: 74)
5′-CTGGGATTCCACATCTGTTGTCCAC-3′,
R:
(SEQ ID NO: 75)
5′-TGCAGCTACCGTTCATGTCCCC-3′;
IL6R-3′ Probe:
F:
(SEQ ID NO: 76)
5′-GTCAACAAGCACAACTCTTCCAGGG-3′,
R:
(SEQ ID NO: 77)
5′-CCAGAGGCTTCTAAACCCTAAAGC-3′;
IL6R-Neo Probe:
F:
(SEQ ID NO: 78)
5′-GGATCGGCCATTGAACAAGAT-3′,
R:
(SEQ ID NO: 79)
5′-CAGAAGAACTCGTCAAGAAGGC-3′.

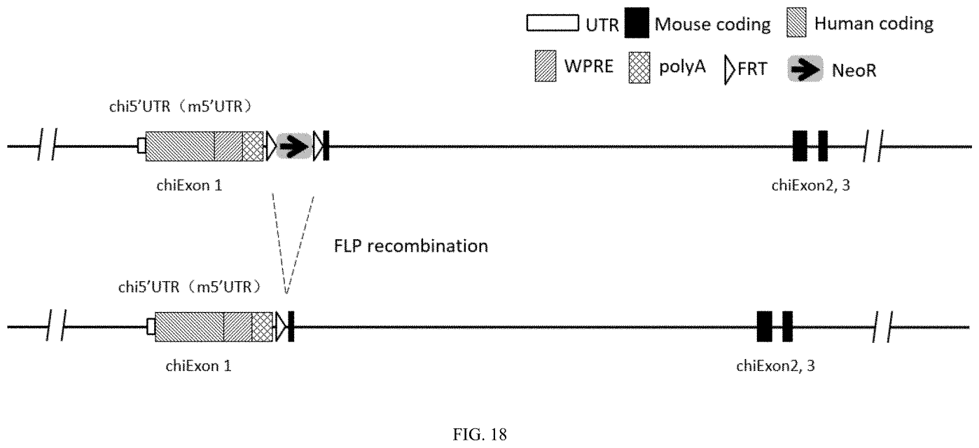

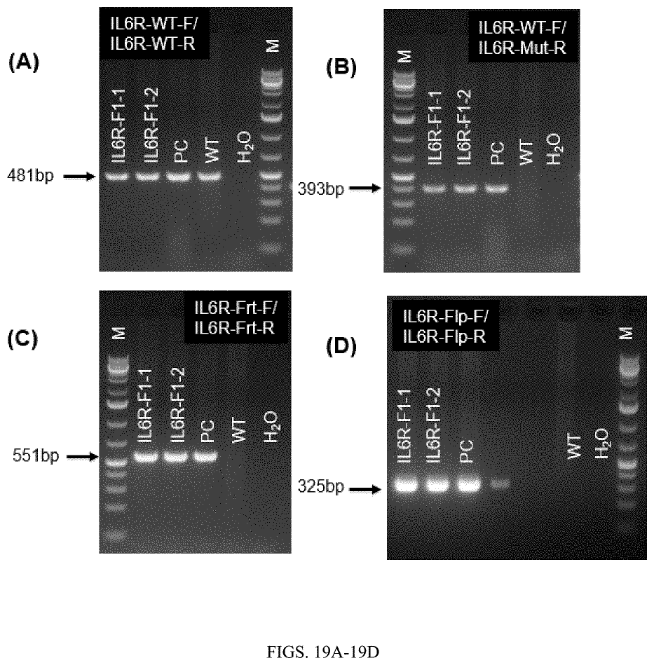

The positive clones that had been screened (black mice) were introduced into isolated blastocysts (white mice), and the obtained chimeric blastocysts were transferred to the culture medium for short-term culture and then transplanted to the fallopian tubes of the recipient mother (white mice) to produce the F0 chimeric mice (black and white). The F2 generation homozygous mice can be obtained by backcrossing the F0 generation chimeric mice with wild-type mice to obtain the F1 generation mice, and then mating the F1 generation heterozygous mice with each other. The positive mice were also mated with the Flp tool mice to remove the positive selectable marker gene (the schematic diagram of the process was shown in FIG. 18), and then the humanized IL6R homozygous mice expressing human IL6R protein can be obtained by mating with each other. The genotype of the progeny mice can be identified by PCR and primers used are shown in Table 13. The results shown in FIGS. 19A-19D indicated that two mice numbered IL6R-F1-1 and IL6R-F-2 were as expected and identified as positive clone mice (wherein PC is a positive control, WT is wild-type). This indicates that the present method can be used to generate genetically engineered IL6R gene humanized mice wherein the humanized gene can be stably passed to the next generation and have no random insertions.

TABLE 13
PCR primers and fragment sizes

| | | Fragment |
|---|---|---|
| Primer | Sequence | size (bp) |
| IL6R-WT-F | 5′-AAATGTTTCACTGTTGCCAGGACGG-3′ | WT:481 |
| | (SEQ ID NO: 80) | |
| IL6R-WT-R | 5′-GACACAGACAGGAGCACGCAGTTAT-3′ | |
| | (SEQ ID NO: 81) | |
| IL6R-WT-F | (SEQ ID NO: 80) | Mut:393 |
| IL6R-Mut-R | 5′-CAGTGGCATTGTCTTCCGGCTCTAC-3′ | |
| | (SEQ ID NO: 82) | |
| IL6R-Frt-F | 5′-GCATCGATACCGTCGACCTCGAC-3′ | Mut:551 |
| | (SEQ ID NO: 83) | |
| L6R-Frt-R | 5′-GACACAGACAGGAGCACGCAGTTAT-3′ | |
| | (SEQ ID NO: 84) | |
| IL6R-F1p-F | 5′-GACAAGCGTTAGTAGGCACATATAC-3′ | Mut:325 |
| | (SEQ ID NO: 85) | |
| IL6R-F1p-R | 5′-GCTCCAATTTCCCACAACATTAGT-3′ | |
| | (SEQ ID NO: 86) | |

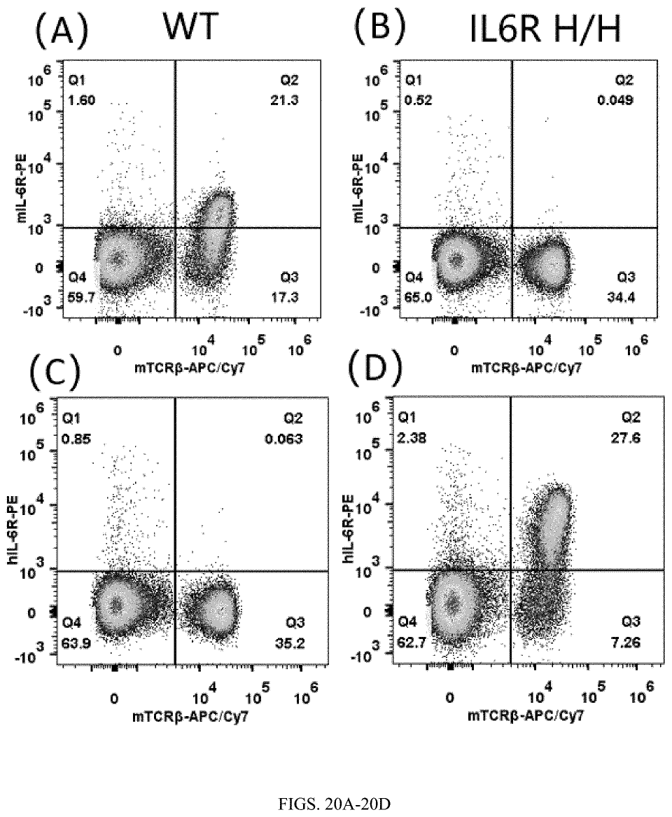

The expression of humanized IL6R protein in mice can be confirmed by routine detection methods. IL6R protein expression was detected by staining the mouse spleen cells with (1) anti-mouse IL6R antibody (mIL-6R PE) combined with murine T cell surface antibody mTcRβ-APC/Cy7, or (2) anti-human IL6R antibody (hTL6R PE) combined with mTcRβ-APC/Cy7, followed by flow cytometry analysis. As shown in FIGS. 20A-20D, flow cytometry results showed that in the spleen of IL6R gene humanized homozygous mice, human IL6R protein can be detected (FIG. 20D), but cells expressing mouse TL6R protein (FIG. 20B) cannot be detected. In the spleen of the wild-type C57BL/6 mice, only the mouse IL6R protein was detected (FIG. 20A), and no cells expressing the human or humanized IL6R protein were detected (FIG. 20C).

### Example 3: Reconstruction of Human Immune System in IL6 Humanized Mice

The humanized IL6 mouse (B-NDG background) prepared by this method can be used as a tool to transplant human CD34+ cells and reconstruct the human immune system. First, mouse bone marrow was cleared by irradiation. Then, cord blood stem cells (CD34+) were intravenously injected to the mouse tail vein. Blood plasma samples were collected and tested at different times after the transplantation. Results showed that IL6 humanized mice have higher percentages of human peripheral blood transplantation than B-NDG controls. In addition, the IL6 humanized mice had a differentiation profile that is more similar to human, and had some mature B cells.

### Example 4: Preparation of Disease Models Using Humanized Mice

Multiple human disease models can be induced/prepared by using the mice described herein. For example, the disease models include multiple sclerosis, asthma, allergy and arthritis. The mice can also be used to test the efficacy of human specific antibodies in vivo. For example, IL6 gene humanized mice can be used to evaluate the pharmacodynamics, pharmacokinetics, and in vivo therapeutic efficacy of IL6 signaling pathway antagonists in various disease models known in the art.

The Experimental Autoimmune Encephalomyelitis (EAE) model was prepared. Humanized IL6 mice (approximately 10 weeks old) prepared by this method were selected and immunized once with Myelin Oligodendrocyte Glycoprotein (MOG) (day 0, subcutaneous injection, 200 μg/mouse). Intraperitoneal injection of Pertussis Toxin (PTX) was given twice (day 0 and day 2, the dose was 400 μg/mouse). Mice were grouped after disease onset, and drugs were administered by gavage or tail vein injection. The in vivo efficacy of different human drugs can be assessed through multiple detection indicators such as behavioral scores, brain/spinal cord IHC pathology, serum/brain homogenate Th17 type multi-cytokine detection, and CNS and spleen flow cytometry.

### Example 5: Evaluation of Drug Efficacy in Humanized Mice

The humanized mice prepared by the method herein can be used to evaluate the drug efficacy of modulators targeting human IL6 or IL6R. For example, in IL6R humanized mice, the homozygous mice were first inoculated with the tumor cell line MC38. When the tumor volume reached about 100 mm³, the mice were divided to a control group and a treatment group based on tumor size. The treatment group was treated with different antibodies against human IL6, and the control group was injected with an equal volume of a blank control. Tumor volume and the mouse weight were periodically measured. The results can be used to effectively evaluate the compound's in vivo safety and efficacy by comparing the changes in mouse weight and tumor size.

### Example 6: Preparation and Identification of Double- or Multi-Humanized Mice

Mice with the humanized IL6 and/or IL6R can also be used to prepare an animal model with double-humanized or multi-humanized genes. As shown in Example 1, in preparing the IL6 gene humanized mice, the fertilized egg cell or embryonic stem cell used in the microinjection and embryo transfer process can be selected from other genetically modified mice, so as to obtain double- or multiple-gene modified mouse models. The fertilized eggs of IL6 humanized mice can also be further genetically engineered to produce mouse lines with one or more humanized or otherwise genetically modified mouse models. In addition, the humanized IL6 and/or IL6R animal model homozygote or heterozygote can be mated with other genetically modified homozygous or heterozygous animal models, and the progeny can be screened. According to Mendel's Law, there is a chance to obtain the double-gene or multiple-gene modified heterozygous animals, and then the heterozygous animals can be mated with each other to finally obtain the double-gene or multiple-gene modified homozygotes. These double- or multi-gene modified mice can be used to verify the in vivo efficacy of human IL6 and/or IL6R-targeting molecules and other gene modulators.

For example, in double-humanized IL6/IL6R mice, because mouse IL6 and IL6R gene are located on chromosome 5 and chromosome 3, respectively, IL6 humanized mice can be mated with IL6R humanized mice to obtain double-humanized IL6/IL6R mice by screening positive clones in progeny mice.

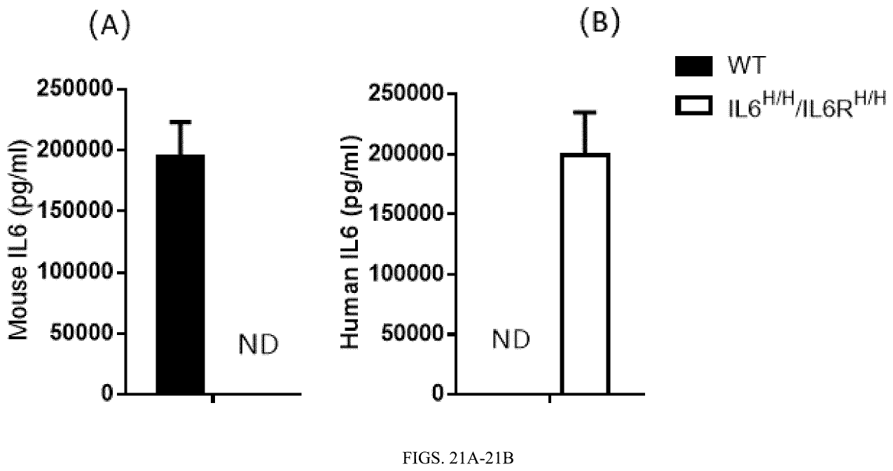

Further, the protein expression can be detected in double-humanized IL6/IL6R mice. One double-humanized IL6/IL6R mouse homozygote (6-7 weeks old, wherein the IL6 humanization strategy is shown in FIG. 2), and one wild-type C57BL/6 mouse (as control) were selected, and the same detection method for IL6 single-gene humanized mice as described above was used to detect mouse and human IL6 protein levels. First, the mice were injected intraperitoneally with 20 μg of LPS. Serum was collected 2 hours later, and mouse or human IL6 protein levels were measured after dilutions. The results (see FIGS. 21A-21B) showed that in LPS-stimulated C57/BL6 mice (WT), only the expression of mouse IL6 protein was detected, while no expression of human or humanized IL6 protein was detected. In contrast, only human IL6 protein expression can be detected in stimulated humanized IL6/IL6R homozygous mouse (IL6^{H/H}/IL6R^{H/H}), and mouse IL6 protein expression cannot be detected.

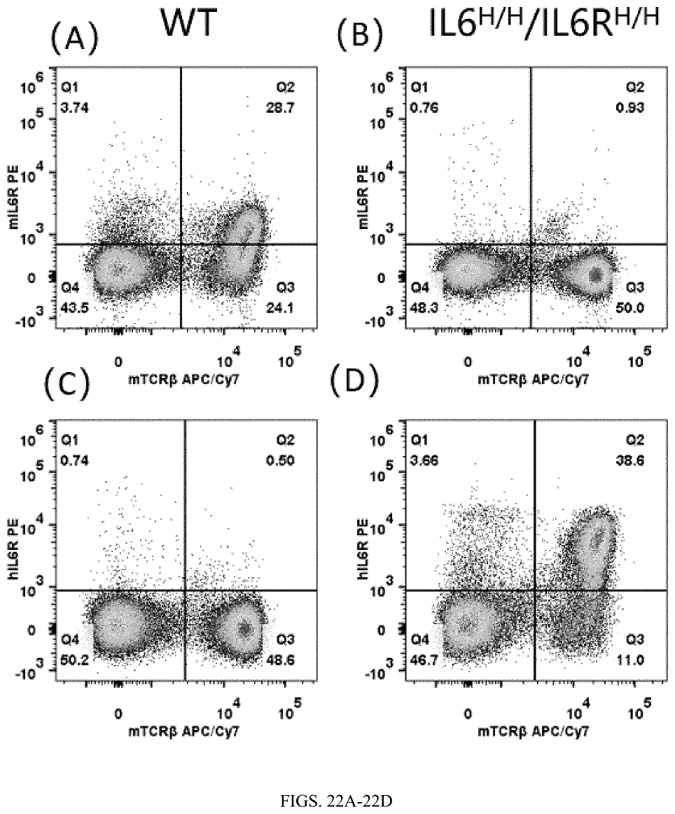

In the next experiment, one double-humanized IL6/IL6R mouse homozygote (same as above), and one wild-type C57BL/6 mouse (as control) were selected, and the same detection method for IL6R single-gene humanized mice as described above was used to detect mouse and human IL6R protein levels by flow cytometry. The results (FIGS. 22A-22D) showed that in the spleen of double-humanized IL6/IL6R mouse homozygote, only the expression of humanized IL6R protein can be detected (FIG. 22D), and cells expressing mouse IL6R protein were not detected (FIG. 22B). In the spleen of wild-type C57BL/6 mice, only mouse IL6R protein can be detected (FIG. 22A), and no cells expressing human or humanized IL6R protein can be detected (FIG. 22C).

### Example 7: Evaluation of Drug Efficacy in Double-Humanized Mice

A collagen-induced arthritis model (CIA) was prepared using the homozygous mice of the double humanized IL6/IL6R mouse prepared by the method herein, and the efficacy of anti-human IL6R antibody was evaluated. The experiment was performed as follows: equal volume of 4 mg/mL chicken type II collagen (Sigma USA) and 4 mg/mL Freund's Complete Adjuvant (Sigma USA) were mixed, and grinded on ice to the water-in-oil state; each double-humanized IL6/IL6R mouse homozygote was injected intradermally at the tail root and multiple points on the back (2-3 points) with 0.1 ml of the above-mentioned mixed solution. On the 21st day after the first immunization, each humanized mouse was subcutaneously injected with 0.1 ml of the above-mentioned mixed solution at multiple locations (2-3 points) again. The control group was injected with equal volume of PBS.

The body weight, toe and arthritis index of each group of mice were monitored twice a week after the second immunization. Paw thickness and the arthritis score of each mouse were recorded for a total of 12 times. Wherein, the arthritis score uses a 4-point scale, 0, normal; 1, redness and swelling of one joint type (A, B, C); 2, redness and swelling of two joint types (A, B, C); 3, redness and swelling of three joint types (A, B, C); 4, the maximum level of redness and swelling of the entire paw. The joint types include: A: interphalangeal joint; B: metacarpophalangeal joint; C: wrist and metatarsal joint. On the 28th day after immunization, mice with a score of at least 1 were grouped (5 mice per group). The specific grouping and dosing schedule are shown in Table 14. Preliminary test results showed that during the experiment, from day 30, the mouse body weight of the CIA model groups G2 and G3 began to decrease, which was significant compared to the control group G1. And the average paw thickness and scoring results showed that anti-human IL6R antibody inhibited the pathogenesis in CIA mouse models, while using hIgG1/kappa did not significantly inhibit the pathogenesis in CIA mouse models.

TABLE 14

| | Modeling | | Dosage/Dosing method/ |
|---|---|---|---|
| Group | reagent | Drug | Frequency |
| G1 | PBS | / | / |
| G2 | CII | h1gG1/kappa | 8 mg/kg/intraperitoneal |
| | | | injection/twice a week for |
| | | | a total of 6 times |
| G3 | CII | Human IL6R | 8 mg/kg/intraperitoneal |
| | | antibody | injection/twice a week for |
| | | | a total of 6 times |

The above research showed that the method herein can be used to evaluate the drug efficacy, pharmacodynamics, pharmacokinetics and in vivo therapeutic efficacy of human specific IL6/IL6R signaling pathway modulators, as well as combined modulators, in autoimmune disease models (Rheumatoid Arthritis) and some other disease models that are known in the art.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-90. (canceled)

91. A genetically-modified, non-human animal whose genome comprises at least one chromosome comprising a sequence encoding a human interleukin-6 receptor (IL6R), wherein the sequence further comprises a Woodchuck Hepatitis Virus (WHP) Posttranscriptional Regulatory Element (WPRE).

92. The animal of claim 91, wherein the sequence encoding the human IL6R is operably linked to an endogenous regulatory element at the endogenous IL6R gene locus in the at least one chromosome.

93. The animal of claim 91, wherein the human IL6R comprises an amino acid sequence that is at least 80% identical to SEQ ID NO: 62.

94. The animal of claim 91, wherein the animal comprises a sequence that is at least 80% identical to SEQ ID NO: 65.

95. The animal of claim 91, wherein the animal is a rodent or a mouse.

96. The animal of claim 91, wherein the animal does not express endogenous IL6R.

97. The animal of claim 91, wherein the animal has one or more cells expressing human IL6R.

98. The animal of claim 91, wherein 3′ end of the sequence is linked to an endogenous sequence starting at the first G of GCTGC in SEQ ID NO: 67.

99. The animal of claim 91, wherein the animal further comprises a sequence encoding human or chimeric IL6.

100. A genetically-modified, non-human animal whose genome comprises at least one chromosome comprising a sequence encoding a human IL6 at an endogenous IL6 gene locus.

101. The animal of claim 100, wherein the sequence encoding the human IL6 is operably linked to an endogenous regulatory element at the endogenous IL6 gene locus in the at least one chromosome.

102. The animal of claim 100, wherein the sequence encoding the human IL6 is operably linked to a human regulatory element at the endogenous IL6 gene locus in the at least one chromosome.

103. The animal of claim 100, wherein the human IL6 comprises an amino acid sequence that is at least 80% identical to SEQ ID NO: 6 or SEQ ID NO: 8.

104. The animal of claim 100, wherein the animal is a rodent or a mouse.

105. The animal of claim 100, wherein the animal has a B-NDG (NOD-Prkd^{scid} L-2rg^{null}) or C57/BL6 background.

106. The animal of claim 100, wherein the animal does not express endogenous IL6.

107. The animal of claim 100, wherein the animal has one or more cells expressing human IL6.

108. The animal of claim 100, wherein the animal further comprises a sequence encoding human or chimeric IL6R.

109. A method of producing a genetically-modified rodent, the method comprising
modifying a genome of an embryo of a rodent by CRISPR associate protein 9 (Cas9) with sgRNAs that target SEQ ID NO: 26 and SEQ ID NO: 34 and a targeting vector, thereby generating a genome in the embryo that comprises at least one chromosome comprising a sequence encoding a chimeric IL6, wherein the targeting vector comprises a 5′ homologous arm, a 3′ homologous arm, and a human IL6 gene fragment; and
transplanting the embryo to a recipient rodent to produce a genetically-modified rodent.

110. The method of claim 109, wherein the rodent is a mouse.
